(19) United States
(12) Patent Application Publication
(10) Pub. No.: US 2024/0175880 A1
(43) Pub. Date: May 30, 2024

(54) LRRC4 FAMILY MIMIC MOLECULES AND DIAGNOSTIC USES THEREOF

(71) Applicant: NEURACLE SCIENCE CO., LTD., Seoul (KR)

(72) Inventors: Jae Young SEONG, Seoul (KR); Hoyun KWAK, Hanam-si (KR); Sitaek OH, Seoul (KR); Min-Hyeok LEE, Seoul (KR)

(73) Assignee: NEURACLE SCIENCE CO., LTD., Seoul (KR)

(21) Appl. No.: 18/552,293

(22) PCT Filed: Nov. 23, 2022
(86) PCT No.: PCT/IB2022/061305

§ 371 Date: Sep. 25, 2023

Related U.S. Application Data
(60) Provisional application No. 63/282,598, filed on Nov. 23, 2021.

(51) Int. Cl. G01N 33/68

(52) U.S. Cl. CPC G01N 33/6896; G01N 2800/28; G01N 2800/52

(57) ABSTRACT

The present disclosure relates to the use of LRRC4 family mimic molecules to determine the expression level of FAM19A5 protein in a subject. In some aspects, such mimic molecules can be used to identify and/or diagnose a subject suffering from or at risk of developing a disease or disorder associated with increased FAM19A5 protein expression. The present disclosure also provides methods of treating subjects identified and/or diagnosed using the methods provided herein.

### CROSS-REFERENCE TO RELATED APPLICATION

This PCT application claims the priority benefit of U.S. Provisional Application No. 63/282,598, filed Nov. 23, 2021, which is herein incorporated by reference in its entirety.

### REFERENCE TO SEQUENCE LISTING SUBMITTED ELECTRONICALLY VIA EFS-WEB

The content of the electronically submitted sequence listing in .XML file (Name: 3763_020PC0_Seqlisting_ST26, Size: 130,483 bytes; and Date of Creation: Nov. 22, 2022) submitted in this application is incorporated herein by reference in its entirety.

### FIELD OF THE DISCLOSURE

The present disclosure provides methods for determining the expression level of a FAM19A5 protein using LRRC4 family mimic molecules. The present disclosure also provides methods of using such LRRC4 family mimic molecules to diagnose a disease or disorder, such as those associated with increased FAM19A5 expression, and/or identify a subject suitable for treatment with a FAM19A5 antagonist.

### BACKGROUND OF THE DISCLOSURE

Diseases and disorders of the central nervous system (CNS) comprise a heterogeneous group of disorders that are typically of unknown etiology and pathogenesis. At present, there exists no cure, let alone methods for identifying a subject responsive to a therapy, for CNS disorders, such as Alzheimer's disease (AD), Parkinson's disease (PD), as well as other related diseases, such as traumatic brain injury (TBI), neuropathic pain, and glaucoma. Indeed, in most instances, available treatments for CNS diseases offer relatively small symptomatic benefit but remain palliative in nature. Similarly, Moreover, with increasing life expectancy and population growth worldwide, the number of individuals afflicted with CNS diseases and disorders is predicted to further increase. Feigin, V. L., et al, Lancet Neurol 16(11): 877-897 (2017). Accordingly, there remains a need for new and more effective approaches to treating and/or diagnosing CNS disorders.

### BRIEF SUMMARY OF THE DISCLOSURE

Provided herein is a method of determining an expression level of a family with sequence similarity 19, member A5 (“FAM19A5”) protein in a subject in need thereof, comprising: (i) contacting a biological sample of the subject with a Leucine Rich Containing 4 (“LRRC4”) family mimic molecule, which is capable of specifically binding to the FAM19A5 protein, and (ii) measuring the expression level of the FAM19A5 protein (“FAM19A5 protein expression level”) in the biological sample.

Also provided herein is a method of diagnosing a disease or disorder in a subject in need thereof, comprising: (i) contacting a biological sample of the subject with a Leucine Rich Containing 4 (“LRRC4”) family mimic molecule, which is capable of specifically binding to a family with sequence similarity 19, member A5 (“FAM19A5”) protein, and (ii) measuring an expression level of the FAM19A5 protein (“FAM19A5 protein expression level”) in the biological sample.

In some aspects, the disease or disorder is associated with an increased FAM19A5 protein expression level. In some aspects, the FAM19A5 protein expression level in the biological sample is increased compared to a reference (e.g., corresponding expression level in a subject who is not afflicted with the disease or disorder). In some aspects, the FAM19A5 protein expression level is increased by about 1-fold, by about 2-fold, by about 3-fold, by about 4-fold, by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, or by about 50-fold compared to the reference.

The present disclosure further provides a method of identifying a subject who is suitable for a treatment with an antagonist against a family with sequence similarity 19, member A5 (“FAM19A5”) protein (“FAM19A5 antagonist”), comprising: (i) contacting a biological sample of the subject with a Leucine Rich Containing 4 (“LRRC4”) family mimic molecule, which is capable of specifically binding to the FAM19A5 protein, and (ii) measuring an expression level of the FAM19A5 protein (“FAM19A5 protein expression level”) in the biological sample, wherein the subject is suitable for the treatment if the FAM19A5 protein expression level is increased compared to a reference (e.g., corresponding expression level in a subject who is not suffering from the disease or disorder). In some aspects, the subject had previously received an administration of the FAM19A5 antagonist.

In some aspects, the FAM19A5 protein expression level is decreased by at least about 5%, by at least about 10%, by at least about 20%, by at least about 30%, by at least about 40%, by at least about 50%, by at least about 60%, by at least about 70%, by at least about 80%, by at least about 90%, or by about 100% compared to the reference.

In some aspects, the method of identifying a subject who is suitable for a treatment with a FAM19A5 antagonist provided herein further comprises administering to the subject an additional dose of the FAM19A5 antagonist after the contacting and measuring.

Also provided herein is a method of treating a disease or disorder associated with an increased expression level of a family with sequence similarity 19, member A5 (“FAM19A5”) protein in a subject in need thereof, comprising administering to the subject a treatment for the disease or disorder and measuring an expression level of the FAM19A5 protein (“FAM19A5 protein expression level”) by contacting a biological sample of the subject with a Leucine Rich Containing 4 (“LRRC4”) family mimic molecule, which is capable of specifically binding to the FAM19A5 protein.

In some aspects, a treatment method provided herein further comprises administering to the subject an additional dose of the treatment if the FAM19A5 protein expression level is increased compared to a reference (e.g., corresponding expression level in a subject who is not afflicted with the disease or disorder). In some aspects, the FAM19A5 protein expression level is increased by about 1-fold, by about 2-fold, by about 3-fold, by about 4-fold, by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, or by about 50-fold compared to the reference.

For any of the methods provided herein, in some aspects, the FAM19A5 protein expression level measured by an enzyme linked immunosorbent assay (ELISA), immunohistochemistry, Western blotting, radioimmunoassay, radical-immunodiffusion, immunoprecipitation assay, Ouchterlony immunodiffusion method, rocket immunoelectrophoresis, tissue immunostaining method, complement fixation assay, FACS, protein chip, SIMOA technology, or any combination thereof. In some aspects, the biological sample comprises a blood, cerebral spinal fluid (CSF), serum, plasma, tissue, cell culture media, saliva, urine, or any combination thereof. In some aspects, the contacting and the measuring is performed in vitro.

For any of the treatment methods provided herein, in some aspects, the treatment comprises an antagonist against the FAM19A5 (“FAM19A5 antagonist), a Leucine Rich Containing 4 (“LRRC4”) family mimic molecule, or both.

In some aspects, the FAM19A5 antagonist comprises an antibody or an antigen-binding portion thereof, antisense oligonucleotide, siRNA, shRNA, miRNA, dsRNA targeting FAM19A5, aptamer, PNA, vector including the same, or any combinations thereof. In some aspects, the FAM19A5 antagonist is an antibody or an antigen-binding portion thereof.

In some aspects, the LRRC4 family mimic molecule of a method provided herein is not an antibody or an antigen-binding portion thereof.

In some aspects, the LRRC4 family mimic molecule comprises a polypeptide comprising, consisting of, or consisting essentially of a domain of a LRRC4 protein family member, wherein the domain is capable of binding to a FAM19A5 protein (“FAM19A5 binding domain”), and wherein the polypeptide is shorter than the corresponding full-length LRRC4 protein family member (SEQ ID NO: 4; SEQ ID NO: 5; or SEQ ID NO: 6).

In some aspects, the FAM19A5 binding domain of a LRRC4 family mimic molecule is about 10 to about 23 amino acids in length. In some aspects, the FAM19A5 binding domain is about 10, about 11, about 12, about 13, about 14, about 15, about 16, about 17, about 18, about 19, about 20, about 21, about 22, or about 23 amino acids in length. In some aspects, the FAM19A5 binding domain is about 10 amino acids in length.

For any of the methods provided herein, in some aspects, the LRRC4 family mimic molecule comprises a polypeptide comprising an amino acid sequence having the following formula (from N-terminus to C-terminus):

A-(T/S)-B, wherein: (Formula I)
(i) A comprises X1-(T/S)-(Y/F)—F-X5; wherein:
   X1 is tyrosine (Y), phenylalanine (F), valine (V), leucine (L), or isoleucine (I);
   (T/S) is threonine (T) or serine (S);
   (Y/F) is tyrosine (Y) or Phenylalanine (F); and
   X5 is any amino acid; and
(ii) B comprises (V/I)-T-V-(E/V); wherein:
   (V/I) is valine (V) or isoleucine (I); and
   (EN) is glutamic acid (E) or valine (V).

For any of the methods provided herein, in some aspects, the LRRC4 family mimic molecule comprises a polypeptide comprising an amino acid sequence having the following formula (from N-terminus to C-terminus):

A-(T/S)-B, wherein: (Formula I)
(i) A comprises (Y/W/M)-(T/Y)-(Y/W)-(F/Y/W)-(T/Y); wherein:
(Y/W/M) is tyrosine (Y), tryptophan (W), or methionine (M);
(T/Y) is threonine (T) or tyrosine (Y);
(Y/W) is tyrosine (Y) or tryptophan (W); and
(F/Y/W) is phenylalanine (F), tyrosine (Y), or tryptophan (W); and
(ii) B comprises X7-(T/S/Y)-X9-X10; wherein:
X7 is valine (V), tyrosine (Y), phenylalanine (F), leucine (L), tryptophan (W), or methionine (M);
(T/S/Y) is threonine (T), serine (S), or tyrosine (Y);
X9 is valine (V), isoleucine (I), tyrosine (Y), phenylalanine (F), leucine (L), tryptophan (W), or methionine (M); and
X10 is glutamic acid (E), aspartic acid (D), isoleucine (I), tyrosine (Y), phenylalanine (F), methionine (M), or tryptophan (W).

For any of the methods provided herein, in some aspects, the LRRC4 family mimic molecule comprises a polypeptide comprising an amino sequence having the following formula (from N-terminus to C-terminus):

X1-X2-X3-F-X5-T-X7-T-V-X10, wherein: (Formula II)
X1 is Y, F, V, L, or I;
X2 is T or S;
X3 is Y or F;
X5 is any amino acid;
X7 is V or I; and/or
X10 is E or V.

For any of the methods provided herein, in some aspects, the LRRC4 family mimic molecule comprises a polypeptide comprising an amino acid sequence having the following formula: (from N-terminus to C-terminus):

X1-X2-X3-X4-X5-X6-X7-X8-X9-X10, wherein: (Formula VI)
X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W.

In some aspects, X1 is Y, F, V, L, or I; X2 is T or S; X3 is Y or F; X4 is F; X5 is T or S; X6 is T; X7 is V or I; X8 is T; X9 is V; X10 is E or V; or combinations thereof.

In some aspects, the LRRC4 family mimic molecule comprises a polypeptide comprising the amino acid sequence set forth in SEQ ID NO: 29 (YTYFTTVTVE). In some aspects, the LRRC4 family mimic molecule comprises a polypeptide consisting of the amino acid sequence set forth in SEQ ID NO: 29 (YTYFTTVTVE). In some aspects, the LRRC4 family mimic molecule comprises a polypeptide comprising the amino acid sequence set forth in SEQ ID NO: 20 (NYSFFTTVTVETTEISPEDTTRK). In some aspects, the LRRC4 family mimic molecule comprises a polypeptide consisting of the amino acid sequence set forth in SEQ ID NO: 20 (NYSFFTTVTVETTEISPEDTTRK). In some aspects, the LRRC4 family mimic molecule comprises a polypeptide comprising the amino acid sequence set forth in SEQ ID NO: 21 (NFSYFSTVTVETMEPSQDERTTR). In some aspects, the LRRC4 family mimic molecule comprises a polypeptide consisting of the amino acid sequence set forth in SEQ ID NO: 21 (NFSYFSTVTVETMEPSQDERTTR). In some aspects, the LRRC4 family mimic molecule comprises a polypeptide comprising the amino acid sequence set forth in SEQ ID NO: 18 (GYTYFTTVTVETLETQPGEE). In some aspects, the LRRC4 family mimic molecule comprises a polypeptide consisting of the amino acid sequence set forth in SEQ ID NO: 18 (GYTYFTTVTVETLETQPGEE).

In some aspects, the amino acid at X2 is phosphorylated or O-glycosylated.

In some aspects, the LRRC4 family mimic molecule comprises a polypeptide, which comprises an amino acid sequence having at least about 60%, at least about 70%, at least about 80%, at least about 90%, at least about 95%, or at least about 99% sequence identity to the amino acid sequence set forth in SEQ ID NO: 29 (YTYFTTVTVE).

In some aspects, the LRRC4 family mimic molecule comprises a polypeptide, which comprises an amino acid sequence that is at least about 70%, at least about 75%, at least about 80%, at least about 85%, at least about 90%, at least about 95%, at least about 96%, at least about 97%, at least about 98%, or about 99% identical to the amino acid sequence set forth in SEQ ID NO: 5, SEQ ID NO: 4, or SEQ ID NO: 6 and contains at least one amino acid modification relative to the amino acid sequence set forth in SEQ ID NO: 5, SEQ ID NO: 4, or SEQ ID NO: 6, respectively. In some aspects, the at least one amino acid modification increases the binding of the polypeptide to the FAM19A5 protein. In some aspects, the at least one amino acid modification increases the stability of the LRRC4 family mimic molecule.

In some aspects, the amino acid residue at position 453 of SEQ ID NO: 5 is T or modified to S or Y; the amino acid residue at position 454 of SEQ ID NO: 5 is T or modified to S or Y; the amino acid residue at position 449 of SEQ ID NO: 5 is Y or modified to F, V, L, I, W, or M; the amino acid residue at position 450 of SEQ ID NO: 5 is T or modified to S or Y; the amino acid residue at position 451 of SEQ ID NO: 5 is Y or modified to F or W; the amino acid residue at position 452 of SEQ ID NO: 5 is F or modified to Y or W; the amino acid residue at position 455 of SEQ ID NO: 5 is V or modified to I, Y, F, L, W, or M; the amino acid residue at position 456 of SEQ ID NO: 5 is T or modified to S or Y; the amino acid residue at position 457 of SEQ ID NO: 5 is V or modified to I, Y, F, L, W, or M; the amino acid residue at position 458 of SEQ ID NO: 5 is E or modified to D, V, I, Y, F, M, or W; or combinations thereof.

In some aspects, the LRRC4 family mimic molecule does not comprise the transmembrane domain and/or the intracellular domain of a member of the LRRC4 protein family.

In some aspects, the LRRC4 family mimic molecule further comprises one or more additional amino acids at the N-terminus, the C-terminus, or both the N-terminus and the C-terminus of LRRC4 family mimic molecule. In some aspects, the one or more additional amino acids are hydrophilic amino acids, D-amino acids, or both.

In some aspects, the N-terminus, C-terminus, or both the N-terminus and the C-terminus of the LRRC4 family mimic molecule comprise a modification which increases the stability of the LRRC4 family mimic molecule. In some aspects, the modification comprises a Fmoc, PEGylation, acetylation, methylation, cyclization, or combinations thereof.

In some aspects, the LRRC4 family mimic molecule is a fusion protein. In some aspects, the LRRC4 family mimic molecule further comprises a half-life extending moiety. In some aspects, the half-life extending moiety comprises a Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, the LRRC4 family mimic molecule comprises a small molecule.

In some aspects, the LRRC4 family mimic molecule is a small molecule of Formula (I):

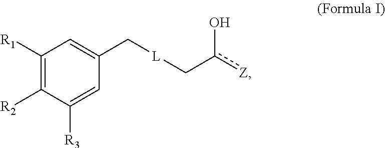

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond.

In some aspects, the LRRC4 family mimic molecule is selected from:

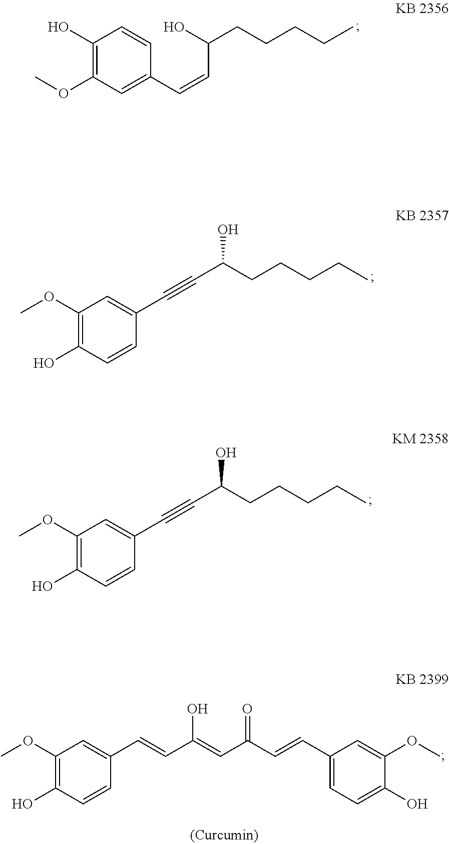

or a pharmaceutically acceptable salt thereof.

In some aspects, the LRRC4 family mimic molecule is a small molecule of Formula (II):

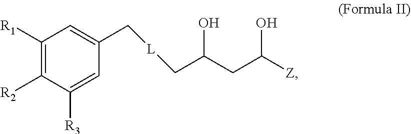

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

In some aspects, the LRRC4 family mimic molecule is selected from:

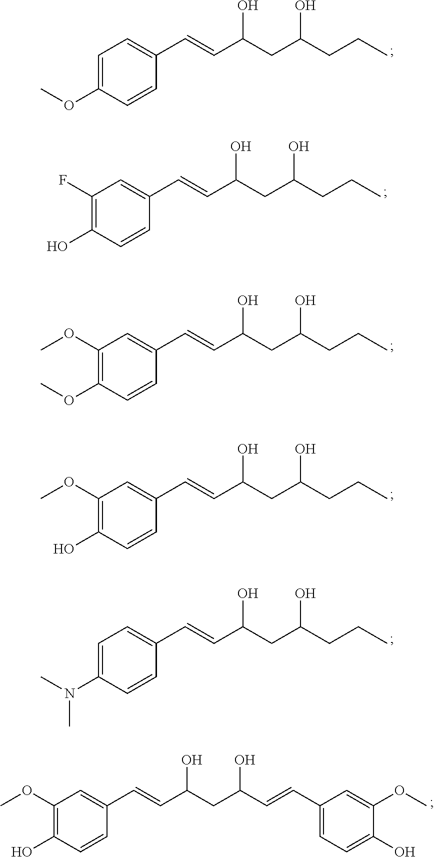

or a pharmaceutically acceptable salt thereof.

In some aspects, the LRRC4 family mimic molecule is a small molecule of Formula (III):

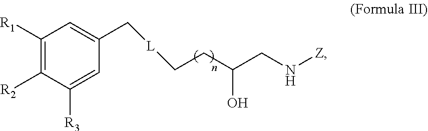

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy,
(iii) L is single, double or triple bond, and
(iv) n is 0 or 1.

In some aspects, the LRRC4 family mimic molecule is selected from:

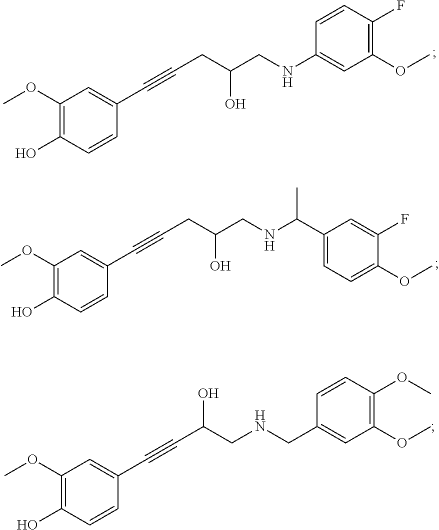

or a pharmaceutically acceptable salt thereof.

In some aspects, the LRRC4 family mimic molecule is capable of competing with members of the LRRC4 protein family for binding to the FAM19A5 protein. In some aspects, the member of the LRRC4 protein family comprises a LRRC4 protein, LRRC4B protein, LRRC4C protein, or combinations thereof.

In some aspects, the disease or disorder comprises an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

### BRIEF DESCRIPTION OF THE DRAWINGS

FIGS. 1A, 1B, and 1C provide schematics of different exemplary approaches of using a sandwich ELISA assay to measure FAM19A5 protein expression in a sample. FIG. 1A shows the use of two different anti-FAM19A5 antibodies (Antibody A conjugated to HRP and Antibody B coated on an ELISA plate) in a sandwich ELISA assay to measure FAM19A5 protein expression in a sample. FIG. 1B shows the use of a LRRC4 family protein (e.g., LRRC4B) conjugated to Fc (“LRRC4B-hFc”) and an anti-FAM19A5 antibody (Antibody A conjugated to HRP) in a sandwich ELISA assay to measure FAM19A5 protein expression in a sample. FIG. 1C shows the use of a LRRC4 family protein (e.g., LRRC4B) conjugated to a 6×His tag, Tobacco Etch Virus (TEV) protease sequence (“His-TEV-LRRC4B”) and an anti-FAM19A5 antibody (Antibody A conjugated to HRP) in a sandwich ELISA assay to measure FAM19A5 protein expression in a sample.

FIG. 2 provides a comparison of FAM19A5 protein level in the blood of mouse (wild-type and FAM19A knock out), rat, monkey (cynomolgus and marmoset), and human, as measured with the LRRC4 family mimic molecule-based sandwich ELISA assay described in FIG. 1.

FIG. 3 shows FAM19A5 protein level in rats after treatment with an anti-FAM19A5 antibody. The rats received a single intravenous administration of the anti-FAM19A5 antibody at one of two doses: 10 mg/kg (“1”) or 50 mg/kg (“2”). Blood was collected from the animals at various time points post-administration, and the FAM19A5 protein level was measured using the LRRC4 family mimic molecule-based sandwich ELISA described in FIG. 1.

FIG. 4 shows FAM19A5 protein and anti-FAM19A5 antibody levels in the blood and CSF, respectively, of rats after anti-FAM19A5 antibody treatment. The rats received a single intravenous administration of the anti-FAM19A5 antibody (10 mg/kg). “Basal”=levels before anti-FAM19A5 antibody administration. FAM19A5 protein level was measured using the LRRC4 family mimic molecule-based sandwich ELISA assay described in FIG. 1.

FIG. 5 shows FAM19A5 protein and anti-FAM19A5 antibody levels in the blood of rats that received weekly intravenous administration of the anti-FAM19A5 antibody. The rats received four total doses (10 mg/kg per dose). Anti-FAM19A5 antibody level was measured using an ELISA method that uses rabbit anti-human IgG Fc and HRP-conjugated goat anti-human IgG kappa light chain antibodies. FAM19A5 protein level was measured using the LRRC4 family mimic molecule-based sandwich ELISA assay described in FIG. 1.

FIG. 6 shows FAM19A5 protein level in the blood of monkey at various time points post anti-FAM19A5 administration. The anti-FAM19A5 antibody was administered at three different doses (3 mg/kg (“1”), 10 mg/kg (“2”), or 30 mg/kg (“3”)), and the monkeys received a single intravenous administration of the antibody. FAM19A5 protein level was measured using the LRRC4 family mimic molecule-based sandwich ELISA assay described in FIG. 1.

FIG. 7 shows FAM19A5 protein level in the blood of traumatic brain injury (TBI)-induced mice treated with anti-FAM19A5 antibody. The mice received the anti-FAM19A5 (“2”) antibody twice (see arrows) (30 mg/kg per dose). Non-treated TBI-induced mice and TBI-induced mice (“1”) that received a human IgG antibody were used as control. FAM19A5 protein level was measured using the LRRC4 family mimic molecule-based sandwich ELISA assay described in FIG. 1. Data are expressed as the mean f SEM.

### DETAILED DESCRIPTION OF THE DISCLOSURE

Disclosed herein is a mimic molecule (e.g., polypeptides and small molecules) that is capable of inhibiting, reducing, and/or dissociating the binding between a FAM19A5 protein and a LRRC4 protein family member (i.e., LRRC4 family mimic molecule). As described herein, the LRRC4 family mimic molecules of the present disclosure resemble members of the LRRC4 protein family and can therefore compete for binding to a FAM19A5 protein. In some aspects, the LRRC4 family mimic molecules described herein exhibit one or more properties (e.g., increased binding affinity and/or stability), such that they can out compete the naturally existing members of the LRRC4 protein family for binding to a FAM19A5 protein. Accordingly, the present application shows for the first time that such LRRC4 family mimic molecules can be used to determine the expression level of a FAM19A5 protein in a subject. Compared to other approaches available in the art, the LRRC4 family mimic molecules of the present disclosure can be used to determine FAM19A5 protein level (e.g., in biofluid, such as plasma, serum, or cerebrospinal fluid) with greater accuracy. Additional aspects of the present disclosure are provided throughout the present application.

To facilitate an understanding of the disclosure disclosed herein, a number of terms and phrases are defined. Additional definitions are set forth throughout the detailed description.

### I. Definitions

Throughout this disclosure, the term “a” or “an” entity refers to one or more of that entity; for example, “a molecule” is understood to represent one or more molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “alkenyl” refers to a group containing hydrogen and carbon and containing at least one carbon-carbon double bond.

As used herein, the term “alkoxy” refers to an alkyl group attached to the parent molecular moiety through an oxygen atom.

As used herein, the term “alkyl” refers to a group containing hydrogen and carbon and containing no double bonds.

As used herein, the term “alkynyl” refers to a group containing hydrogen and carbon and containing at least one carbon-carbon triple bond.

As used herein, the term “amino” refers to —NH₂.

As used herein, the term “bicycloalkenyl” refers to a fused, spirocyclic, or bridged bicyclic hydrocarbon ring system containing at least one double bond.

As used herein, the term “bicycloalkyl” refers to a fused, spirocyclic, or bridged bicyclic cycloalkyl ring.

As used herein, the term “cycloalkenyl” refers to an unsaturated non-aromatic monocyclic hydrocarbon ring system having zero heteroatoms. Representative examples of cycloalkenyl groups include, but are not limited to, cyclopentenyl, cyclohexenyl, cycloheptenyl, and cyclooctenyl.

As used herein, the term “cycloalkyl” refers to a saturated monocyclic hydrocarbon ring system having zero heteroatoms. Representative examples of cycloalkyl groups include, but are not limited to, cyclopropyl, cyclobutyl, cyclopentyl, and cyclohexyl.

As used herein, the term “formyl” refers to —CHO.

As used herein, the terms “halo” and “halogen” refer to Cl, Br, I, or F.

As used herein, the term “haloalkoxy” refers to a haloalkyl group attached to the parent molecular moiety through an oxygen atom.

As used herein, the term “haloalkyl” refers to an alkyl group substituted with one, two, three, or four halogen atoms.

As used herein, the term “heteroaryl” refers to an aromatic ring containing one, two, or three heteroatoms independently selected from nitrogen, oxygen, and sulfur. Representative examples of heteroaryl groups include, but are not limited to, furyl, imidazolyl, pyrazolyl, pyridinyl, pyrrolyl, thiazolyl, and thienyl.

As used herein, the term “heterobicycloalkyl” refers to a non-aromatic bicyclic ring system containing one, two, three, or four heteroatoms independently selected from nitrogen, oxygen, and sulfur and optionally containing one or more double bonds. The heterobicycloalkyl groups of the present disclosure can be attached to the parent molecular moiety through any carbon atom or nitrogen atom in the group.

As used herein, the term “heterocycloalkyl” refers to a non-aromatic ring containing one, two, three, or four heteroatoms independently selected from nitrogen, oxygen, and sulfur and optionally containing one or more double bonds. The heterocycloalkyl groups of the present disclosure can be attached to the parent molecular moiety through any carbon atom or nitrogen atom in the group. Representative examples of heterocycloalkyl groups include, but are not limited to, morpholinyl, piperazinyl, piperidinyl, tetrahydrofuranyl, tetrahydropyranyl, and thiomorpholinyl.

The term “leucine-rich repeat-containing 4 protein family” or “LRRC4 protein family” (including derivatives thereof) refers to a family of proteins that are key synaptic organizers and have been described to play a role in various steps of neural circuit formation, including neuronal migration, neurite outgrowth, and both the formation and functional assembly of synaptic contacts. See, e.g., Woo et al., Mol Cell Neurosci 42(1): 1-10 (September 2009). The LRRC4 protein family includes three members: (1) LRRC4, (2) LRRC4B, and (3) LRRC4C (collectively referred to herein as “LRRC4 protein family member” or “member of the LRRC4 protein family” (or derivatives thereof)). The members of the LRRC4 protein family generally contains nine leucine-rich repeat (LRR) domains flanking LRR N-terminus and C-terminus (see FIG. 3A). These LRR domains are known to interact with fibronectin type III domains of presynaptic receptor protein tyrosine phosphatase (RPTP) proteins. See, e.g., Won et al., Mol Cells 41(7): 622-630 (July 2018). The LRR domains are followed by immunoglobulin-like C2-type (IG) and threonine (Thr)-rich domains, which together form the extracellular portion of members of the LRRC4 protein family. Unlike the other members, the LRRC4B protein has an extra glycine (Gly)-rich domain between the IG and Thr-rich domains. In addition to the extracellular portion, members of the LRRC4 protein family additionally include a transmembrane (TM) domain and a postsynaptic density-binding (PB) domain at the C-terminus of the protein.

In humans, gene encoding the LRRC4 protein is located on chromosome 7 (nucleotides 128,027,071-128,032,107 of GenBank Accession Number NC_000007.14; minus strand orientation). LRRC4 protein synonyms are known and examples include: “Nasopharyngeal Carcinoma-Associated Gene 14 Protein,” “Brain Tumor-Associated Protein BAG,” “Netrin-G2 Ligand,” “NAG14,” “NGL-2,” and “BAG.” The amino acid sequence for the LRRC4 protein is 653 amino acids in length and provided in Table 1 (below). The full-length ectodomain of the LRRC4 protein corresponds to amino acid residues 39-527 of SEQ ID NO: 1 (i.e., SEQ ID NO: 4). Unless indicated otherwise, the term “LRRC4 protein” (including its synonyms) includes any variants or isoforms of the LRRC4 protein which are naturally expressed by cells.

TABLE 1
LRRC4 Protein Sequence

| | |
|---|---|
| Human LRRC4 protein | MKLLWQVTVHHHTWNAILLPFVYLTAQVWILCAAIAAAASAGPQNCPS |
| (UniProt: Q9HBW1) | VCSCSNQFSKVVCTRRGLSEVPQGIPSNTRYLNLMENNIQMIQADTFR |
| (signal peptide is bolded) | HLHHLEVLQLGRNSIRQIEVGAFNGLASLNTLELFDNWLTVIPSGAFE |
| | YLSKLRELWLRNNPIESIPSYAFNRVPSLMRLDLGELKKLEYISEGAF |
| | EGLFNLKYLNLGMCNIKDMPNLTPLVGLEELEMSGNHFPEIRPGSFHG |
| | LSSLKKLWVMNSQVSLIERNAFDGLASLVELNLAHNNLSSLPHDLFTP |
| | LRYLVELHLHHNPWNCDCDILWLAWWLREYIPTNSTCCGRCHAPMHMR |
| | GRYLVEVDQASFQCSAPFIMDAPRDLNISEGRMAELKCRTPPMSSVKW |
| | LLPNGTVLSHASRHPRISVLNDGTLNFSHVLLSDTGVYTCMVTNVAGN |
| | SNASAYLNVSTAELNTSNYSFFTTVTVETTEISPEDTTRKYKPVPTTS |
| | TGYQPAYTTSTTVLIQTTRVPKQVAVPATDTTDKMQTSLDEVMKTTKI |
| | IIGCFVAVTLLAAAMLIVFYKLRKRHQQRSTVTAARTVEIIQVDEDIP |
| | AATSAAATAAPSGVSGEGAVVLPTIHDHINYNTYKPAHGAHWTENSLG |
| | NSLHPTVTTISEPYIIQTHTKDKVQETQI (SEQ ID NO: 1) |
| Human LRRC4 | ASAGPQNCPSVCSCSNQFSKVVCTRRGLSEVPQGIPSNTRYLNLMENN |
| ectodomain protein (i.e., | IQMIQADTFRHLHHLEVLQLGRNSIRQIEVGAFNGLASLNTLELFDNW |
| amino acids 39-527 of | LTVIPSGAFEYLSKLRELWLRNNPIESIPSYAFNRVPSLMRLDLGELK |
| SEQ ID NO: 1) | KLEYISEGAFEGLFNLKYLNLGMCNIKDMPNLTPLVGLEELEMSGNHF |
| | PEIRPGSFHGLSSLKKLWVMNSQVSLIERNAFDGLASLVELNLAHNNL |
| | SSLPHDLFTPLRYLVELHLHHNPWNCDCDILWLAWWLREYIPTNSTCC |
| | GRCHAPMHMRGRYLVEVDQASFQCSAPFIMDAPRDLNISEGRMAELKC |
| | RTPPMSSVKWLLPNGTVLSHASRHPRISVLNDGTLNFSHVLLSDTGVY |
| | TCMVTNVAGNSNASAYLNVSTAELNTSNYSFFTTVTVETTEISPEDTT |
| | RKYKPVPTTSTGYQPAYTTSTTVLIQTTRVPKQVAVPATDTTDKMQTS |
| | LDEVMKTTK (SEQ ID NO: 4) |

In humans, the gene encoding the LRRC4B protein is located on chromosome 19 (nucleotides 50,516,892-50,568,435 of GenBank Accession Number NC_000019.10; minus strand orientation). Synonyms of the LRRC4B protein are known and non-limiting examples include: “Netrin-G3 Ligand,” “LRIG4,” “NGL-3,” “HSM,” and “DKFZp761A179.” The amino acid sequence for the LRRC4B protein is 713 amino acids in length and provided in Table 2 (below). The full-length ectodomain of the LRRC4B protein corresponds to amino acid residues 36-576 of SEQ ID NO: 2 (i.e., SEQ ID NO: 5). Unless indicated otherwise, the term “LRRC4B protein” (including its synonyms) includes any variants or isoforms of the LRRC4B protein which are naturally expressed by cells.

TABLE 2
LRRC4B Protein Sequence

| | |
|---|---|
| Human LRRC4B protein | MARARGSPCPPLPPGRMSWPHGALLFLWLFSPPLGAGGGGVAVTSAAG |
| (UniProt: Q9NT99) | GGSPPATSCPVACSCSNQASRVICTRRDLAEVPASIPVNTRYLNLQEN |
| (signal peptide is bolded) | GIQVIRTDTFKHLRHLEILQLSKNLVRKIEVGAFNGLPSLNTLELFDN |
| | RLTTVPTQAFEYLSKLRELWLRNNPIESIPSYAFNRVPSLRRLDLGEL |
| | KRLEYISEAAFEGLVNLRYLNLGMCNLKDIPNLTALVRLEELELSGNR |
| | LDLIRPGSFQGLTSLRKLWLMHAQVATIERNAFDDLKSLEELNLSHNN |
| | LMSLPHDLFTPLHRLERVHLNHNPWHCNCDVLWLSWWLKETVPSNTTC |
| | CARCHAPAGLKGRYIGELDQSHFTCYAPVIVEPPTDLNVTEGMAAELK |
| | CRTGTSMTSVNWLTPNGTLMTHGSYRVRISVLHDGTLNFTNVTVQDTG |
| | QYTCMVTNSAGNTTASATLNVSAVDPVAAGGTGSGGGGPGGSGGVGGG |
| | SGGYTYFTTVTVETLETQPGEEALQPRGTEKEPPGPTTDGVWGGGRPG |
| | DAAGPASSSTTAPAPRSSRPTEKAFTVPITDVTENALKDLDDVMKTTK |
| | IIIGCFVAITFMAAVMLVAFYKLRKQHQLHKHHGPTRTVEIINVEDEL |
| | PAASAVSVAAAAAVASGGGVGGDSHLALPALERDHLNHHHYVAAAFKA |
| | HYSSNPSGGGCGGKGPPGLNSIHEPLLFKSGSKENVQETQI (SEQ |
| | ID NO: 2) |
| Human LRRC4B | AGGGGVAVTSAAGGGSPPATSCPVACSCSNQASRVICTRRDLAEVPAS |
| ectodomain protein (i.e., | IPVNTRYLNLQENGIQVIRTDTFKHLRHLEILQLSKNLVRKIEVGAFN |
| amino acids 36-576 of | GLPSLNTLELFDNRLTTVPTQAFEYLSKLRELWLRNNPIESIPSYAFN |
| SEQ ID NO: 2) | RVPSLRRLDLGELKRLEYISEAAFEGLVNLRYLNLGMCNLKDIPNLTA |
| | LVRLEELELSGNRLDLIRPGSFQGLTSLRKLWLMHAQVATIERNAFDD |
| | LKSLEELNLSHNNLMSLPHDLFTPLHRLERVHLNHNPWHCNCDVLWLS |
| | WWLKETVPSNTTCCARCHAPAGLKGRYIGELDQSHFTCYAPVIVEPPT |
| | DLNVTEGMAAELKCRTGTSMTSVNWLTPNGTLMTHGSYRVRISVLHDG |
| | TLNFTNVTVQDTGQYTCMVTNSAGNTTASATLNVSAVDPVAAGGTGSG |
| | GGGPGGSGGVGGGSGGYTYFTTVTVETLETQPGEEALQPRGTEKEPPG |
| | PTTDGVWGGGRPGDAAGPASSSTTAPAPRSSRPTEKAFTVPITDVTEN |
| | ALKDLDDVMKTTK (SEQ ID NO: 5) |

In humans, the gene encoding the LRRC4C protein is located on chromosome 11 (nucleotides 40,107,066-41,460,419 of GenBank Accession Number NC_000011.10; minus strand orientation). Synonyms of the LRRC4C protein are known and examples include: “NGL-1,” “Netrin-G1 Ligand,” and “KIAA1580.” The amino acid sequence for the LRRC4C protein is 640 amino acids in length and provided in Table 3 (below). The full-length ectodomain of the LRRC4C protein corresponds to amino acid residues 45-527 of SEQ ID NO: 3 (i.e., SEQ ID NO: 6). Unless indicated otherwise, the term “LRRC4C protein” (including its synonyms) includes any variants or isoforms of the LRRC4C protein which are naturally expressed by cells.

TABLE 3
LRRC4C Protein Sequence

| | |
|---|---|
| Human LRRC4C protein | MLNKMTLHPQQIMIGPRFNRALFDPLLVVLLALQLLVVAGLVRAQTCP |
| (UniProt: Q9HCJ2) | SVCSCSNQFSKVICVRKNLREVPDGISTNTRLLNLHENQIQIIKVNSF |
| (signal peptide is bolded) | KHLRHLEILQLSRNHIRTIEIGAFNGLANLNTLELFDNRLTTIPNGAF |
| | VYLSKLKELWLRNNPIESIPSYAFNRIPSLRRLDLGELKRLSYISEGA |
| | FEGLSNLRYLNLAMCNLREIPNLTPLIKLDELDLSGNHLSAIRPGSFQ |
| | GLMHLQKLWMIQSQIQVIERNAFDNLQSLVEINLAHNNLTLLPHDLFT |
| | PLHHLERIHLHHNPWNCNCDILWLSWWIKDMAPSNTACCARCNTPPNL |
| | KGRYIGELDQNYFTCYAPVIVEPPADLNVTEGMAAELKCRASTSLTSV |
| | SWITPNGTVMTHGAYKVRIAVLSDGTLNFTNVTVQDTGMYTCMVSNSV |
| | GNTTASATLNVTAATTTPFSYFSTVTVETMEPSQDEARTTDNNVGPTP |
| | VVDWETTNVTTSLTPQSTRSTEKTFTIPVTDINSGIPGIDEVMKTTKI |
| | IIGCFVAITLMAAVMLVIFYKMRKQHHRQNHHAPTRTVEIINVDDEIT |
| | GDTPMESHLPMPAIEHEHLNHYNSYKSPFNHTTTVNTINSIHSSVHEP |
| | LLIRMNSKDNVQETQI (SEQ ID NO: 3) |
| Human LRRC4C | QTCPSVCSCSNQFSKVICVRKNLREVPDGISTNTRLLNLHENQIQIIK |
| ectodomain protein (i.e., | VNSFKHLRHLEILQLSRNHIRTIEIGAFNGLANLNTLELFDNRLTTIP |
| amino acids 45-527 of | NGAFVYLSKLKELWLRNNPIESIPSYAFNRIPSLRRLDLGELKRLSYI |
| SEQ ID NO: 3) | SEGAFEGLSNLRYLNLAMCNLREIPNLTPLIKLDELDLSGNHLSAIRP |
| | GSFQGLMHLQKLWMIQSQIQVIERNAFDNLQSLVEINLAHNNLTLLPH |
| | DLFTPLHHLERIHLHHNPWNCNCDILWLSWWIKDMAPSNTACCARCNT |
| | PPNLKGRYIGELDQNYFTCYAPVIVEPPADLNVTEGMAAELKCRASTS |
| | LTSVSWITPNGTVMTHGAYKVRIAVLSDGTLNFTNVTVQDTGMYTCMV |
| | SNSVGNTTASATLNVTAATTTPFSYFSTVTVETMEPSQDEARTTDNNV |
| | GPTPVVDWETTNVTTSLTPQSTRSTEKTFTIPVTDINSGIPGIDEVMK |
| | TTK (SEQ ID NO: 6) |

As used herein, the term “FAM19A5 binding domain” refers to a segment/fragment of a member of the LRRC4 protein family that is capable of binding to a FAM19A5 protein.

The term “family with sequence similarity 19, member A5” or “FAM19A5” refers to a protein that belongs to the TAFA family (also known as FAM19 family) of five highly homologous proteins and is predominantly expressed in brain and the spinal cord. FAM19A5 is also known as “TAFA5” or “Chemokine-like protein TAFA-5.”

In humans, the gene encoding FAM19A5 is located on chromosome 22. There are multiple human FAM19A5 (UniProt: Q7Z5A7) isoforms, which are believed to be produced by alternative splicing: isoform 1 (UniProt: Q7Z5A7-1), which consists of 132 amino acids, isoform 2 (UniProt: Q7Z5A7-2), which consists of 125 amino acids, and isoform 3 (UniProt: Q7Z5A7-3), which consists of 53 amino acids. Human FAM19A5 protein is believed to exist as both membrane bound and soluble (secreted) forms. Isoform 1 is believed to be a membrane protein with one transmembrane region. Isoform 2, which was reported in Tang T. Y. et al., Genomics 83(4):727-34 (2004) as a secreted protein (soluble), contains a signal peptide at amino acid positions 1-25. Isoform 1 is believed to be a membrane protein and predicted based on EST data. Table 4 (below) provides the amino acid sequences of the three known human FAM19A5 isoforms. Unless indicated otherwise, the term “FAM19A5” includes any variants or isoforms of the FAM19A5 protein which are naturally expressed by cells. Accordingly, in some aspects, a polypeptide described herein (e.g., comprising a FAM19A5 binding domain of a member of the LRRC4 protein family) can inhibit the binding of FAM19A5 isoform 1, isoform 2, and/or isoform 3 to the LRRC4 protein family members.

TABLE 4
FAM19A5 Protein Sequences

| | |
|---|---|
| Human FAM19A5 | MAPSPRTGSRQDATALPSMSSTFWAFMILASLLIAYCSQLAAGTCEIV |
| Protein (Isoform 1) | TLDRDSSQPRRTIARQTARCACRKGQIAGTTRARPACVDARIIKTKQW |
| (UniProt: Q7Z5A7-1, | CDMLPCLEGEGCDLLINRSGWTCTQPGGRIKTTTVS (SEQ ID NO: |
| transmembrane protein): | 22) |
| this isoform has been | |
| chosen as the canonical | |
| sequence. | |
| Human FAM19A5 | MQLLKALWALAGAALCCFLVLVIHAQFLKEGQLAAGTCEIVTLDRDSS |
| Protein (Isoform 2) | QPRRTIARQTARCACRKGQIAGTTRARPACVDARIIKTKQWCDMLPCL |
| (UniProt: Q7Z5A7-2, | EGEGCDLLINRSGWTCTQPGGRIKTTTVS (SEQ ID NO: 23) |
| soluble protein) | |
| Human FAM19A5 | MYHHREWPARIIKTKQWCDMLPCLEGEGCDLLINRSGWTCTQPGGRIK |
| Protein (Isoform 3) | TTTVS (SEQ ID NO: 24) |
| (UniProt: Q7Z5A7-3) | |

The term “endogenous,” when used to describe members of the LRRC4 protein family, refers to LRRC4 family proteins that naturally exist in a subject. As described herein, the mimic molecules of the present disclosure differ (structurally and/or functionally) from endogenous LRRC4 protein family members.

“Binding affinity” generally refers to the strength of the sum total of non-covalent interactions between a single binding site of a molecule (e.g., a LRRC4 mimic molecule) and its binding partner (e.g., a FAM19A5 protein). Unless indicated otherwise, as used herein, “binding affinity” refers to intrinsic binding affinity which reflects a 1:1 interaction between members of a binding pair. The affinity of a molecule X (e.g., mimic molecules described herein, which comprise a FAM19A5 binding domain of LRRC4 protein family members) for its partner Y (e.g., FAM19A5) can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE® or kinetic exclusion assay (KinExA).

As used herein, the terms “specifically binds,” “specifically recognizes,” “specific binding,” “selective binding,” and “selectively binds,” are analogous terms and refer to molecules (e.g., LRRC4 family mimic molecules) that bind to an antigen (e.g., FAM19A5 protein) as such binding is understood by one skilled in the art. For example, a molecule that specifically binds to an antigen can bind to other peptides or polypeptides, generally with lower affinity as determined by, e.g., immunoassays, BIACORE®, KinExA 3000 instrument (Sapidyne Instruments, Boise, ID), or other assays known in the art. In some aspects, molecules that specifically bind to an antigen bind to the antigen with a K_{A} that is at least about 2 logs, at least about 2.5 logs, at least about 3 logs, at least about 4 logs or greater than the K_{A} when the molecules bind to another antigen.

As used herein, the term “antigen” refers to any natural or synthetic immunogenic substance, such as a protein, peptide, or hapten. As is apparent from the present disclosure, an antigen can be a FAM19A5 protein or a fragment thereof.

Molecules (e.g., LRRC4 family mimic molecules) that “compete with another protein for binding to a target” refer to molecules that inhibit (partially or completely) the binding of the other protein (e.g., naturally existing members of the LRRC4 protein family) to the target. Whether two compounds compete with each other for binding to a target, i.e., whether and to what extent a LRRC4 family mimic molecule described herein inhibits the binding of the naturally existing members of the LRRC4 protein family to a FAM19A5 protein, can be determined using known competition experiments. In some aspects, a LRRC4 family mimic molecule described herein competes with, and inhibits the binding of the naturally existing members of the LRRC4 protein family to the FAM19A5 protein by at least about 10%, at least about 20%, at least about 30%, at least about 40%, at least about 50%, at least about 60%, at least about 70%, at least about 80%, at least about 90% or at least about 100%. Competition assays can be conducted as described herein or, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999.

Other competitive binding assays that can be used with the present disclosure include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “naturally-occurring” or “naturally-existing,” as used herein, refers to the fact that an object (e.g., protein) can be found in nature. For example, a polypeptide or polynucleotide sequence that is present in an organism (including viruses) that can be isolated from a source in nature and which has not been intentionally modified by man in the laboratory is naturally-occurring. As further described elsewhere in the present disclosure, LRRC4 family mimic molecules useful for the present disclosure are not naturally-occurring.

A “mimic molecule” refers to a molecule that resembles another molecule (“reference molecule”) in structure and/or function. For instance, in some aspects, a mimic molecule can share a partial structure or sequence with the reference molecule, such that mimic molecule can exhibit one or more properties of the reference molecule. However, as is apparent from the present disclosure, a structural or sequence similarity is not always required. In some aspects, a mimic molecule can differ in structure but behave similarly to the reference molecule. As described herein, in some aspects, a mimic molecule comprises a small molecule. In some aspects, a mimic molecule comprises a peptide. In some aspects, a mimic molecule is not an antibody or an antigen-binding portion thereof.

A “polypeptide” refers to a chain comprising at least two consecutively linked amino acid residues, with no upper limit on the length of the chain. One or more amino acid residues in the protein can contain a modification such as, but not limited to, glycosylation, phosphorylation, or disulfide bond formation. A “protein” can comprise one or more polypeptides.

The term “nucleic acids” or “nucleic acid molecule,” as used herein, is intended to include DNA molecules and RNA molecules. A nucleic acid molecule can be single-stranded or double-stranded, and can be cDNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, “administering” refers to the physical introduction of a molecule (e.g., LRRC4 mimic molecule) or a composition comprising the molecule to a subject, using any of the various methods and delivery systems known to those skilled in the art. Non-limiting examples of routes of administration that can be used include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a molecule described herein (e.g., LRRC4 family mimic molecules described herein) can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “subject” includes any human or non-human animal. The term “non-human animal” includes all vertebrates, e.g., mammals and non-mammals, such as non-human primates, sheep, dog, cow, chickens, amphibians, reptiles, etc.

The term “neuron” includes electrically excitable cells that process and transmit information through electrical and chemical signals. Neurons are the major components of the brain and spinal cord of the CNS, and of the ganglia of the peripheral nervous system (PNS), and can connect to each other to form neural networks. A typical neuron is composed of a cell body (soma), dendrites, and an axon. The soma (the cell body) of a neuron contains the nucleus. The dendrites of a neuron are cellular extensions with many branches, where the majority of input to the neuron occurs. The axon is a finer, cable-like projection extending from the soma and carries nerve signals away from the soma and certain types of information back to the soma.

The term “therapeutically effective amount” as used herein refers to an amount of a substance (e.g., LRRC4 mimic molecules described herein), alone or in combination with another therapeutic agent, effective to “treat” a disease or disorder in a subject or reduce the risk, potential, possibility or occurrence of a disease or disorder (e.g., a neurological disease described herein). A “therapeutically effective amount” includes an amount of a substance or a therapeutic agent that provides some improvement or benefit to a subject having or at risk of having a disease or disorder (e.g., a neurological disease described herein). Thus, a “therapeutically effective” amount is an amount that reduces the risk, potential, possibility or occurrence of a disease or provides disorder or some alleviation, mitigation, and/or reduces at least one indicator, and/or decrease in at least one clinical symptom of a disease or disorder.

As used herein, the term “diagnosis” (or derivatives thereof) refers to methods that can be used to determine or predict whether a patient is suffering from a given disease or condition, thereby identifying a subject who is suitable for a treatment. A skilled artisan can make a diagnosis on the basis of one or more diagnostic marker (e.g., FAM19A5), where the presence, absence, amount, or change in amount of the diagnostic marker is indicative of the presence, severity, or absence of the condition. In some aspects, an increase in FAM19A5 protein expression (e.g., in a biological sample from a subject) is indicative of a disease or disorder. Non-limiting examples of such disease or disorder are provided elsewhere in the present disclosure. The term “diagnosis” does not refer to the ability to determine the presence or absence of a particular disease or disorder with 100% accuracy, or even that a given course or outcome is more likely to occur than not. Instead, the skilled artisan will understand that the term “diagnosis” refers to an increased probability that a certain disease or disorder is present in the subject.

### II. Diagnostic Methods

Disclosed herein are methods of determining an expression level of a FAM19A5 protein in a subject in need thereof using a LRRC4 family mimic molecule. As demonstrated herein, in some aspects, a method of determining the expression level of the FAM19A5 protein comprises: (i) contacting a biological sample obtained from the subject with a LRRC4 family mimic molecule (e.g., such as those described herein); and (ii) measuring the expression level of the FAM19A5 protein in the biological sample. In some aspects, a method of determining the expression level of the FAM19A5 protein comprises measuring the expression of level of the FAM19A5 protein in a biological sample obtained from the subject, wherein the biological sample has been contacted with a LRRC4 family mimic molecule.

As is apparent from the present disclosure, the contacting of the biological sample with the LRRC4 family mimic molecule allows the LRRC4 family mimic molecule to specifically bind to a FAM19A5 protein present in the biological sample. As further described herein, in some aspects, a LRRC4 family mimic molecule binds to a FAM19A5 protein with greater binding affinity compared to other binding agents available in the art (e.g., natural or synthetic). Therefore, in some aspects, where the biological sample comprises other FAM19A5 binding agents (e.g., naturally existing members of the LRRC4 protein family), the LRRC4 mimic molecules described herein can out compete other FAM19A5 binding agents for binding to a FAM19A5 protein. In some aspects, when contacted with a biological sample comprising FAM19A5 protein, a LRRC4 family mimic molecule described herein binds to at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the FAM19A5 protein present in the biological sample. In some aspects, the LRRC4 family mimic molecules of the present disclosure bind to all the FAM19A5 protein present in the biological sample.

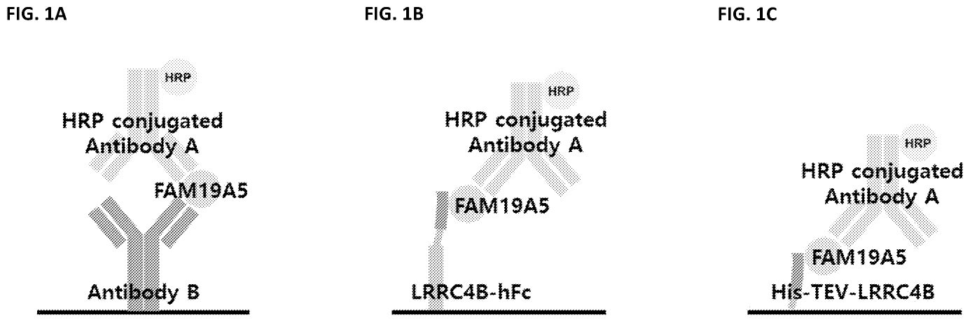

Not to be bound by any one theory, in some aspects, the increased binding affinity of the LRRC4 family mimic molecules to the FAM19A5 protein allows for greater accuracy in determining the FAM19A5 protein level in a biological sample of a subject. For example, many detection assays use antibodies or Fc fragments to capture and measure the level of a protein of interest (i.e., target protein) in a sample (see, e.g., FIGS. 1A and 1B). However, such assays can often have high background, e.g., due to Fc-mediated non-specific binding and/or competition for binding to the target protein with any naturally existing antibodies in the sample. As demonstrated herein, the LRRC4 family mimic molecules of the present disclosure exhibit certain properties (e.g., lacks Fc fragment and binds FAM19A5 protein with high affinity), such that when used to measure FAM19A5 protein level in a sample (see, e.g., FIG. 1C), there is reduced background and the FAM19A5 protein level can be determined more accurately. As used herein, the term “accuracy” (and derivatives thereof) refers to the ability to more closely determine the actual amount (e.g., within 10%) of a material (e.g., FAM19A5 protein) present in a given sample, e.g., as a result of reduced non-specific (i.e., background) signal.

Accordingly, in some aspects, using the methods described herein (e.g., comprising contacting the biological sample with a LRRC4 family mimic molecule), the accuracy of measuring the FAM19A5 protein expression level in a biological sample is increased compared to a reference method (e.g., corresponding method that does not comprise contacting the biological sample with a LRRC4 family mimic molecule described herein, e.g., contacting the biological sample with an anti-FAM19A5 antibody). In some aspects, compared to the reference method, the accuracy is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, or at least about 50-fold.

As described herein, after contacting the biological sample with a LRRC4 family mimic molecule, the methods described above comprises measuring the expression level of the FAM19A5 protein in the biological sample. In some aspects, the measuring step comprises quantifying the amount of LRRC4 family mimic molecule that is bound to a FAM19A5 protein (i.e., “LRRC4 family mimic-FAM19A5 complex”). Any suitable methods/assays known in the art can be used to quantify the amount of LRRC4 family mimic-FAM19A5 complex in the sample. Non-limiting examples include: an enzyme linked immunosorbent assay (ELISA), immunohistochemistry, Western blotting, radioimmunoassay, radical-immunodiffusion, immunoprecipitation assay, Ouchterlony immunodiffusion method, rocket immunoelectrophoresis, tissue immunostaining method, complement fixation assay, FACS, protein chip, Meso Scale Discovery (MSD) ELISA-based assay, SIMOA technology, or any combination thereof. In some aspects, the amount of LRRC4 family mimic-FAM19A5 complex is quantified using the Meso Scale Discovery (MSD) ELISA-based assay. In some aspects, the amount of LRRC4 family mimic-FAM19A5 complex is quantified using SIMOA technology. In some aspects, the amount of LRRC4 family mimic-FAM19A5 complex is quantified using an ELISA assay. In some aspects, the ELISA assay comprises a sandwich ELISA assay, such as that illustrated in FIG. 1C.

As is apparent from the present disclosure, the above-described methods can be useful for a wide range of diagnostic and/or therapeutic purposes. For instance, Applicant has previously demonstrated that certain diseases or disorders can be associated with an abnormal level (e.g., increased) FAM19A5 expression and/or activity. See, e.g., U.S. Pat. No. 11,155,613; US 2020/0157202; US 2020/0300870; US 2021/0070849; WO 2020/079595; US 2020/0223914; US 2020/0299373; and WO 2020/136603, each of which is incorporated herein by reference in its entirety. Accordingly, in some aspects, provided herein is a method of diagnosing a disease or disorder in a subject in need thereof, comprising contacting a biological sample obtained from the subject with a LRRC4 family mimic molecule (e.g., such as those described herein); and (ii) measuring the expression level of the FAM19A5 protein in the biological sample as described herein. In some aspects, a method of diagnosing a disease or disorder comprises measuring the expression of level of the FAM19A5 protein in a biological sample obtained from the subject, wherein the biological sample has been contacted with a LRRC4 family mimic molecule.

In some aspects, an increased expression level of the FAM19A5 protein compared to a reference indicates that the subject is suffering from or at risk of developing the disease or disorder (e.g., associated with increased FAM19A5 expression and/or activity). In some aspects, a reference comprises the corresponding expression level in a biological sample of the subject prior to suffering from or at risk of developing the disease or disorder. In some aspects, a reference comprises the expression level of the FAM19A5 protein in a biological sample obtained from a corresponding subject who is not suffering from or at risk of developing the disease or disorder.

In some aspects, compared to the reference, the expression level of the FAM19A5 protein in the biological sample of the subject is increased by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 35-fold, by at least about 40-fold, by at least about 45-fold, or by at least about 50-fold.

As described herein, the above methods (e.g., directed to determining the expression level of a FAM19A5 protein and/or diagnosing a disease or disorder) comprise contacting a LRRC4 family mimic molecule with a biological sample obtained from the subject. As used herein, the term “biological sample” refers to any sample that contains a material that can be derived from a subject (e.g., human). Non-limiting examples of biological samples useful for the present disclosure include: a blood, cerebrospinal fluid (CSF), interstitial fluid (ISF), serum, plasma, tissue, cell culture media, saliva, urine, or any combination thereof. In some aspects, a biological sample comprises blood. In some aspects, a biological sample comprises CSF. In some aspects, a biological sample comprises serum. In some aspects, a biological sample comprises plasma. In some aspects, a biological sample comprises cell culture media. In some aspects, a biological sample comprises both blood and CSF. In some aspects, a biological sample comprises any combination of blood, CSF, serum, plasma, and culture media. In some aspects, the expression level of the FAM19A5 protein measured in the biological sample correlates with the total expression level of the FAM19A5 protein in the subject.

As further described elsewhere in the present disclosure, Applicant has previously demonstrated that the administration of a FAM19A5 antagonist (e.g., anti-FAM19A5 antibody) can help treat a disease or disorder associated with increased expression level of a FAM19A5 protein.

Accordingly, in some aspects, the present disclosure is directed to a method of identifying a subject who is suitable for treatment with a FAM19A5 antagonist, wherein the method comprises: (i) contacting a biological sample obtained from the subject with a LRRC4 family mimic molecule; and (ii) measuring the expression level of the FAM19A5 protein in the biological sample. As described herein, in some aspects, the measuring step comprises quantifying the amount of LRRC4 family mimic molecule that is bound to a FAM19A5 protein. Any suitable methods/assays (e.g., those described herein) can be used to quantify the amount of LRRC4 family mimic-FAM19A5 complex present in the sample. In some aspects, a method of identifying a subject who is suitable for treating with a FAM19A5 antagonist comprises measuring the expression of level of the FAM19A5 protein in a biological sample obtained from the subject, wherein the biological sample has been contacted with a LRRC4 family mimic molecule.

In some aspects, the subject is suitable for the treatment with a FAM19A5 antagonist if the expression level of the FAM19A5 protein in the biological sample is increased compared to a reference. In some aspects, a reference comprises the corresponding expression level in a biological sample obtained from the subject prior to suffering from or at risk of developing the disease or disorder. In some aspects, a reference comprises the expression level of the FAM19A5 protein in a biological sample obtained from a corresponding subject who is not suffering from or at risk of developing the disease or disorder.

In some aspects, compared to the reference, the expression level of the FAM19A5 protein in the biological sample of the subject who is suitable for treatment with a FAM19A5 antagonist is increased by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 35-fold, by at least about 40-fold, by at least about 45-fold, or by at least about 50-fold.

### II. Therapeutic Methods

In some aspects, the present disclosure is directed to methods of treating a disease or disorder in a subject. For instance, in some aspects, the subject has been identified or diagnosed as suffering from or at risk of developing a disease or disorder, e.g., using the methods described herein (e.g., contacting a biological sample of the subject with a LRRC4 family mimic molecule and measuring the expression level of the FAM19A5 protein in the biological sample). In some aspects, the subject has been identified as being suitable for a treatment, e.g., using the methods described herein.

Accordingly, in some aspects, disclosed herein is a method of treating a disease or disorder associated with an increased expression level of a FAM19A5 protein in a subject in need thereof, comprising measuring an expression level of the FAM19A5 protein in a biological sample obtained from the subject by contacting the biological sample with a LRRC4 family mimic molecule (e.g., those described herein), wherein the subject received an administration of a treatment for the disease or disease prior to the measuring. In some aspects, the measuring step comprises quantifying the amount of LRRC4 family mimic molecule that is bound to a FAM19A5 protein. As described elsewhere in the present disclosure, any suitable methods/assays known in the art and/or described herein can be used to quantify the amount of LRRC4 family mimic-FAM19A5 complex present in the sample.

In some aspects, the expression level of the FAM19A5 protein in the biological sample of the subject is increased compared to a reference. In some aspects, a reference comprises the corresponding expression level in a biological sample of the subject prior to suffering from or at risk of developing the disease or disorder. In some aspects, a reference comprises the expression level of the FAM19A5 protein in a biological sample obtained from a corresponding subject who is not suffering from or at risk of developing the disease or disorder. In some aspects, compared to the reference, the expression level of the FAM19A5 protein in the biological sample of the subject is increased by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 35-fold, by at least about 40-fold, by at least about 45-fold, or by at least about 50-fold.

In some aspects, where the expression level of the FAM19A5 protein is increased compared to the reference, the method of treating a disease or disorder provided herein further comprises administering to the subject an additional dose of the treatment. In some aspects, the additional dose is the same as the dose previously administered to the subject. In some aspects, additional dose is greater than the dose previously administered to the subject. In some aspects, the additional dose is less than the dose previously administered to the subject. In some aspects, the additional dose is administered to the subject at the same dosing interval as previously administered to the subject. In some aspects, the additional dose is administered to the subject at a different dosing interval (e.g., longer or shorter). In some aspects, the additional dose can be administered in combination with an additional therapeutic agent. Non-limiting examples of such additional therapeutic agents are provided elsewhere in the present disclosure.

In some aspects, where the expression level of the FAM19A5 protein is increased compared to the reference, the method comprises stopping the treatment. Where the treatment is stopped, in some aspects, the method comprises administering a different treatment to the subject (e.g., a different FAM19A5 antagonist).

In some aspects, the level of the FAM19A5 protein is the same compared to the reference (e.g., expression level of the FAM19A5 protein in a biological sample obtained from a corresponding subject who is not suffering from or at risk of developing the disease or disorder). In some aspects, the expression level of the FAM19A5 protein is the same to that of the reference, where the expression level of the FAM19A5 protein is at least about 80%, at least about 85%, at least about 90%, at least about 95%, or about 100% of the corresponding expression level of the reference. Accordingly, when used to compare the amount of FAM19A5 protein in the sample to that of the reference, the term “same” comprises both an identical amount (i.e., 100% identity) and an amount that is very close (e.g., within about 20%) to the amount present in the reference.

In some aspects, where the expression level of the FAM19A5 protein is similar compared to the reference, the method of treating a disease or disorder provided herein further comprises administering to the subject an additional dose of the treatment. In some aspects, the additional dose is the same as the dose previously administered to the subject. In some aspects, additional dose is greater than the dose previously administered to the subject. In some aspects, the additional dose is less than the dose previously administered to the subject. In some aspects, the additional dose is administered to the subject at the same dosing interval as previously administered to the subject. In some aspects, the additional dose is administered to the subject at a different dosing interval (e.g., longer or shorter). In some aspects, the additional dose can be administered in combination with an additional therapeutic agent (e.g., those described herein).

In some aspects, where the expression level of the FAM19A5 protein is similar compared to the reference, the method comprises stopping the treatment. Where the treatment is stopped, in some aspects, the method comprises administering a different treatment to the subject (e.g., a different FAM19A5 antagonist).

In some aspects, the expression level of the FAM19A protein is decreased compared to the reference (e.g., expression level of the FAM19A5 protein in a biological sample obtained from a corresponding subject who is not suffering from or at risk of developing the disease or disorder). In some aspects, compared to the reference, the expression level of the FAM19A5 protein in the biological sample of the subject is decreased by at least about 5%, by at least about 10%, by at least about 15%, by at least about 20%, by at least about 25%, by at least about 30%, by at least about 35%, by at least about 40%, by at least about 45%, by at least about 50%, by at least about 55%, by at least about 60%, by at least about 65%, by at least about 70%, by at least about 75%, by at least about 80%, by at least about 85%, by at least about 90%, by at least about 95%, or by about 100%.

In some aspects, where the expression level of the FAM19A5 protein is decreased compared to the reference, the treatment is maintained (e.g., the subject continues to receive the same treatment, e.g., at the same dose and/or dosing interval). In some aspects, where the expression level of the FAM19A5 protein is decreased, the treatment is altered. For instance, in some aspects, a treatment is altered where the subject receives a lower dose of the treatment. In some aspects, a treatment is altered where the subject receives the same treatment (e.g., at the same dose) but at a longer dosing interval. In some aspects, a treatment is altered where the subject receives a lower dose of the treatment at a longer dosing interval.

As is apparent from at least the above disclosure, in some aspects, the above disclosure can be useful in determining whether a subject is responding to a treatment for a disease or disorder described herein (e.g., associated with increased FAM19A5 protein expression). Accordingly, in some aspects, the present disclosure is directed to a method of identifying a subject who is responsive to a treatment for a disease or disorder associated with an increased expression level of a FAM19A5 protein, comprising measuring an expression level of the FAM19A5 protein in a biological sample obtained from the subject by contacting the biological sample with a LRRC4 family mimic molecule (e.g., those described herein), wherein the subject received an administration of the treatment prior to the measuring. In some aspects, the measuring step comprises quantifying the amount of LRRC4 family mimic molecule that is bound to a FAM19A5 protein. As described elsewhere in the present disclosure, any suitable methods/assays known in the art and/or described herein can be used to quantify the amount of LRRC4 family mimic-FAM19A5 complex present in the sample.

In some aspects, the subject is responsive to the treatment if the expression level of the FAM19A5 protein is decreased compared to the reference (e.g., expression level of the FAM19A5 protein in a biological sample obtained from a corresponding subject who is not suffering from or at risk of developing the disease or disorder). In some aspects, the expression level of the FAM19A5 protein is decreased by at least about 5%, by at least about 10%, by at least about 20%, by at least about 30%, by at least about 40%, by at least about 50%, by at least about 60%, by at least about 70%, by at least about 80%, by at least about 90%, or by about 100% compared to the reference. In some aspects, where the subject is identified as being responsive to the treatment, the treatment is maintained (e.g., same dose and/or dosing interval), altered (e.g., lower dose and/or longer dosing interval), or stopped as described herein.

In some aspects, the subject is not responsive to the treatment if the expression level of the FAM19A5 protein is increased, same, or similar compared to the reference (e.g., expression level of the FAM19A5 protein in a biological sample obtained from a corresponding subject who is not suffering from or at risk of developing the disease or disorder). In some aspects, where the subject is identified as not being responsive to the treatment, the treatment is stopped and a different treatment is administered to the subject. In some aspects, where the subject is not responsive to the treatment, the subject is treated with a higher dose of the treatment. In some aspects, where the subject is not responsive to the treatment, the subject is treated with the same dose but at a shorter dosing interval. In some aspects, where the subject is not responsive to the treatment, the subject is treated with a higher dose of the treatment and at a shorter dosing interval. In some aspects, where the subject is not responsive to the treatment, the treatment can be administered to the subject in combination with an additional therapeutic agent (e.g., those described herein).

In any of the methods provided herein comprising administering to the subject a treatment (e.g., for a disease or disorder associated with increased expression level of a FAM19A5 protein), in some aspects, the treatment comprises a FAM19A5 antagonist. In some aspects, the treatment comprises a LRRC4 family mimic molecule. In some aspects, the treatment comprises both a FAM19A5 antagonist and a LRRC4 family mimic molecule.

In some aspects, a FAM19A5 antagonist useful for the present disclosure comprises an antisense oligonucleotide, a siRNA, a shRNA, a miRNA, a dsRNA, an aptamer, a PNA, a vector including the same, or any combinations thereof. In some aspects, a FAM19A5 antagonist comprises an antibody, or an antigen-binding portion thereof, that specifically binds to the FAM19A5 protein (collectively referred to herein as “anti-FAM19A5 antibody”), a polynucleotide encoding the anti-FAM19A5 antibody, or a vector comprising the polynucleotide thereof. Non-limiting examples of such FAM19A5 antagonists are described, e.g., in US 2019/0300599; US 2020/0299373; US 2021/0054062; and WO 2020/141452; each of which is incorporated herein by reference in its entirety. Additional disclosure relating to LRRC4 family mimic molecules that are useful for the present disclosure are provided elsewhere in the present disclosure.

In some aspects, when administered to a subject, a FAM19A5 antagonist useful for the present disclosure can inhibit, slow down, suppress, curb, reduce, reverse, and/or prevent gliosis and its associated detrimental effects to the CNS of a subject. In some aspects, when administered to a subject, a FAM19A5 antagonist can inhibit, slow down, suppress, curb, reduce, reverse, and/or prevent excessive or abnormal proliferation of reactive astrocytes and its associated detrimental effects to the CNS of the subject. In some aspects, when administered to a subject, a FAM19A5 antagonist can stimulate, promote, increase, and/or activate the growth of neurons in the subject. In some aspects, when administered to a subject, a FAM19A5 antagonist can enhance and/or promote the survival of neurons in the subject. In some aspects, when administered to a subject, a FAM19A5 antagonist can enhance and/or promote the regrowth of an axon in the subject. In some aspects, when administered to a subject, a FAM19A5 antagonist can increase a neurite outgrowth and/or synapse formation in neurons in the subject.

In some aspects, when administered to a subject, a LRRC4 family mimic molecule described herein can inhibit, reduce, and/or dissociate the interaction between a FAM19A5 protein and members of the LRRC4 protein family. In some aspects, after the administration, the formation of the complex between the FAM19A5 protein and members of the LRRC4 family mimic molecule is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, compared to a reference formation (e.g., corresponding value in the subject prior to the administration or the value in a corresponding subject who did not receive the administration).

As described elsewhere in the present disclosure, the binding of a FAM19A5 protein to members of the LRRC4 protein family can inhibit the activity of the LRRC4 protein family members. For instance, in some aspects, the formation of the FAM19A5-LRRC4 family protein complexes can lead to impaired neural circuit formation, resulting in an imbalance in the dynamic gain and loss of synapses, which is fundamental to the healthy function of neurons in the central and peripheral nervous systems.

Accordingly, in some aspects, the decrease in the formation of the complex between a FAM19A5 protein and members of the LRRC4 protein family can increase the activity of the members of the LRRC4 protein family. In some aspects, after the administration, the activity of the members of the LRRC4 protein family is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding value in the subject prior to the administration or value in a corresponding subject that did not receive the administration). Non-limiting examples of such activity can include neurite outgrowth, neuronal migration, and the formation and functional assembly of synaptic contacts.

Not to be bound by any one theory, in some aspects, the one or more therapeutic effects of a FAM19A5 antagonist and/or LRRC4 family mimic molecule described above can help treat or reduce one or more symptoms of a disease or disorder described herein (e.g., associated with increased FAM19A5 expression). Non-limiting examples of such diseases or disorders include an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.

In some aspects, provided herein is a method of treating an ALS in a subject in need thereof, comprising administering to the subject a treatment for ALS and measuring an expression level of a FAM19A5 protein by contacting a biological sample of the subject with a LLRC4 family mimic molecule described herein. In some aspects, the treatment for ALS comprises a FAM19A5 antagonist, a LRRC4 family mimic molecule, or both. In some aspects, ALS that can be treated with present disclosure comprises a sporadic ALS, familial ALS, or both. As used herein, the term “sporadic” ALS refers to ALS that is not associated with any family history of ALS occurrence. Approximately about 90% or more of the ALS diagnosis are for sporadic ALS. As used herein, the term “familial” ALS refers to ALS that occurs more than once within a family, suggesting a genetic component to the disease. In some aspects, ALS that can be treated with the present disclosure comprises primary lateral sclerosis (PLS). PLS can affect upper motor neurons in the arms and legs. More than 75% of people with apparent PLS, however, develop lower motor neuron signs within four years of symptom onset, meaning that a definite diagnosis of PLS cannot be made until then. PLS has a better prognosis than classic ALS, as it progresses slower, results in less functional decline, does not affect the ability to breathe, and causes less severe weight loss. In some aspects, ALS comprises progressive muscular atrophy (PMA). PMA can affect lower motor neurons in the arms and legs. While PMA is associated with. longer survival on average than classic ALS, it still progresses to other spinal cord regions over time, eventually leading to respiratory failure and death. Upper motor neuron signs can develop late in the course of PMA, in which case the diagnosis might be changed to classic ALS.

In some aspects, provided herein is a method of treating an Alzheimer's disease in a subject in need thereof, comprising administering to the subject a treatment for the Alzheimer's disease and measuring an expression level of a FAM19A5 protein by contacting a biological sample of the subject with a LLRC4 family mimic molecule described herein. In some aspects, the treatment for the Alzheimer's disease comprises a FAM19A5 antagonist, a LRRC4 family mimic molecule, or both. Not to be bound by any one theory, in some aspects, treating an Alzheimer's disease comprises reducing an amyloid beta (AB) plaque load in the subject (e.g., suffering from Alzheimer's disease). As used herein, “amyloid beta plaque” refers to all forms of aberrant deposition of amyloid beta including large aggregates and small associations of a few amyloid beta peptides and can contain any variation of the amyloid beta peptides. Amyloid beta (AP) plaque is known to cause neuronal changes, e.g., aberrations in synapse composition, synapse shape, synapse density, loss of synaptic conductivity, changes in dendrite diameter, changes in dendrite length, changes in spine density, changes in spine area, changes in spine length, or changes in spine head diameter. In some aspects, an increase in AP plaque load can result in synapse loss in neurons.

In some aspects, provided herein is a method of treating a glaucoma in a subject in need thereof, comprising administering to the subject a treatment for the glaucoma and measuring an expression level of a FAM19A5 protein by contacting a biological sample of the subject with a LLRC4 family mimic molecule described herein. In some aspects, the treatment for the glaucoma comprises a FAM19A5 antagonist, a LRRC4 family mimic molecule, or both.

In some aspects, provided herein is a method of treating a retinopathy in a subject in need thereof, comprising administering to the subject a treatment for the retinopathy and measuring an expression level of a FAM19A5 protein by contacting a biological sample of the subject with a LLRC4 family mimic molecule described herein. In some aspects, the treatment for retinopathy comprises a FAM19A5 antagonist, a LRRC4 family mimic molecule, or both. In some aspects, a retinopathy that can be treated with the present disclosure comprises a diabetic retinopathy. The term “diabetic retinopathy” comprises all types of diabetic retinopathy including, but not limited to, non-proliferative diabetic retinopathy (NPDR), proliferative diabetic retinopathy (PDR), diabetic maculopathy, and diabetic macular edema. Not to be bound by any one theory, in some aspects, treating a retinopathy (e.g., diabetic retinopathy) comprises improving a retinal potential in a subject in need thereof. In some aspects, an improved retinal potential comprises an increase in the values for A-wave, B-wave, and/or oscillatory potential, compared to a reference (e.g., corresponding subject that was not treated with a LRRC4 family mimic molecule described herein). It will be apparent to those skilled in the arts that treating retinopathies can be useful in treating other types of eye disorders, including but not limited to, macular degeneration and glaucoma.

In some aspects, present disclosure provides a method of treating a neuropathic pain in a subject in need thereof, comprising administering to the subject a treatment for the neuropathic pain and measuring an expression level of a FAM19A5 protein by contacting a biological sample of the subject with a LLRC4 family mimic molecule described herein. In some aspects, the treatment for the neuropathic pain comprises a FAM19A5 antagonist, a LRRC4 family mimic molecule, or both. In some aspects, a neuropathic pain is a central neuropathic pain, i.e., a pain due to injury or damage affecting any level of the CNS (e.g., brain injury and spinal cord injury), including the central somatosensory nervous system, or associated with or as a result of a disease or disorder such as stoke, multiple sclerosis, or lateral medullary infarction. In some aspects, a neuropathic pain is a peripheral neuropathic pain, a pain due to injury or damage affecting any level of the peripheral nerves system (e.g., injury of a motor nerve, a sensory nerve, an autonomic nerve, or a combination thereof), or resulting from or associated with a disease or disorder

In some aspects, provided herein is a method of treating a spinal cord injury in a subject in need thereof, comprising administering to the subject a treatment for the spinal cord injury and measuring an expression level of a FAM19A5 protein by contacting a biological sample of the subject with a LLRC4 family mimic molecule described herein. In some aspects, the treatment for the spinal cord injury comprises a FAM19A5 antagonist, a LRRC4 family mimic molecule, or both. In some aspects, provided herein is a method of treating a traumatic brain injury in a subject in need thereof, comprising administering to the subject a treatment for the traumatic brain injury and measuring an expression level of a FAM19A5 protein by contacting a biological sample of the subject with a LLRC4 family mimic molecule described herein. In some aspects, the treatment for the traumatic brain injury comprises a FAM19A5 antagonist, a LRRC4 family mimic molecule, or both. In some aspects, provided herein is a method of treating a stroke in a subject in need thereof, comprising administering to the subject a treatment for the stroke and measuring an expression level of a FAM19A5 protein by contacting a biological sample of the subject with a LLRC4 family mimic molecule described herein. In some aspects, the treatment for the stroke comprises a FAM19A5 antagonist, a LRRC4 family mimic molecule, or both.

In some aspects, provided herein is a method of treating a Parkinson's disease in a subject in need thereof, comprising administering to the subject a treatment for the Parkinson's disease and measuring an expression level of a FAM19A5 protein by contacting a biological sample of the subject with a LLRC4 family mimic molecule described herein. In some aspects, the treatment for the Parkinson's disease comprises a FAM19A5 antagonist, a LRRC4 family mimic molecule, or both. As used herein, the term “Parkinson's disease” (PD) refers to neurodegenerative disorder leading to motor and non-motor manifestations (i.e., symptoms) and characterized by extensive degeneration of dopaminergic neurons in the nigrostriatal system. Non-limiting examples of motor and non-motor manifestations of PD are provided elsewhere in the present disclosure. Proteinopathy (α-synuclein abnormal aggregation) is a hallmark of PD. Other exemplary features of PD include dopaminergic neuron damage, mitochondrial dysfunction, neuroinflammation, protein homeostasis (e.g., autophagic clearance of damaged proteins and organelles glial cell dysfunction), and combinations thereof.

Additionally, any of the treatment described herein (e.g., FAM19A5 antagonist and/or LRRC4 family mimic molecule) can be administered to a subject in combination with one or more additional therapeutic agents. Any therapeutic agents useful for treating a disease or disorder associated with increased FAM19A5 protein expression can be used in combination with the treatments described herein. In some aspects, the additional therapeutic agent comprises an acetylcholinesterase inhibitor. In some aspects, the additional therapeutic agent comprises a dopamine agonist. In some aspects, the additional therapeutic agent comprises a dopamine receptor antagonist. In some aspects, the additional therapeutic agent comprises an antipsychotic. In some aspects, the additional therapeutic agent comprises a monoamine oxidase (MAO) inhibitor. In some aspects, the additional therapeutic agent comprises a catechol O-methyltransferase (COMT) inhibitor. In some aspects, the additional therapeutic agent comprises a N-methyl-D-aspartate (NMDA) receptor antagonist. In some aspects, the additional therapeutic agent comprises an immunomodulatory. In some aspects, the additional therapeutic agent comprises an immunosuppressant.

Non-limiting examples of such agents include: Tetrabenazine (XENAZINE®), antipsychotic drugs, such as haloperidol (HALDOL®), chlorpromazine, risperidone (RIDPERDAL®), quetiapine (SEROQUEL®), levodopa (with or without Carbidopa) (LODOSYN®), dopamine agonists such as pramipexole (MIRAPEX®), ropinirole (REQUIP®), and rotigotine (NEUPRO®), and apomorphine (APOKYN®), selegiline (ELDEPRYL®, ZELAPAR®), rasagiline (AZILECT®), Entacapone (COMTAN®), benztropine (COGENTIN®), trihexyphenidyl, amantadine, Donepezil (ARICEPT®), Galantamine (RAZADYNE®), Rivastigmine (EXELON®), Glatiramer acetate (COPAXONE®), Dimethyl fumarate (TECFIDERA®), Fingolimod (GILENYA®), Teriflunomide (AUBAGIO®), Natalizumab (TYSABRI®), Alemtuzumab (LEMTRADA®), Mitoxantrone (NOVANTRONE®), riluzole (RILUTEK®), physostigmine salicylate (ANTILIRIUM®), physostigmine sulfate (ESERINE®), metrifonate, neostigmine, ganstigmine, pyridostigmine (MESTINON®), ambenonium (MYTELASE®), demarcarium, Debio 9902 (also known as ZT-1®; Debiopharmladostigil, NP-0361), tacrine (COGNEX®), tolserine, velnacrine maleate, memoquin, huperzine A (HUP-A®; NeuroHitech), phenserine, edrophonium (ENLON®, TENSILON®), INM-176, apomorphine (APOKYN®), bromocriptine (PARLODEL®), cabergoline (DOSTINEX®), dihydrexidine, dihydroergocryptine, fenoldopam (CORLOPAM®), lisuride (DOPERGIN®), terguride spergolide (PERMAX®), piribedil (TRIVASTAL®, TRASTAL®), quinpirole, SKF-82958 (GlaxoSmithKline), cariprazine, pardoprunox, sarizotan, chlorpromazine, fluphenazine loxzpine, resperidone, thioridazine, thiothixene, trifluoperazine, 7-hydroxyamoxapine, droperidol (INAPSINE®, DRIDOL®, DROPLETAN®), domperidone (MOTILIUM®), L-741742, L-745870, raclopride, SB-277011A, SCH-23390, ecopipam, SKF-83566, metoclopramide (REGLAN®), lurasidone (LATUDA®, also known as SM-13496; Dainippon Sumitomo), aripiprazole (ABILIFY®), chlorpromazine (THORAZINE®), iloperidone (FANAPTA®), flupentixol decanoate (DEPIXOL®, FLUANXOL®), reserpine (SERPLAN®), pimozide (ORAP®), fluphenazine decanoate, fluphenazine hydrochloride, prochlorperazine (COMPRO®), asenapine (SAPHRIS®), loxapine (LOXITANE®), molindone (MOBAN®), perphenazine, thioridazine, thiothixine, trifluoperazine (STELAZINE®), ramelteon, clozapine (CLOZARIL®), norclozapine (ACP-104), paliperidone (INVEGA®), melperone, olanzapine (ZYPREXA®), talnetant, amisulpride, ziprasidone (GEODON®), blonanserin (LONASEN®), ACP-103 (Acadia Pharmaceuticals), selegiline hydrochloride (I-deprenyl, ELDEPRYL®, ZELAPAR®), dimethylselegilene, brofaromine, phenelzine (NARDIL®), tranylcypromine (PARNATE®), moclobemide (AURORIX®, MANERIX®), befloxatone, safinamide, isocarboxazid (MARPLAN®), nialamide (NIAMID®), iproniazide (MARSILID®, IPROZID®, IPRONID®), CHF-3381 (Chiesi Farmaceutici), iproclozide, toloxatone (HUMORYL®, PERENUM®), bifemelane, desoxypeganine, harmine (also known as telepathine or banasterine), harmaline, linezolid (ZYVOX®, ZYVOXID®), pargyline (EUDATIN®, SUPIRDYL®), nitecapone, tolcapone (TASMAR®), tropolone, memantine (NAMENDA®, AXURA®, EBIXA®), amantadine (SYMMETREL®), acamprosate (CAMPRAL®), besonprodil, ketamine (KETALAR®), delucemine, dexanabinol, dexefaroxan, dextromethorphan, dextrorphan, traxoprodil, CP-283097, himantane, idantadol, ipenoxazone, L-701252 (Merck), lancicemine, levorphanol (DROMORAN®), LY-233536 and LY-235959 (both Lilly), methadone, (DOLOPHINE®), neramexane, perzinfotel, phencyclidine, tianeptine (STABLON®), dizocilpine (also known as MK-801), EAB-318 (Wyeth), ibogaine, voacangine, tiletamine, aptiganel (CERESOTAT®), gavestinel, remacimide, MBP-8298 (synthetic myelin basic protein peptide), roquinimex (LINOMIDE®), laquinimod (also known as ABR-215062 and SAIK-MS), ABT-874 (human anti-IL-12 antibody; Abbott), rituximab (RITUXAN®), leflunomide, ciclesonide, daclizumab (ZENAPAX®), methotrexate (TREXALL®, RHEUMATREX®), suplatast tosilate, mycophenolate mofetil (CELLCEPT®), mycophenolate sodium (MYFORTIC®), azathioprine (AZASAN®, IMURAN®), mercaptopurine (PURI-NETHOL®), cyclophosphamide (NEOSAR®, CYTOXAN®), voclosporin, PUR-118, AMG 357, AMG 811, BCT197, chlorambucil (LEUKERAN®), cladribine (LEUSTATIN®, MYLINAX®), alpha-fetoprotein, etanercept (ENBREL®), leflunomide, ciclesonide chloroquine, hydroxychloroquine, d-penicillamine, auranofin, sulfasalazine, sodium aurothiomalate, cyclosporine, cromolyn, infliximab, adalimumab, certolizumab pegol, golimumab, rituximab, ocrelizumab, ofatumumab, 4-benzyloxy-5-((5-undecyl-2H-pyrrol-2-ylidene)methyl)-2,2′-bi-1H-pyrrole (also known as PNU-156804), and combinations thereof.

In some aspects, any of the treatments described herein (e.g., a FAM19A5 antagonist and/or LRRC4 family mimic molecule) can be administered to the subject intravenously, orally, parenterally, transthecally, intrathecally, intra-cerebroventricularly, intravitreally, pulmonarily, subcutaneously, intradermally, intramuscularly, or intraventricularly.

### II. Mimic Molecules

Disclosed herein are mimic molecules of members of the LRRC4 protein family (referred to herein as “LRRC4 family mimic molecules”) that can be used with the methods of the present disclosure. As is apparent from the present disclosure, while the LRRC4 family mimic molecules described herein share certain properties with the members of the LRRC4 protein family, the LRRC4 family mimic molecules differ (structurally and/or functionally) from naturally existing members of the LRRC4 protein family. For instance, in some aspects, a LRRC4 family mimic molecule useful for the present disclosure comprises a small molecule. As further described elsewhere in the present disclosure, in some aspects, a LRRC4 family mimic molecule comprises a polypeptide, wherein the polypeptide comprises, consists of, or consists essentially of a domain of a member of the LRRC4 protein family, wherein the domain is capable of binding to the FAM19A5 protein (also referred to herein as the “FAM19A5 binding domain”). For such aspects, the polypeptides can comprise one or more amino acid substitutions within the FAM19A5 binding domain. As described elsewhere in the present disclosure, in some aspects, such amino acid substitutions can improve one or more properties of the polypeptides, e.g., increase the stability and/or binding affinity of the polypeptides to the FAM19A5 protein. In some aspects, the polypeptides can comprise the FAM19A5 binding domain but lack one or more other domains of the members of the LRRC4 protein family. For instance, in some aspects, a polypeptide comprises the FAM19A5 binding domain, but does not comprise the transmembrane domain. In some aspects, a polypeptide comprises the FAM19A5 binding domain, but does not comprise the intracellular domain of members of the LRRC4 protein family (e.g., postsynaptic density-binding (PB) domain). In some aspects, a polypeptide comprises the FAM19A5 binding domain, but does not comprise both the transmembrane domain and the intracellular domain. Accordingly, in some aspects, polypeptides described herein are shorter than the naturally existing LRRC4 protein family members. Additionally, in carrying out their biological activity (e.g., neural circuit formation), members of the LRRC4 protein family (LRRC4, LRRC4B, and LRRC4C) interact with their ligand (netrin-G2, receptor tyrosine phosphatase LAR, and netrin-G1, respectively). See, e.g., Li et al., Mol Cancer 13: 266 (December 2014). Because the polypeptides of the present disclosure do not comprise all the domains of LRRC4 protein family members, in some aspects, the polypeptides do not bind to the LRRC4 protein family ligands and instead, specifically target the FAM19A5 protein. Accordingly, in some aspects, the polypeptides described herein do not replace the endogenous LRRC4 protein family members. Instead, in some aspects, by inhibiting, reducing, and/or dissociating the interaction between FAM19A5 and members of the LRRC4 protein family, the polypeptides of the present disclosure can free up the endogenous LRRC4 family proteins and allow them to carry out their natural biological activity.

Where the LRRC4 family mimic molecules comprise small molecule compounds, in some aspects, the mimic molecule is:

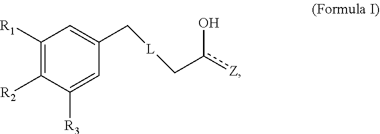

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond.

In some aspects, R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy. In some aspects, R₁, R₂ and R₃ are selected from hydrogen, hydroxy, methoxy, ethoxy, n-propyloxy, iso-propyloxy, n-butoxy. In some aspects, R₁, R₂ and R₃ are selected from hydrogen, hydroxy, and methoxy. In some aspects, R₁ and R₂ are selected from hydroxy and methoxy and R₃ is hydrogen.

In some aspects, Z is selected from a straight chain or branched (C₁-C₈)alkyl, a straight chain or branched (C₂-C₈)alkenyl, a straight chain or branched (C₂-C₈)alkynyl, and —CH—C(O)—CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy. In some aspects, Z is selected from a straight chain or branched (C₁-C₈)alkyl, a straight chain or branched (C₂-C₈)alkenyl, and —CH—C(O)—CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxyl.

In some aspects, the LRRC4 family mimic molecule is a small molecule of formula (I) wherein:
(i) R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, and —CH—C(O)—CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy; and
(iv) L is a double or triple bond.

In some aspects, the LRRC4 family mimic molecule is a small molecule of formula (I) wherein:
(i) R₁, R₂ and R₃ are selected from hydrogen, hydroxy, methoxy, ethoxy, n-propyloxy, iso-propyloxy, and n-butoxy;
(ii) [symbol] is a single or double bond;
(iii) Z is selected from a straight chain or branched (C₁-C₈)alkyl, a straight chain or branched (C₂-C₈)alkenyl, and —CH—C(O)—CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxy; and
(iv) L is a double or triple bond.

In some aspects, the LRRC4 family mimic molecule is selected from:

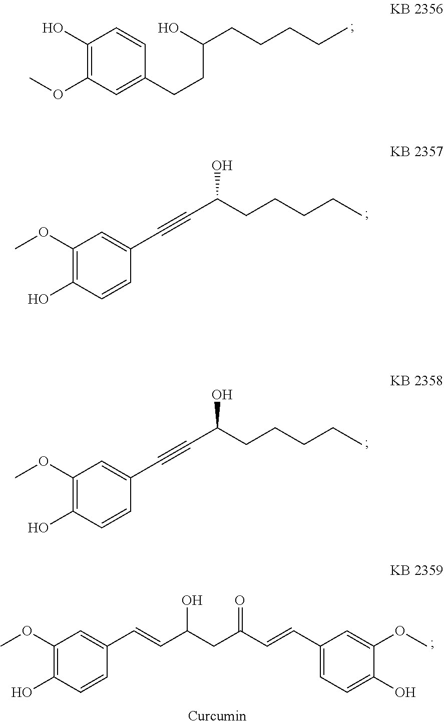

or a pharmaceutically acceptable salt thereof.

In some aspects, the LRRC4 family mimic molecule is:

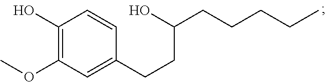 [SMILES: CCCCCC(O)CCc1ccc(O)c(OC)c1]

or a pharmaceutically acceptable salt thereof.

In some aspects, the LRRC4 family mimic molecule is:

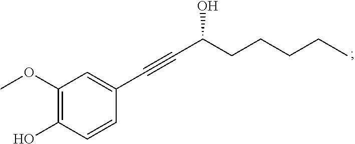 [SMILES: CCCCC[C@@H](O)C#Cc1ccc(O)c(OC)c1]

or a pharmaceutically acceptable salt thereof.

In some aspects, the LRRC4 family mimic molecule is:

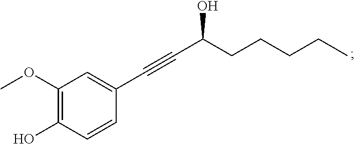 [SMILES: CCCCC[C@H](O)C#Cc1ccc(O)c(OC)c1]

or a pharmaceutically acceptable salt thereof.

In some aspects, the LRRC4 family mimic molecule is:

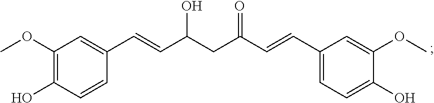

or a pharmaceutically acceptable salt thereof.

In some aspects, the LRRC4 family mimic molecule is:

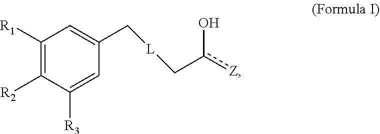

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond, and wherein the LRRC4 family mimic molecule is not selected from:

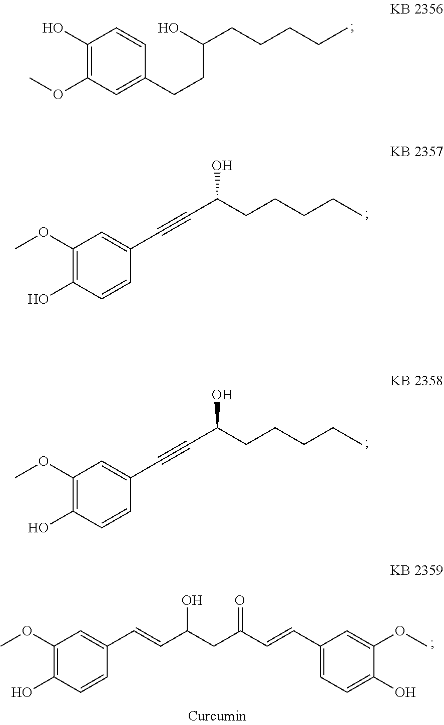

or a pharmaceutically acceptable salt thereof.

In some aspects, the LRRC4 family mimic molecule is:

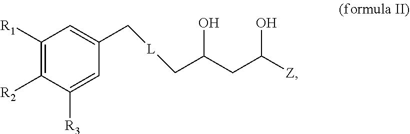

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

In some aspects, R₁, R₂ and R₃ are selected from hydrogen, fluoro, chloro, bromo, hydroxy, methoxy, ethoxy, n-propyloxy, iso-propyloxy, n-butoxy, trifluoromethoxy, difluoromethoxy, fluoromethoxy, amino, N-methylamino, N-ethylamino, N—N-propylamino, and N,N-dimethylamino. In some aspects, R₁, R₂ and R₃ are selected from hydrogen, fluoro, chloro, bromo, hydroxy, methoxy, ethoxy, n-propyloxy, amino, N-methylamino, N-ethylamino, N—N-propylamino, and N,N-dimethylamino. In some aspects, R₁, R₂ and R₃ are selected from hydrogen, fluoro, hydroxy, methoxy, and N,N-dimethylamino. In some aspects, R₁ and R₂ are selected from fluoro, hydroxy, methoxy, and N,N-dimethylamino, and R3 is hydrogen.

In some aspects, Z is selected from a straight chain or branched (C₁-C₈)alkyl, a straight chain or branched (C₂-C₈)alkenyl, a straight chain or branched (C₂-C₈)alkynyl, and —CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy. In some aspects, Z is selected from a straight chain or branched (C₁-C₈)alkyl, a straight chain or branched (C₂-C₈)alkenyl, and —CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxyl.

In some aspects, the LRRC4 family mimic molecule is a small molecule of formula (II) wherein:
(i) R₁, R₂ and R₃ are selected from hydrogen, fluoro, chloro, bromo, hydroxy, methoxy, ethoxy, n-propyloxy, iso-propyloxy, n-butoxy, trifluoromethoxy, difluoromethoxy, fluoromethoxy, amino, N-methylamino, N-ethylamino, N—N-propylamino, and N,N-dimethylamino. In some aspects, R₁, R₂ and R₃ are selected from hydrogen, fluoro, chloro, bromo, hydroxy, methoxy, ethoxy, n-propyloxy, amino, N-methylamino, N-ethylamino, N—N-propylamino, and N,N-dimethylamino;
(ii) Z is selected from a straight chain or branched (C₁-C₈)alkyl, a straight chain or branched (C₂-C₈)alkenyl, a straight chain or branched (C₂-C₈)alkynyl, and —CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy; and
(iii) L is a double bond.

In some aspects, the LRRC4 family mimic molecule is a small molecule of formula (ii) wherein:
(i) R₁, R₂ and R₃ are selected from hydrogen, fluoro, chloro, bromo, hydroxy, methoxy, ethoxy, n-propyloxy, amino, N-methylamino, N-ethylamino, N—N-propylamino, and N,N-dimethylamino;
(ii) Z is selected from a straight chain or branched (C₁-C₈)alkyl, a straight chain or branched (C₂-C₈)alkenyl, and —CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxy; and
(iii) L is a double bond.

In some aspects, the LRRC4 family molecule is selected from:

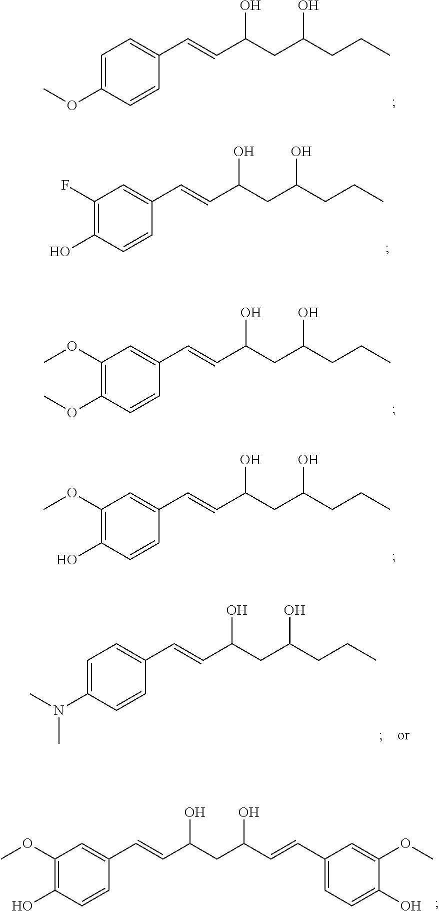

or a pharmaceutically acceptable salt thereof.

In some aspects, the LRRC4 family mimic molecule is:

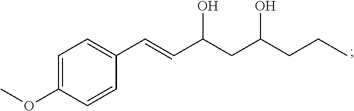 [SMILES: CCCC(O)CC(O)/C=C/c1ccc(OC)cc1]

or a pharmaceutically acceptable salt thereof.

In some aspects, the LRRC4 family mimic molecule is:

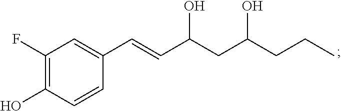 [SMILES: CCCC(O)CC(O)/C=C/c1ccc(O)c(F)c1]

or a pharmaceutically acceptable salt thereof.

In some aspects, the LRRC4 family mimic molecule is:

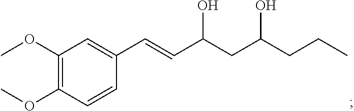 [SMILES: CCCC(O)CC(O)/C=C/c1ccc(OC)c(OC)c1]

or a pharmaceutically acceptable salt thereof.

In some aspects, the LRRC4 family mimic molecule is:

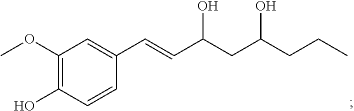 [SMILES: CCCC(O)CC(O)/C=C/c1ccc(O)c(OC)c1]

or a pharmaceutically acceptable salt thereof.

In some aspects, the LRRC4 family mimic molecule is:

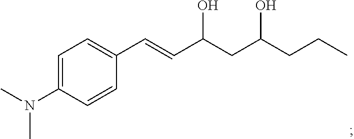 [SMILES: CCCC(O)CC(O)/C=C/c1ccc(N(C)C)cc1]

or a pharmaceutically acceptable salt thereof.

In some aspects, the LRRC4 family mimic molecule is:

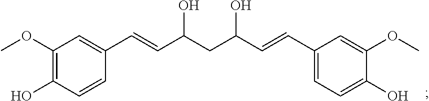

or a pharmaceutically acceptable salt thereof.

In some aspects, the LRRC4 family mimic molecule is:

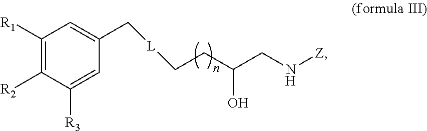

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy,
(iii) L is single, double or triple bond, and
(iv) n is 0 or 1.

In some aspects, R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy. In some aspects, R₁, R₂ and R₃ are selected from hydrogen, hydroxy, methoxy, ethoxy, n-propyloxy, iso-propyloxy, n-butoxy. In some aspects, R₁, R₂ and R₃ are selected from hydrogen, hydroxy, and methoxy. In some aspects, R₁ and R₂ are selected from hydroxy and methoxy and R3 is hydrogen.

In some aspects, Z is selected from —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy. In some aspects, Z is selected from —Y—(C₆-C₁₀)aryl, and —Y-(5-10-membered) heteroaryl wherein Y is a bond or a C₁-C₃ straight or branched alkylene, and wherein the aryl and the heteroaryl are optionally substituted with one, two, or three substituents independently selected from C₁-C₆alkoxy, and halo.

In some aspects, the LRRC4 family mimic molecule is a small molecule of formula (III) wherein:
(i) R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy;
(ii) Z is selected from —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy; and
(iii) L is a triple bond.

In some aspects, the LRRC4 family mimic molecule is a small molecule of formula (III) wherein:
(i) R₁, R₂ and R₃ are selected from hydrogen, hydroxy, methoxy, ethoxy, n-propyloxy, iso-propyloxy, and n-butoxy;
(ii) Z is selected from —Y—(C₆-C₁₀)aryl, and —Y-(5-10-membered) heteroaryl wherein Y is a bond or a C₁-C₃ straight or branched alkylene, and wherein the aryl and the heteroaryl are optionally substituted with one, two, or three substituents independently selected from C₁-C₆alkoxy, and halo; and
(iii) L is a triple bond.

In some aspects, the LRRC4 family mimic molecule is selected from:

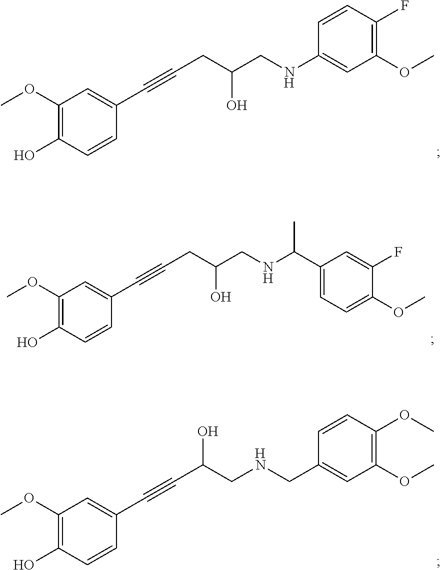

or a pharmaceutically acceptable salt thereof.

In some aspects, the LRRC4 family mimic molecule is:

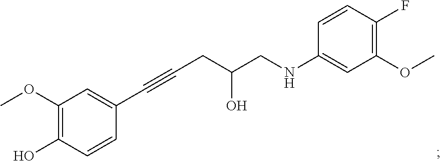

or a pharmaceutically acceptable salt thereof.

In some aspects, the LRRC4 family mimic molecule is:

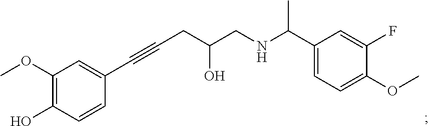

or a pharmaceutically acceptable salt thereof.

In some aspects, the LRRC4 family mimic molecule is:

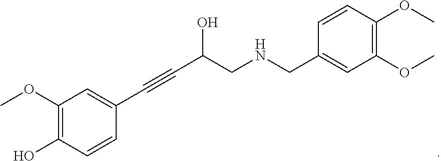

As described herein, where the LRRC4 family mimic molecule useful for the present disclosure comprises a polypeptide, in some aspects, the polypeptide comprises at least the FAM19A5 binding domain of members of the LRRC4 protein family. Unless indicated otherwise, the overall length of the FAM19A5 binding domain is not particularly limited, as long as the domain is capable of binding to the FAM19A5 protein. In some aspects, the FAM19A5 binding domain is at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 amino acids in length. In some aspects, the FAM19A5 binding domain is about 10 to about 23 amino acids in length. In some aspects, the FAM19A5 binding domain is about 10, about 11, about 12, about 13, about 14, about 15, about 16, about 17, about 18, about 19, about 20, about 21, about 22, or about 23 amino acids in length. In some aspects, the FAM19A5 binding domain of a LRRC4 family mimic molecule is about 10 amino acids in length.

In some aspects, the polypeptide of a LRRC4 family mimic molecule comprises an amino acid sequence having the following formula (from N-terminus to C-terminus):

A-(T/S)-B (SEQ ID NO: 25), wherein: (Formula IV)
(i) “A” comprises X1-(T/S)-(Y/F)—F-X5, and (ii) “B” comprises (V/I)-T-V-(E/V), and wherein:
X1 is tyrosine (Y), phenylalanine (F), valine (V), leucine (L), or isoleucine (I);
(T/S) is threonine (T) or serine (S);
(Y/F) is tyrosine (Y) or Phenylalanine (F);
X5 is any amino acids;
(V/I) is valine (V) or isoleucine (I); and
(E/V) is glutamic acid (E) or valine (V).

In some aspects, the polypeptide of a LRRC4 family mimic molecule described herein comprises an amino acid sequence having the formula (from N-terminus to C-terminus):

A-(T/S)-B (SEQ ID NO: 26), wherein: (Formula IV)
(i) “A” comprises (Y/W/M)-(T/Y)-(Y/W)-(F/Y/W)-(T/Y), and (ii) “B” comprises X7-(T/S/Y)-X9-X10, and wherein
(Y/W/M) is tyrosine (Y), tryptophan (W), or methionine (M);
(T/Y) is threonine (T) or tyrosine (Y);
(Y/W) is tyrosine (Y) or tryptophan (W);
(F/Y/W) is phenylalanine (F), tyrosine (Y), or tryptophan (W);
X7 is valine (V), tyrosine (Y), phenylalanine (F), leucine (L), tryptophan (W), or methionine (M);
(T/S/Y) is threonine (T), serine (S), or tyrosine (Y);
X9 is valine (V), isoleucine (I), tyrosine (Y), phenylalanine (F), leucine (L), tryptophan
(W), or methionine (M); and
X10 is glutamic acid (E), aspartic acid (D), isoleucine (I), tyrosine (Y), phenylalanine (F), methionine (M), or tryptophan (W).

In some aspects, a LRRC4 family mimic molecule useful for the present disclosure comprises a polypeptide that comprises an amino acid sequence having the following formula (from N-terminus to C-terminus):

X1-X2-X3-F-X5-T-X7-T-V-X10 (SEQ ID NO: 27), wherein: (Formula V)
X1 is Y, F, V, L, or I;
X2 is T or S;
X3 is Y or F;
X5 is any amino acid;
X7 is V or I; and/or
X10 is E or V,
   and wherein the polypeptide is capable of binding to a FAM19A5 protein, and thereby inhibiting, reducing, and/or dissociating the interaction between the FAM19A5 protein and members of the LRRC4 protein family.

In some aspects, a LRRC4 family mimic molecule described herein comprises a polypeptide that comprises an amino acid sequence having the following formula (from N-terminus to C-terminus):

X1-X2-X3-X4-X5-X6-X7-X8-X9-X10 (SEQ ID NO: 28), wherein: (Formula VI)
X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W,
   and wherein the polypeptide is capable of binding to a FAM19A5 protein, and thereby inhibiting, reducing, and/or dissociating the interaction between the FAM19A5 protein and members of the LRRC4 protein family.

For any of the above LRRC4 family mimic molecules, in some aspects, (i) X1 is Y, F, V, L, or I; (ii) X2 is T or S; (iii) X3 is Y or F; (iv) X4 is F; (v) X5 is T or S; (vi) X6 is T; (vii) X7 is V or I; (viii) X8 is T; (ix) X9 is V; (x) X10 is E or V; and (xi) any combinations of (i)-(x). In some aspects, X1 is Y, F, V, L, or I. In some aspects, X2 is T or S. In some aspects, X3 is Y or F. In some aspects, X4 is F. In some aspects, X5 is T or S. In some aspects, X6 is T. In some aspects, X7 is V or I. In some aspects, X8 is T. In some aspects, X9 is V. In some aspects, X10 is E or V. In some aspects, the amino acid at position X2 is phosphorylated. In some aspects, the amino acid at position X2 is O-glycosylated.

In some aspects, a polypeptide of a LRRC4 family mimic molecule described herein comprises the amino acid sequence set forth in SEQ ID NO: 29 (YTYFTTVTVE), with one, two, three, four, five, or six amino acids different from the amino acid sequence (e.g., substitutions). In some aspects, a polypeptide of a LRRC4 family mimic molecule disclosed herein consists of the amino acid sequence set forth in SEQ ID NO: 29 (YTYFTTVTVE), with one, two, three, four, five, or six amino acids different from the amino acid sequence (e.g., substitutions). In some aspects, a polypeptide of a LRRC4 family mimic molecule disclosed herein consists essentially of the amino acid sequence set forth in SEQ ID NO: 29 (YTYFTTVTVE), with one, two, three, four, five, or six amino acids different from the amino acid sequence (e.g., substitutions).

In some aspects, a polypeptide of a LRRC4 family mimic molecule comprises the amino acid sequence set forth in SEQ ID NO: 29 (YTYFTTVTVE). In some aspects, the polypeptide of a LRRC4 family mimic molecule consists of the amino acid sequence set forth in SEQ ID NO: 29 (YTYFTTVTVE). In some aspects, the polypeptide of a LRRC4 family mimic molecule consists essentially of the amino acid sequence set forth in SEQ ID NO: 29 (YTYFTTVTVE). As demonstrated herein, the amino acid sequence set forth in SEQ ID NO: 29 (YTYFTTVTVE) corresponds to the FAM19A5 binding domain of the LRRC4B protein.

In some aspects, a polypeptide of a LRRC4 family mimic molecule comprises the amino acid sequence set forth in SEQ ID NO: 30 (YSFFTTVTVE). In some aspects, the polypeptide of a LRRC4 family mimic molecule consists of the amino acid sequence set forth in SEQ ID NO: 30 (YSFFTTVTVE). In some aspects, the polypeptide of a LRRC4 family mimic molecule consists essentially of the amino acid sequence set forth in SEQ ID NO: 30 (YSFFTTVTVE). As demonstrated herein, the amino acid sequence set forth in SEQ ID NO: 30 (YSFFTTVTVE) corresponds to the FAM19A5 binding domain of the LRRC4 protein.

In some aspects, a polypeptide of a LRRC4 family mimic molecule comprises the amino acid sequence set forth in SEQ ID NO: 31 (FSYFSTVTVE). In some aspects, the polypeptide of a LRRC4 family mimic molecule consists of the amino acid sequence set forth in SEQ ID NO: 31 (FSYFSTVTVE). In some aspects, the polypeptide of a LRRC4 family mimic molecule consists essentially of the amino acid sequence set forth in SEQ ID NO: 31 (FSYFSTVTVE). As demonstrated herein, the amino acid sequence set forth in SEQ ID NO: 31 (FSYFSTVTVE) corresponds to the FAM19A5 binding domain of the LRRC4C protein.

As described herein, the FAM19A5 binding domains of members of the LRRC4 protein family are largely conserved among vertebrates (see, e.g., FIG. 20). Accordingly, not to be bound by any one theory, one or more amino acid residues of the amino acid sequence set forth in any one of SEQ ID NOs: 29 (YTYFTTVTVE), 30 (YSFFTTVTVE), and 31 (FSYFSTVTVE) can be substituted with an amino acid present in the corresponding residue in other vertebrates. Examples of such substitutions are provided herein (see, e.g., FIG. 20).

In some aspects, one or more amino acid residues of the amino acid sequence set forth in any one of SEQ ID NOs: 29 (YTYFTTVTVE), 30 (YSFFTTVTVE), and 31 (FSYFSTVTVE) can be substituted with an amino acid sharing similar biochemical properties. For instance, in the amino acid sequence set forth in SEQ ID NO: 29 (YTYFTTVTVE), in some aspects, the Y at position 1 can be substituted with other hydrophobic amino acids (e.g., F, V, L, I, W, or M). In some aspects, the T at position 2 can be substituted with other amino acids having a similar hydroxyl (OH) group in its side chain (e.g., S or Y). In some aspects, the Y at position 3 can be substituted with other amino acids having common aromatic ring in its side chain that can participate in Van der Waals interaction (e.g., F or W). In some aspects, the F at position 4 can be substituted with amino acids, such as Y or W. In some aspects, the T at position 5 can be substituted with amino acids, such as S or Y. In some aspects, the T at position 6 can be substituted with amino acids, such as S or Y. In some aspects, the V at position 7 can be substituted with other amino acids having hydrophobic bulky side chains (e.g., I, Y, F, L, W, or M). In some aspects, the T at position 8 can be substituted with other amino acids, such as S or Y. In some aspects, the V at position 9 can be substituted with other amino acids, such as I, Y, F, L, W, or M. In some aspects, the E at position 10 can be substituted with other amino acids that have an acidic side chain (e.g., I, Y, F, M, or W).

In some aspects, a polypeptide of a LRRC4 family mimic molecule described herein comprises an amino acid sequence that is at least about 40%, at least about 50%, at least about 60%, at least about 70%, at least about 80%, or at least about 90% identical to the amino acid sequence set forth in SEQ ID NO: 29 (YTYFTTVTVE), wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 5, wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 4, wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 6, wherein the polypeptide is capable of binding to a FAM19A5 protein, and thereby inhibiting, reducing, and/or dissociating the interaction between the FAM19A5 protein and members of the LRRC4 protein family.

As is apparent from the present disclosure, in some aspects, polypeptides of the LRRC4 family mimic molecules described herein (e.g., comprising a FAM19A5 binding domain of members of the LRRC4 protein family) comprise one or more amino acid modifications. In some aspects, the one or more amino acid modifications can increase the binding affinity of the LRRC4 family mimic molecule to the FAM19A5 protein. Accordingly, in some aspects, the binding affinity of a LRRC4 family mimic molecule described herein to a FAM19A5 protein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family). In some aspects, the one or more amino acid modifications can improve the stability of the LRRC4 family mimic molecule. Accordingly, in some aspects, the stability of a LRRC4 family mimic molecule described herein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family).

In some aspects, the one or more amino acid modifications can improve the ability of the LRRC4 family mimic molecules described herein to inhibit the interaction between a FAM19A5 protein and members of the LRRC4 protein family (e.g., by increasing the binding affinity and/or stability). Accordingly, in some aspects, the ability of the LRRC4 family mimic molecule to inhibit the interaction between a FAM19A5 protein and members of the LRRC4 protein family is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family).

Non-limiting examples of amino acid modifications that are useful for the present disclosure are provided throughout the present disclosure. For instance, in some aspects, a LRRC4 family mimic molecule described herein comprises one of the FAM19A5 binding domains of a members of the LRRC4 protein family—i.e., YTYFTTVTVE (SEQ ID NO: 29), YSFFTTVTVE (SEQ ID NO: 30), or FSYFSTVTVE (SEQ ID NO: 31)—and one or more amino acids at the N-terminus, C-terminus, or both at the N-terminus and C-terminus of the polypeptide. In some aspects, a polypeptide useful for the present disclosure comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide. In some aspects, the polypeptide comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about additional 20 amino acids at the C-terminus of the polypeptide. In some aspects, the polypeptide comprises: (i) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide; and (ii) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the C-terminus of the polypeptide. As demonstrated herein (see, e.g., Example 9), in some aspects, the one or more amino acids differ from the amino acids present at the particular residues in a naturally existing LRRC4 protein family member.

For instance, in some aspects, a polypeptide described herein comprises the amino acid sequence set forth in SEQ ID NO: 18 (GYTYFTTVTVETLETQPGEE) with one or more amino acid modifications (e.g., substitutions). In some aspects, a polypeptide described herein comprises the amino acid sequence set forth in SEQ ID NO: 18 (GYTYFTTVTVETLETQPGEE) with two amino acid modifications (e.g., substitutions). In some aspects, the amino acid modifications are at residues T12 and L13 of SEQ ID NO: 18. In some aspects, a polypeptide described herein consists of the amino acid sequence set forth in SEQ ID NO: 18 (GYTYFTTVTVETLETQPGEE) with one or more amino acid modifications (e.g., substitutions). In some aspects, a polypeptide described herein consists of the amino acid sequence set forth in SEQ ID NO: 18 (GYTYFTTVTVETLETQPGEE) with two amino acid modifications (e.g., substitutions). In some aspects, the amino acid modifications are at residues T12 and L13 of SEQ ID NO: 18. In some aspects, a polypeptide described herein consists essentially of the amino acid sequence set forth in SEQ ID NO: 18 (GYTYFTTVTVETLETQPGEE) with one or more amino acid modifications (e.g., substitutions). In some aspects, a polypeptide described herein consists essentially of the amino acid sequence set forth in SEQ ID NO: 18 (GYTYFTTVTVETLETQPGEE) with two amino acid modifications (e.g., substitutions). In some aspects, the amino acid modifications are at residues T12 and L13 of SEQ ID NO: 18.

In some aspects, a polypeptide described herein comprises the amino acid sequence set forth in SEQ ID NO: 17 (GYTYFTTVTVETLETQ) with one or more amino acid modifications (e.g., substitutions). In some aspects, a polypeptide described herein comprises the amino acid sequence set forth in SEQ ID NO: 17 (GYTYFTTVTVETLETQ) with two amino acid modifications (e.g., substitutions). In some aspects, the amino acid modifications are at residues T12 and L13 of SEQ ID NO: 17. In some aspects, a polypeptide described herein consists of the amino acid sequence set forth in SEQ ID NO: 17 (GYTYFTTVTVETLETQ) with one or more amino acid modifications (e.g., substitutions). In some aspects, a polypeptide described herein consists of the amino acid sequence set forth in SEQ ID NO: 17 (GYTYFTTVTVETLETQ) with two amino acid modifications (e.g., substitutions). In some aspects, the amino acid modifications are at residues T12 and L13 of SEQ ID NO: 17. In some aspects, a polypeptide described herein consists essentially of the amino acid sequence set forth in SEQ ID NO: 17 (GYTYFTTVTVETLETQ) with one or more amino acid modifications (e.g., substitutions). In some aspects, a polypeptide described herein consists essentially of the amino acid sequence set forth in SEQ ID NO: 17 (GYTYFTTVTVETLETQ) with two amino acid modifications (e.g., substitutions). In some aspects, the amino acid modifications are at residues T12 and L13 of SEQ ID NO: 17.

In some aspects, a polypeptide described herein comprises the amino acid sequence set forth in SEQ ID NO: 19 (GYTYFTTVTVETLETQPGEKEPPGPTTD) with one or more amino acid modifications (e.g., substitutions). In some aspects, a polypeptide described herein comprises the amino acid sequence set forth in SEQ ID NO: 19 (GYTYFTTVTVETLETQPGEKEPPGPTTD) with two amino acid modifications (e.g., substitutions). In some aspects, the amino acid modifications are at residues T12 and L13 of SEQ ID NO: 19. In some aspects, a polypeptide described herein consists of the amino acid sequence set forth in SEQ ID NO: 19 (GYTYFTTVTVETLETQPGEKEPPGPTTD) with one or more amino acid modifications (e.g., substitutions). In some aspects, a polypeptide described herein consists of the amino acid sequence set forth in SEQ ID NO: 19 (GYTYFTTVTVETLETQPGEKEPPGPTTD) with two amino acid modifications (e.g., substitutions). In some aspects, the amino acid modifications are at residues T12 and L13 of SEQ ID NO: 19. In some aspects, a polypeptide described herein consists essentially of the amino acid sequence set forth in SEQ ID NO: 19 (GYTYFTTVTVETLETQPGEKEPPGPTTD) with one or more amino acid modifications (e.g., substitutions). In some aspects, a polypeptide described herein consists essentially of the amino acid sequence set forth in SEQ ID NO: 19 (GYTYFTTVTVETLETQPGEKEPPGPTTD) with two amino acid modifications (e.g., substitutions). In some aspects, the amino acid modifications are at residues T12 and L13 of SEQ ID NO: 19.

In some aspects, a polypeptide described herein comprises the amino acid sequence set forth in SEQ ID NO: 143 (GYTYFTTVTVETLETQPGEEA) with one or more amino acid modifications (e.g., substitutions). In some aspects, a polypeptide described herein comprises the amino acid sequence set forth in SEQ ID NO: 143 (GYTYFTTVTVETLETQPGEEA) with two amino acid modifications (e.g., substitutions). In some aspects, the amino acid modifications are at residues T12 and L13 of SEQ ID NO: 143. In some aspects, a polypeptide described herein consists of the amino acid sequence set forth in SEQ ID NO: 143 (GYTYFTTVTVETLETQPGEEA) with one or more amino acid modifications (e.g., substitutions). In some aspects, a polypeptide described herein consists of the amino acid sequence set forth in SEQ ID NO: 143 (GYTYFTTVTVETLETQPGEEA) with two amino acid modifications (e.g., substitutions). In some aspects, the amino acid modifications are at residues T12 and L13 of SEQ ID NO: 143. In some aspects, a polypeptide described herein consists essentially of the amino acid sequence set forth in SEQ ID NO: 143 (GYTYFTTVTVETLETQPGEEA) with one or more amino acid modifications (e.g., substitutions). In some aspects, a polypeptide described herein consists essentially of the amino acid sequence set forth in SEQ ID NO: 143 (GYTYFTTVTVETLETQPGEEA) with two amino acid modifications (e.g., substitutions). In some aspects, the amino acid modifications are at residues T12 and L13 of SEQ ID NO: 143.

In some aspects, a polypeptide described herein (e.g., comprising the FAM19A5 binding domain of LRRC4B) comprises the amino acid sequence GYTYFTTVTVEPYETQPGEE (SEQ ID NO: 123). In some aspects, a polypeptide described herein (e.g., comprising the FAM19A5 binding domain of LRRC4B) consists of the amino acid sequence GYTYFTTVTVEPYETQPGEE (SEQ ID NO: 123). In some aspects, a polypeptide described herein (e.g., comprising the FAM19A5 binding domain of LRRC4B) consists essentially of the amino acid sequence GYTYFTTVTVEPYETQPGEE (SEQ ID NO: 123).

In some aspects, a polypeptide described herein (e.g., comprising the FAM19A5 binding domain of LRRC4B) comprises the amino acid sequence GYTYFTTVTVEMRETQPGEE (SEQ ID NO: 124). In some aspects, a polypeptide described herein (e.g., comprising the FAM19A5 binding domain of LRRC4B) consists of the amino acid sequence GYTYFTTVTVEMRETQPGEE (SEQ ID NO: 124). In some aspects, a polypeptide described herein (e.g., comprising the FAM19A5 binding domain of LRRC4B) consists essentially of the amino acid sequence GYTYFTTVTVEMRETQPGEE (SEQ ID NO: 124).

In some aspects, a polypeptide described herein (e.g., comprising the FAM19A5 binding domain of LRRC4B) comprises the amino acid sequence GYTYFTTVTVEIFETQPGEE (SEQ ID NO: 125). In some aspects, a polypeptide described herein (e.g., comprising the FAM19A5 binding domain of LRRC4B) consists of the amino acid sequence GYTYFTTVTVEIFETQPGEE (SEQ ID NO: 125). In some aspects, a polypeptide described herein (e.g., comprising the FAM19A5 binding domain of LRRC4B) consists essentially of the amino acid sequence GYTYFTTVTVEIFETQPGEE (SEQ ID NO: 125).

In some aspects, a polypeptide described herein (e.g., comprising the FAM19A5 binding domain of LRRC4B) comprises the amino acid sequence GYTYFTTVTVEHFETQPGEE (SEQ ID NO: 126). In some aspects, a polypeptide described herein (e.g., comprising the FAM19A5 binding domain of LRRC4B) consists of the amino acid sequence GYTYFTTVTVEHFETQPGEE (SEQ ID NO: 126). In some aspects, a polypeptide described herein (e.g., comprising the FAM19A5 binding domain of LRRC4B) consists essentially of the amino acid sequence GYTYFTTVTVEHFETQPGEE (SEQ ID NO: 126).

In some aspects, a polypeptide described herein (e.g., comprising the FAM19A5 binding domain of LRRC4B) comprises the amino acid sequence GYTYFTTVTVEWYETQPGEE (SEQ ID NO: 127). In some aspects, a polypeptide described herein (e.g., comprising the FAM19A5 binding domain of LRRC4B) consists of the amino acid sequence GYTYFTTVTVEWYETQPGEE (SEQ ID NO: 127). In some aspects, a polypeptide described herein (e.g., comprising the FAM19A5 binding domain of LRRC4B) consists essentially of the amino acid sequence GYTYFTTVTVEWYETQPGEE (SEQ ID NO: 127).

In some aspects, a polypeptide described herein (e.g., comprising the FAM19A5 binding domain of LRRC4B) comprises the amino acid sequence GYTYFTTVTVEQRETQPGEE (SEQ ID NO: 128). In some aspects, a polypeptide described herein (e.g., comprising the FAM19A5 binding domain of LRRC4B) consists of the amino acid sequence GYTYFTTVTVEQRETQPGEE (SEQ ID NO: 128). In some aspects, a polypeptide described herein (e.g., comprising the FAM19A5 binding domain of LRRC4B) consists essentially of the amino acid sequence GYTYFTTVTVEQRETQPGEE (SEQ ID NO: 128).

In some aspects, a polypeptide described herein (e.g., comprising the FAM19A5 binding domain of LRRC4B) comprises the amino acid sequence GYTYFTTVTVEWFETQPGEE (SEQ ID NO: 129). In some aspects, a polypeptide described herein (e.g., comprising the FAM19A5 binding domain of LRRC4B) consists of the amino acid sequence GYTYFTTVTVEWFETQPGEE (SEQ ID NO: 129). In some aspects, a polypeptide described herein (e.g., comprising the FAM19A5 binding domain of LRRC4B) consists essentially of the amino acid sequence GYTYFTTVTVEWFETQPGEE (SEQ ID NO: 129).

In some aspects, a polypeptide described herein (e.g., comprising the FAM19A5 binding domain of LRRC4B) comprises the amino acid sequence GYTYFTTVTVEERETQPGEE (SEQ ID NO: 130). In some aspects, a polypeptide described herein (e.g., comprising the FAM19A5 binding domain of LRRC4B) consists of the amino acid sequence GYTYFTTVTVEERETQPGEE (SEQ ID NO: 130). In some aspects, a polypeptide described herein (e.g., comprising the FAM19A5 binding domain of LRRC4B) consists essentially of the amino acid sequence GYTYFTTVTVEERETQPGEE (SEQ ID NO: 130).

In some aspects, a polypeptide described herein (e.g., comprising the FAM19A5 binding domain of LRRC4B) comprises the amino acid sequence GYTYFTTVTVEDYETQPGEE (SEQ ID NO: 131). In some aspects, a polypeptide described herein (e.g., comprising the FAM19A5 binding domain of LRRC4B) consists of the amino acid sequence GYTYFTTVTVEDYETQPGEE (SEQ ID NO: 131). In some aspects, a polypeptide described herein (e.g., comprising the FAM19A5 binding domain of LRRC4B) consists essentially of the amino acid sequence GYTYFTTVTVEDYETQPGEE (SEQ ID NO: 131).

In some aspects, a polypeptide described herein (e.g., comprising the FAM19A5 binding domain of LRRC4B) comprises the amino acid sequence GYTYFTTVTVEFFETQPGEE (SEQ ID NO: 132). In some aspects, a polypeptide described herein (e.g., comprising the FAM19A5 binding domain of LRRC4B) consists of the amino acid sequence GYTYFTTVTVEFFETQPGEE (SEQ ID NO: 132). In some aspects, a polypeptide described herein (e.g., comprising the FAM19A5 binding domain of LRRC4B) consists essentially of the amino acid sequence GYTYFTTVTVEFFETQPGEE (SEQ ID NO: 132).

In some aspects, a polypeptide described herein (e.g., comprising the FAM19A5 binding domain of LRRC4B) comprises the amino acid sequence GYTYFTTVTVEHYETQPGEE (SEQ ID NO: 133). In some aspects, a polypeptide described herein (e.g., comprising the FAM19A5 binding domain of LRRC4B) consists of the amino acid sequence GYTYFTTVTVEHYETQPGEE (SEQ ID NO: 133). In some aspects, a polypeptide described herein (e.g., comprising the FAM19A5 binding domain of LRRC4B) consists essentially of the amino acid sequence GYTYFTTVTVEHYETQPGEE (SEQ ID NO: 133).

In some aspects, a polypeptide described herein (e.g., comprising the FAM19A5 binding domain of LRRC4B) comprises the amino acid sequence GYTYFTTVTVEMMETQPGEE (SEQ ID NO: 134). In some aspects, a polypeptide described herein (e.g., comprising the FAM19A5 binding domain of LRRC4B) consists of the amino acid sequence GYTYFTTVTVEMMETQPGEE (SEQ ID NO: 134). In some aspects, a polypeptide described herein (e.g., comprising the FAM19A5 binding domain of LRRC4B) consists essentially of the amino acid sequence GYTYFTTVTVEMMETQPGEE (SEQ ID NO: 134).

In some aspects, a polypeptide described herein (e.g., comprising the FAM19A5 binding domain of LRRC4B) comprises the amino acid sequence GYTYFTTVTVEDFETQPGEE (SEQ ID NO: 135). In some aspects, a polypeptide described herein (e.g., comprising the FAM19A5 binding domain of LRRC4B) consists of the amino acid sequence GYTYFTTVTVEDFETQPGEE (SEQ ID NO: 135). In some aspects, a polypeptide described herein (e.g., comprising the FAM19A5 binding domain of LRRC4B) consists essentially of the amino acid sequence GYTYFTTVTVEDFETQPGEE (SEQ ID NO: 135).

In some aspects, a polypeptide described herein (e.g., comprising the FAM19A5 binding domain of LRRC4B) comprises the amino acid sequence GYTYFTTVTVEDIETQPGEE (SEQ ID NO: 136). In some aspects, a polypeptide described herein (e.g., comprising the FAM19A5 binding domain of LRRC4B) consists of the amino acid sequence GYTYFTTVTVEDIETQPGEE (SEQ ID NO: 136). In some aspects, a polypeptide described herein (e.g., comprising the FAM19A5 binding domain of LRRC4B) consists essentially of the amino acid sequence GYTYFTTVTVEDIETQPGEE (SEQ ID NO: 136).

In some aspects, a polypeptide described herein (e.g., comprising the FAM19A5 binding domain of LRRC4B) comprises the amino acid sequence GYTYFTTVTVELIETQPGEE (SEQ ID NO: 137). In some aspects, a polypeptide described herein (e.g., comprising the FAM19A5 binding domain of LRRC4B) consists of the amino acid sequence GYTYFTTVTVELIETQPGEE (SEQ ID NO: 137). In some aspects, a polypeptide described herein (e.g., comprising the FAM19A5 binding domain of LRRC4B) consists essentially of the amino acid sequence GYTYFTTVTVELIETQPGEE (SEQ ID NO: 137).

In some aspects, a polypeptide described herein (e.g., comprising the FAM19A5 binding domain of LRRC4B) comprises the amino acid sequence GYTYFTTVTVEEIETQPGEE (SEQ ID NO: 138). In some aspects, a polypeptide described herein (e.g., comprising the FAM19A5 binding domain of LRRC4B) consists of the amino acid sequence GYTYFTTVTVEEIETQPGEE (SEQ ID NO: 138). In some aspects, a polypeptide described herein (e.g., comprising the FAM19A5 binding domain of LRRC4B) consists essentially of the amino acid sequence GYTYFTTVTVEEIETQPGEE (SEQ ID NO: 138).

In some aspects, a polypeptide described herein (e.g., comprising the FAM19A5 binding domain of LRRC4B) comprises the amino acid sequence GYTYFTTVTVEAFETQPGEE (SEQ ID NO: 139). In some aspects, a polypeptide described herein (e.g., comprising the FAM19A5 binding domain of LRRC4B) consists of the amino acid sequence GYTYFTTVTVEAFETQPGEE (SEQ ID NO: 139). In some aspects, a polypeptide described herein (e.g., comprising the FAM19A5 binding domain of LRRC4B) consists essentially of the amino acid sequence GYTYFTTVTVEAFETQPGEE (SEQ ID NO: 139).

In some aspects, a polypeptide described herein (e.g., comprising the FAM19A5 binding domain of LRRC4B) comprises the amino acid sequence GYTYFTTVTVEHHETQPGEE (SEQ ID NO: 140). In some aspects, a polypeptide described herein (e.g., comprising the FAM19A5 binding domain of LRRC4B) consists of the amino acid sequence GYTYFTTVTVEHHETQPGEE (SEQ ID NO: 140). In some aspects, a polypeptide described herein (e.g., comprising the FAM19A5 binding domain of LRRC4B) consists essentially of the amino acid sequence GYTYFTTVTVEHHETQPGEE (SEQ ID NO: 140).

In some aspects, a polypeptide described herein (e.g., comprising the FAM19A5 binding domain of LRRC4B) comprises the amino acid sequence GYTYFTTVTVEPFETQPGEE (SEQ ID NO: 141). In some aspects, a polypeptide described herein (e.g., comprising the FAM19A5 binding domain of LRRC4B) consists of the amino acid sequence GYTYFTTVTVEPFETQPGEE (SEQ ID NO: 141). In some aspects, a polypeptide described herein (e.g., comprising the FAM19A5 binding domain of LRRC4B) consists essentially of the amino acid sequence GYTYFTTVTVEPFETQPGEE (SEQ ID NO: 141).

In some aspects, a polypeptide described herein (e.g., comprising the FAM19A5 binding domain of LRRC4B) comprises the amino acid sequence GYTYFTTVTVEDWETQPGEE (SEQ ID NO: 142). In some aspects, a polypeptide described herein (e.g., comprising the FAM19A5 binding domain of LRRC4B) consists of the amino acid sequence GYTYFTTVTVEDWETQPGEE (SEQ ID NO: 142). In some aspects, a polypeptide described herein (e.g., comprising the FAM19A5 binding domain of LRRC4B) consists essentially of the amino acid sequence GYTYFTTVTVEDWETQPGEE (SEQ ID NO: 142).

In some aspects, a polypeptide described herein (e.g., comprising the FAM19A5 binding domain of LRRC4B) comprises the amino acid sequence GYTYFTTVTVEPYETQPGEEA (SEQ ID NO: 144). In some aspects, a polypeptide described herein (e.g., comprising the FAM19A5 binding domain of LRRC4B) consists of the amino acid sequence GYTYFTTVTVEPYETQPGEEA (SEQ ID NO: 144). In some aspects, a polypeptide described herein (e.g., comprising the FAM19A5 binding domain of LRRC4B) consists essentially of the amino acid sequence GYTYFTTVTVEPYETQPGEEA (SEQ ID NO: 144).

In some aspects, a polypeptide described herein (e.g., comprising the FAM19A5 binding domain of LRRC4B) comprises the amino acid sequence GYTYFTTVTVEHFETQPGEEA (SEQ ID NO: 145). In some aspects, a polypeptide described herein (e.g., comprising the FAM19A5 binding domain of LRRC4B) consists of the amino acid sequence GYTYFTTVTVEHFETQPGEEA (SEQ ID NO: 145). In some aspects, a polypeptide described herein (e.g., comprising the FAM19A5 binding domain of LRRC4B) consists essentially of the amino acid sequence GYTYFTTVTVEHFETQPGEEA (SEQ ID NO: 145).

In some aspects, a polypeptide described herein (e.g., comprising the FAM19A5 binding domain of LRRC4B) comprises the amino acid sequence GYTYFTTVTVEQRETQPGEEA (SEQ ID NO: 146). In some aspects, a polypeptide described herein (e.g., comprising the FAM19A5 binding domain of LRRC4B) consists of the amino acid sequence GYTYFTTVTVEQRETQPGEEA (SEQ ID NO: 146). In some aspects, a polypeptide described herein (e.g., comprising the FAM19A5 binding domain of LRRC4B) consists essentially of the amino acid sequence GYTYFTTVTVEQRETQPGEEA (SEQ ID NO: 146).

In some aspects, a polypeptide described herein (e.g., comprising the FAM19A5 binding domain of LRRC4B) comprises the amino acid sequence GYTYFTTVTVEWYETQPGEEA (SEQ ID NO: 147). In some aspects, a polypeptide described herein (e.g., comprising the FAM19A5 binding domain of LRRC4B) consists of the amino acid sequence GYTYFTTVTVEWYETQPGEEA (SEQ ID NO: 147). In some aspects, a polypeptide described herein (e.g., comprising the FAM19A5 binding domain of LRRC4B) consists essentially of the amino acid sequence GYTYFTTVTVEWYETQPGEEA (SEQ ID NO: 147).

In some aspects, a polypeptide described herein (e.g., comprising the FAM19A5 binding domain of LRRC4B) comprises the amino acid sequence GYTYFTTVTVEMRETQPGEEA (SEQ ID NO: 148). In some aspects, a polypeptide described herein (e.g., comprising the FAM19A5 binding domain of LRRC4B) consists of the amino acid sequence GYTYFTTVTVEMRETQPGEEA (SEQ ID NO: 148). In some aspects, a polypeptide described herein (e.g., comprising the FAM19A5 binding domain of LRRC4B) consists essentially of the amino acid sequence GYTYFTTVTVEMRETQPGEEA (SEQ ID NO: 148).

In some aspects, a polypeptide described herein (e.g., comprising the FAM19A5 binding domain of LRRC4B) comprises the amino acid sequence GYTYFTTVTVEIFETQPGEEA (SEQ ID NO: 149). In some aspects, a polypeptide described herein (e.g., comprising the FAM19A5 binding domain of LRRC4B) consists of the amino acid sequence GYTYFTTVTVEIFETQPGEEA (SEQ ID NO: 149). In some aspects, a polypeptide described herein (e.g., comprising the FAM19A5 binding domain of LRRC4B) consists essentially of the amino acid sequence GYTYFTTVTVEIFETQPGEEA (SEQ ID NO: 149).

In some aspects, a LRRC4 family mimic molecule described herein comprises one or more components that can improve the ability of the polypeptide to inhibit the interaction between a FAM19A5 protein and members of the LRRC4 protein family. For instance, in some aspects, a molecule comprises (i) any of the polypeptides described herein and (ii) one or more additional amino acids at the N-terminus of the polypeptide, the C-terminus of the polypeptide, or both the N-terminus and the C-terminus of the polypeptide. In some aspects, a molecule useful for the present disclosure comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide. In some aspects, a molecule comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about additional 20 amino acids at the C-terminus of the polypeptide. In some aspects, a molecule comprises: (i) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide; and (ii) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the C-terminus of the polypeptide.

In some aspects, a LRRC4 family mimic molecule comprises: (i) a polypeptide having the amino acid sequence set forth in SEQ ID NO: 29 (YTYFTTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 29 (YTYFTTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 29 (YTYFTTVTVE) and (ii) at least one additional amino acid at both the N-terminus and the C-terminus. In some aspects, a LRRC4 family mimic molecule useful for the present disclosure comprises the amino acid sequence set forth in SEQ ID NO: 18 (GYTYFTTVTVETLETQPGEE). In some aspects, a LRRC4 family mimic molecule consists of the amino acid sequence set forth in SEQ ID NO: 18 (GYTYFTTVTVETLETQPGEE). In some aspects, a LRRC4 family mimic molecule consists essentially of the amino acid sequence set forth in SEQ ID NO: 18 (GYTYFTTVTVETLETQPGEE). In some aspects, a LRRC4 family mimic molecule useful for the present disclosure comprises the amino acid sequence set forth in SEQ ID NO: 17 (GYTYFTTVTVETLETQ). In some aspects, a LRRC4 family mimic molecule consists of the amino acid sequence set forth in SEQ ID NO: 17 (GYTYFTTVTVETLETQ). In some aspects, a LRRC4 family mimic molecule consists essentially of the amino acid sequence set forth in SEQ ID NO: 17 (GYTYFTTVTVETLETQ). In some aspects, a LRRC4 family mimic molecule useful for the present disclosure comprises the amino acid sequence set forth in SEQ ID NO: 19 (GYTYFTTVTVETLETQPGEKEPPGPTTD). In some aspects, a LRRC4 family mimic molecule consists of the amino acid sequence set forth in SEQ ID NO: 19 (GYTYFTTVTVETLETQPGEKEPPGPTTD). In some aspects, a LRRC4 family mimic molecule consists essentially of the amino acid sequence set forth in SEQ ID NO: 19 (GYTYFTTVTVETLETQPGEKEPPGPTTD).

In some aspects, a LRRC4 family mimic molecule useful for the present disclosure comprises: (i) a polypeptide having the amino acid sequence set forth in SEQ ID NO: 30 (YSFFTTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 30 (YSFFTTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 30 (YSFFTTVTVE) and (ii) at least one additional amino acid at both the N-terminus and C-terminus. In some aspects, a LRRC4 family mimic molecule useful for the present disclosure comprises the amino acid sequence set forth in SEQ ID NO: 20 (NYSFFTTVTVETTEISPEDTTRK). In some aspects, a LRRC4 family mimic molecule consists of the amino acid sequence set forth in SEQ ID NO: 20 (NYSFFTTVTVETTEISPEDTTRK). In some aspects, a LRRC4 family mimic molecule consists essentially of the amino acid sequence set forth in SEQ ID NO: 20 (NYSFFTTVTVETTEISPEDTTRK).

In some aspects, a LRRC4 family mimic molecule useful for the present disclosure comprises: (i) a polypeptide having the amino acid sequence set forth in SEQ ID NO: 31 (FSYFSTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 31 (FSYFSTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 31 (FSYFSTVTVE) and (ii) at least one additional amino acid at both the N-terminus and the C-terminus. In some aspects, a LRRC4 family mimic molecule useful for the present disclosure comprises the amino acid sequence set forth in SEQ ID NO: 21 (NFSYFSTVTVETMEPSQDERTTR). In some aspects, a LRRC4 family mimic molecule consists of the amino acid sequence set forth in SEQ ID NO: 21 (NFSYFSTVTVETMEPSQDERTTR). In some aspects, a LRRC4 family mimic molecule consists essentially of the amino acid sequence set forth in SEQ ID NO: 21 (NFSYFSTVTVETMEPSQDERTTR).

In some aspects, a polypeptide of a LRRC4 family mimic molecule comprises an amino acid sequence that is at least about 40%, at least about 50%, at least about 60%, at least about 70%, at least about 80%, or at least about 90% identical to the amino acid sequence set forth in SEQ ID NO: 29 (YTYFTTVTVE), wherein the polypeptide is capable of binding to a FAM19A5 protein and wherein the amino acid sequence further comprises one or more hydrophobic amino acids at the N-terminus. In some aspects, the hydrophobic amino acids comprise at least two amino acids, at least three amino acids, at least four amino acids, at least five amino acids, at least six amino acids, at least seven amino acids, at least eight amino acids, at least nine amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the N terminus.

In some aspects, a polypeptide of a LRRC4 family mimic molecule comprises an amino acid sequence that is at least about 40%, at least about 50%, at least about 60%, at least about 70%, at least about 80%, or at least about 90% identical to the amino acid sequence set forth in SEQ ID NO: 29 (YTYFTTVTVE), wherein the polypeptide is capable of binding to a FAM19A5 protein and wherein the amino acid sequence further comprises at the N-terminus and/or at the-C terminus one or more amino acids. In some aspects, the one or more amino acids linked to the N-terminus and/or C-terminus comprise one or more amino acid sequences derived from a LRRC4B protein. In some aspects, the one or more amino acids linked to the N-terminus comprises at least two amino acids, at least three amino acids, at least four amino acids, at least five amino acids, at least six amino acids, at least seven amino acids, at least eight amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the N-terminus. In some aspects, the one or more amino acids linked to the C-terminus comprise at least two amino acids, at least three amino acids, at least four amino acids, at least five amino acids, at least six amino acids, at least seven amino acids, at least eight amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the C-terminus. In some aspects, the one or more amino acids linked to the N-terminus and/or C-terminus are linked via a linker. In some aspects, the linker is a peptide linker.

In some aspects, the one or more additional amino acids that are added at the N-terminus and/or the C-terminus can comprise any suitable amino acids known in the art. In some aspects, the one or more additional amino acids are hydrophilic amino acids. In some aspects, the one or more additional amino acids can comprise D-amino acids. Not to be bound by any one theory, in some aspects, the addition of one or more D-amino acids at the N-terminus and/or the C-terminus of the polypeptide can enhance the persistence of the LRRC4 family mimic molecule, e.g., when administered to a subject. For instance, the inclusion of the D-amino acids can protect the polypeptide from protease and peptidase degradation within the blood of the subject. Accordingly, in some aspects, a polypeptide useful for the present disclosure can comprise both D-amino acids and L-amino acids. For instance, in some aspects, a polypeptide described herein comprises a D-amino acid at the N-terminus and L-amino acid at all other amino acid residues. In some aspects, a polypeptide described herein comprises a D-amino acid at the C-terminus and L-amino acid at all other amino acid residues. In some aspects, a polypeptide described herein comprises a D-amino acid at both the N-terminus and the C-terminus, and L-amino acid at all other amino acid residues.

As described herein, in some aspects, a LRRC4 family mimic molecule described above comprises a polypeptide having an amino acid sequence set forth in any one of SEQ ID NOs: 29 (YTYFTTVTVE), 30 (YSFFTTVTVE), and 31 (FSYFSTVTVE), with one, two, three, four, five, or six amino acids different from the amino acid sequence (e.g., substitutions).

In some aspects, a LRRC4 family mimic molecule useful for the present disclosure comprises additional modifications at the N-terminus, C-terminus, or both the N-terminus and the C-terminus of the polypeptide, wherein the additional modifications can increase the stability of the polypeptide. For instance, in some aspects, the N-terminal end of the polypeptide has been methylated. Non-limiting examples of additional modifications that can be performed at the N-terminus and/or C-terminus include: Fmoc, PEGylation, acetylation, or combinations thereof. In some aspects, to increase stability, the polypeptide can be cyclized. Any suitable methods known in the art can be used to make such modifications.

As further described elsewhere in the present disclosure, in some aspects, a molecule useful for the present disclosure comprises a FAM19A5 binding domain of members of the LRRC4 family protein and an additional moiety, which is capable of improving one or more properties of the molecules (e.g., binding affinity of the molecules to the FAM19A5 protein). As demonstrated herein (see, e.g., Example 10), Applicant has identified that the addition of the juxta-membrane sequence of members of the LRRC4 protein family can greatly improve the binding affinity of the molecules to FAM19A5 protein. The juxta-membrane sequence is highly conserved among members of the LRRC4 family and set forth in SEQ ID NO: 151 (LDEVMKTTK)(LRRC4 and LRRC4B) and SEQ ID NO: 152 (IDEVMKTTK) (LRRC4C) (see also FIG. 22D).

Accordingly, in some aspects, a molecule described herein comprises the FAM19A5 binding domain of the LRRC4 protein (i.e., YSFFTTVTVE; SEQ ID NO: 30) and the juxta-membrane sequence set forth in SEQ ID NO: 151 (LDEVMKTTK). In some aspects, a molecule described herein comprises the FAM19A5 binding domain of the LRRC4 protein (i.e., YSFFTTVTVE; SEQ ID NO: 30) and the juxta-membrane sequence set forth in SEQ ID NO: 152 (IDEVMKTTK). In some aspects, a molecule described herein comprises the FAM19A5 binding domain of the LRRC4B protein (i.e., YTYFTTVTVE; SEQ ID NO: 29) and the juxta-membrane sequence set forth in SEQ ID NO: 151 (LDEVMKTTK). In some aspects, a molecule described herein comprises the FAM19A5 binding domain of the LRRC4 protein (i.e., YTYFTTVTVE; SEQ ID NO: 29) and the juxta-membrane sequence set forth in SEQ ID NO: 152 (IDEVMKTTK). In some aspects, a molecule described herein comprises the FAM19A5 binding domain of the LRRC4B protein (i.e., FSYFSTVTVE; SEQ ID NO: 31) and the juxta-membrane sequence set forth in SEQ ID NO: 151 (LDEVMKTTK). In some aspects, a molecule described herein comprises the FAM19A5 binding domain of the LRRC4 protein (i.e., FSYFSTVTVE; SEQ ID NO: 31) and the juxta-membrane sequence set forth in SEQ ID NO: 152 (IDEVMKTTK). In some aspects, the juxta-membrane is added to the C-terminus of the molecule.

As is apparent from the present disclosure, any of the modifications described herein to improve one or more properties of a molecule (e.g., amino acid substitutions, addition of a juxta-membrane sequence, D-amino acids) can be used in combination. For instance, in some aspects, a molecule useful for the present disclosure (e.g., polypeptide) comprises: (i) the amino acid sequence set forth in SEQ ID NO: 18 (GYTYFTTVTVETLETQPGEE) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 18 (GYTYFTTVTVETLETQPGEE) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 17 (GYTYFTTVTVETLETQ) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 17 (GYTYFTTVTVETLETQ) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 19 (GYTYFTTVTVETLETQPGEKEPPGPTTD) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 19 (GYTYFTTVTVETLETQPGEKEPPGPTTD) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 143 (GYTYFTTVTVETLETQPGEEA) with amino acid modifications at residues T12 and L13; and (ii) a juxta-membrane sequence (e.g., SEQ ID NO: 151 or SEQ ID NO: 152) at the C-terminus of the molecule. In some aspects, a molecule useful for the present disclosure (e.g., polypeptide) comprises: (i) the amino acid sequence set forth in SEQ ID NO: 143 (GYTYFTTVTVETLETQPGEEA) with amino acid modifications at residues T12 and L13; (ii) D-amino acids at the N-terminus and/or C-terminus; and (iii) a juxta-membrane sequence (e.g., SEQ ID NO: 151 or SEQ ID NO: 152) at the C-terminus of the molecule.

In some aspects, a LRRC4 family mimic molecule described herein can comprise one or more additional peptides that allow the molecule to be specifically targeted to different tissues, e.g., when administered to a subject. For instance, in some aspects, a LRRC4 family mimic molecule comprises a peptide that allows the molecule to penetrate across the blood-brain barrier (also referred to herein as “BBB shuttles”). Examples of such BBB shuttles are known in the art. Non-limiting examples are provided in Table 5 (below). See, e.g., Oller-Salvia et al., Chem Soc Rev 45:4690 (2016).

TABLE 5
BBB Shuttles

| SEQ ID | | |
|---|---|---|
| NO | Peptide | Sequence |
| 32 | Angiopep-2 | TFFYGGSRGKRNNFKTEEY-OH |
| 33 | ApoB (3371-3409) | SSVIDALQYKLEGTTRLTRKRGLKLATALSLSNKFVEGS |
| 34 | ApoE (159-167)₂ | (LRKLRKRLL)₂ |
| 35 | Peptide-22 | Ac-C(&)MPRLRGC(&)-N_{H2} |
| 36 | THR | THRPPMWSPVWP-NH₂ |
| 37 | THR retro-enantio | pwvpswmpprht-NH₂ |
| 38 | CRT | C(&)RTIGPSVC(&) |
| 39 | Leptin30 | YQQILTSMPSRNVIQISNDLENLRDLLHVL |
| 40 | RVG29 | YTIWMPENPRPGTPCDIFTNSRGKRASNG-OH |
| 41 | CDX | GreirtGraerwsekf-OH |
| 42 | Apamin | C(&1)NC(&₂)KAPETALC(&₁)-ARRC(&₂)QQH-NH₂ |
| 43 | Mini Ap-4 | [Dap](&)KAPETALD(&) |
| 44 | GSH | γ-L-glutamyl-CG-OH |
| 45 | G23 | HLNILSTLWKYRC |
| 46 | g7 | GFtGFLS(O-b-Glc)-NH₂ |
| 47 | TGN | TGNYKALHPHNG |
| 48 | TAT (47-57) | YGRKKRRQRRR-NH₂ |
| 49 | SynB1 | RGGRLSYSRRRFSTSTGR |
| 50 | Diketopiperazines | &(N-MePhe)-(N-MePhe)Diketopiperazines |
| 51 | PhPro | (Phenylproline)4-NH₂ |

Nomenclature for cyclic peptides (&) is adapted to the 3-letter amino acid code from the one described in Spengler et al., J Pept Res 65: 550-555 (2005); [Dap] stands for diaminopropionic acid.

In some aspects, a LRRC4 family mimic molecule useful for the present disclosure comprises a fusion protein. For instance, in some aspects, a LRRC4 family mimic molecule described herein can comprise: (i) any of the polypeptides of the present disclosure, and (ii) a half-life extending moiety. Any suitable half-life extending moieties known in the art can be used to generate the fusion proteins of the present disclosure. Non-limiting examples of such half-life extending moieties include: a Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, a LRRC4 family mimic molecule comprises a protein-drug conjugate. For instance, in some aspects, the polypeptide of the LRRC4 family mimic can be conjugated to a therapeutic agent, such as those that are useful for treating a disease or disorder.

The protein-drug conjugates described herein can be prepared by methods known in the art. In some aspects, conjugation methods result in linkages which are substantially (or nearly) non-immunogenic, e.g., peptide- (i.e., amide-), sulfide-, (sterically hindered), disulfide-, hydrazone-, and ether linkages. These linkages are nearly non-immunogenic and show reasonable stability within serum (see, e.g., Senter, P. D., Curr. Opin. Chem. Biol. 13 (2009) 235-244; WO 2009/059278; WO 95/17886, each of which is incorporated herein by reference in its entirety).

Depending on the biochemical nature of the moiety and the polypeptides, different conjugation strategies can be employed (see, e.g., Hackenberger, C. P. R., and Schwarzer, D., Angew. Chem. Int. Ed. Engl. 47 (2008) 10030-10074). In some aspects, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al., Chem. Biol. 15 (2008) 128-136; and Protease-catalyzed formation of C— N bonds is used by Bordusa, F., Highlights in Bioorganic Chemistry (2004) 389-403).

Site specific reaction and covalent coupling can also be achieved by the selective reaction of terminal amino acids with appropriate modifying reagents. The reactivity of an N-terminal cysteine with benzonitrils (see Ren, H. et al., Angew. Chem. Int. Ed. Engl. 48 (2009) 9658-9662) can be used to achieve a site-specific covalent coupling. Native chemical ligation can also rely on C-terminal cysteine residues (Taylor, E. Vogel; Imperiali, B, Nucleic Acids and Molecular Biology (2009), 22 (Protein Engineering), 65-96).

The moiety can also be a synthetic peptide or peptide mimic. In such cases, a polypeptide can be chemically synthesized, amino acids with orthogonal chemical reactivity can be incorporated during such synthesis (see, e.g., de Graaf, A. J. et al., Bioconjug. Chem. 20 (2009) 1281-1295). To obtain a mono-labeled polypeptide, the conjugate with 1:1 stoichiometry can be separated by chromatography from other conjugation side-products. This procedure can be facilitated using a dye labeled binding pair member and a charged linker. With this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

### IV. Pharmaceutical Compositions

Provided herein are compositions comprising a therapeutic agent (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist) having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a pharmaceutical composition useful for the present disclosure comprises any of the therapeutic agents described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist), and optionally one or more additional prophylactic or therapeutic agents, in a pharmaceutically acceptable carrier. In some aspects, pharmaceutical compositions comprise any of the therapeutic agents described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist), and optionally one or more additional prophylactic of therapeutic agents, in a pharmaceutically acceptable carrier. In some aspects, the therapeutic agents described herein are the only active ingredient included in the pharmaceutical composition

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition can be formulated for any route of administration to a subject. Specific examples of routes of administration include intranasal, oral, parenterally, intrathecally, intra-cerebroventricularly, pulmonarily, subcutaneously, or intraventricularly. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of a therapeutic agent described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist) include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising a therapeutic agent are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A pharmaceutical composition can be formulated as an aerosol for topical application, such as by inhalation (see, e.g., U.S. Pat. Nos. 4,044,126, 4,414,209 and 4,364,923). These formulations for administration to the respiratory tract can be in the form of an aerosol or solution for a nebulizer, or as a microfine powder for insufflations, alone or in combination with an inert carrier such as lactose. In such a case, the particles of the formulation can, in some aspects, have diameters of less than about 50 microns, e.g., less than about 10 microns.

A pharmaceutical composition can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer any of the therapeutic agents described herein. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

In some aspects, a pharmaceutical composition described herein is a lyophilized powder, which can be reconstituted for administration as solutions, emulsions and other mixtures. It can also be reconstituted and formulated as solids or gels. The lyophilized powder is prepared by dissolving any of the therapeutic agents described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium, or potassium phosphate or other such buffer known to those of skill in the art. In some aspects, the buffer is at about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of any of the therapeutic agents described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist). The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

In some aspects, a pharmaceutical composition comprising any of the therapeutic agents described herein can also be formulated to be targeted to a particular tissue, receptor, or other area of the body of the subject to be treated. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542, and 5,709,874.

The compositions to be used for in vivo administration can be sterile. In some aspects, this can be accomplished by filtration through, e.g., sterile filtration membranes.

### V. Nucleic Acids, Vectors, Host Cells

Further aspect described herein pertains to one or more nucleic acid molecules (also referred to herein as “nucleic acids” or derivatives thereof) that encode a therapeutic agent (e.g., a LRRC4 family mimic molecule and/or FAM19A5 antagonist described herein). The nucleic acids can be present in whole cells, in a cell lysate, or in a partially purified or substantially pure form. In some aspects, the nucleic acid is a DNA sequence and/or an RNA sequence (e.g., mRNA). In some aspects, the nucleic acids comprise a modified nucleotide analog. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. In some aspects, a nucleic acid molecule can or cannot contain intronic sequences. In some aspects, the nucleic acid is a cDNA molecule. Nucleic acids described herein can be obtained using standard molecular biology techniques known in the art.

In some aspects, the present disclosure provides a vector comprising an isolated nucleic acid molecule encoding a therapeutic agent disclosed herein (e.g., a LRRC4 family mimic molecule and/or FAM19A5 antagonist described herein). Suitable vectors for the disclosure include, but are not limited to, expression vectors, viral vectors, and plasmid vectors. In some aspects, the vector is a viral vector.

As used herein, an “expression vector” refers to any nucleic acid construct which contains the necessary elements for the transcription and translation of an inserted coding sequence, or in the case of a RNA viral vector, the necessary elements for replication and translation, when introduced into an appropriate host cell. Expression vectors can include plasmids, phagemids, viruses, and derivatives thereof.

As used herein, “viral vectors” include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Additionally, plasmids can be custom designed using standard molecular biology techniques to remove and/or add specific fragments of DNA.

Also encompassed herein is a method for making a therapeutic agent disclosed herein (e.g., a LRRC4 family mimic molecule and/or FAM19A5 antagonist described herein). In some aspects, such a method can comprise expressing the molecule (e.g., a LRRC4 family mimic molecule and/or FAM19A5 antagonist described herein) in a cell comprising a nucleic acid molecule encoding the molecule. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### VI. Kits

Also provided herein are kits comprising one or more of the therapeutic agents described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist). As is apparent from the present disclosure, in some aspects, a kit comprising a LRRC4 family mimic molecule can be useful in diagnosing a disease or disorder associated with increased FAM19A5 protein expression in a subject. In some aspects, provided herein is a pharmaceutical pack or kit comprising one or more containers filled with one or more of the ingredients of the pharmaceutical compositions described herein, optional an instruction for use. In some aspects, the kits contain a pharmaceutical composition described herein and any prophylactic or therapeutic agent, such as those described herein.

The following examples are offered by way of illustration and not by way of limitation.

### EXAMPLES

### Example 1: Construction of a LRRC4 Family Mimic Molecule-Based ELISA Assay

To assess the ability of the LRRC4 mimic molecules described herein to measure the amount of FAM19A5 protein in a sample, sandwich ELISA assays were constructed as shown in FIGS. 1A-IC. Briefly, a 96-well plate was coated with one of the following: (i) a monoclonal anti-FAM19A5 antibody (FIG. 1A); (ii) a Fc-conjugated LRRC4B peptide fragment (FIG. 1B); and (iii) the LRRC4B peptide fragment that is not conjugated to Fc but instead conjugated to a His tag Tobacco Etch Virus (TEV) protease sequence (“His-TEV-LRRC4B”). Then, as described further below, biological samples (e.g., blood or cerebrospinal fluid) obtained from a subject were added to the wells of the plate and subsequently, incubated. After the incubation, a HRP-conjugated monoclonal anti-FAM19A5 antibody was added to the relevant wells, and UV absorbance was used to quantify the amount of FAM19A5 protein. While the present example uses LRRC4B peptide fragment, it is apparent from the present disclosure that any of the LRRC4 mimic molecules described herein can be used (e.g., LRRC4 peptide fragment and/or LRRC4C peptide fragment).

### Example 2: Comparison of Basal FAM19A5 Protein Levels in Mice, Rat, Monkeys, and Human

Based on assays available in the art, FAM19A5 protein levels in the blood of both humans and mice have been determined (about 100 pg/mL and about 400 ng/mL, respectively). See Lee et al., Diab Vasc Dis Res 16(6): 530-538 (November 2019); and Wang et al., Circulation 138(1): 48-63 (July 2018), each of which is incorporated herein by reference in its entirety. However, the accuracy of such assays is questionable as both of the anti-FAM19A5 ELISA kits used in Lee et al. (from Mybiosource) and Wang et al. (from R&D Systems) failed to detect recombinant FAM19A5 protein produced in mammalian cells. Like many of the assays available in the art, those assays used anti-FAM19A5 antibodies which were produced using FAM19A5 protein purified from E coli as the immunogen, suggesting that such a FAM19A5 protein may fold differently from not produced in mammalian cells. Therefore, to compare the methods described herein to the methods available in the art, the His-TEV-LRRC4B-based ELISA assay described in Example 1 was used to quantify the level of FAM19A5 protein in the peripheral blood of naïve mice (both wild-type and FAM19A5 knock-out), rats, monkeys (both cynomolgus and marmoset), and humans. Human blood samples were purchased from BioIVT (Westbury, NY).

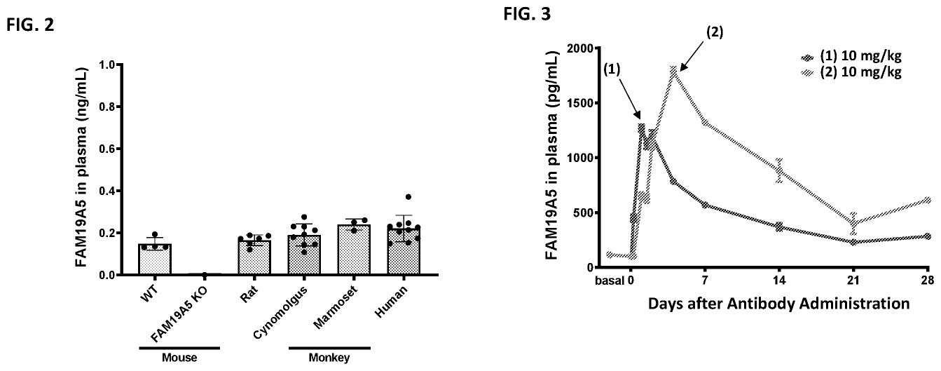

As shown in FIG. 2, FAM19A5 protein levels in the blood of naïve mice (wild-type), rats, and monkeys were estimated to be about 100-200 pg/mL. In humans, the FAM19A5 protein level was determined to be about 150-400 pg/mL. To confirm the specificity of the assay, no detectable amounts of FAM19A5 protein were observed in FAM19A5 knockout animals.

The above results demonstrate that contrary to the published results described above, the basal level of FAM19A5 protein present in the peripheral blood of various mammalian sources is generally comparable, including mice and humans.

### Example 3: Analysis of FAM19A5 Protein Level after FAM19A5 Antagonist Administration

To further assess the ability of LRRC4 family mimic molecules described herein to detect FAM19A5 protein present in a sample, a single intravenous administration of an anti-FAM19A5 antibody (at either 10 mg/kg or 50 mg/kg) was administered to naïve rats. Then, at various time points post-antibody administration, blood and cerebrospinal fluid (CSF) were collected from the animals and the level of FAM19A5 protein present was assessed using the His-TEV-LRRC4B-based ELISA assay described in Example 1.

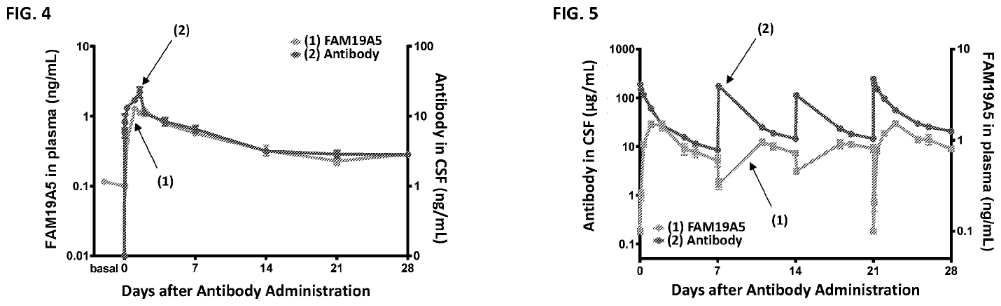

As shown in FIG. 3, administration of the anti-FAM19A5 antibody resulted in a rapid increase (within 6 hours of administration) in blood FAM19A5 protein levels in a dose and time-dependent manner. While the increase in blood FAM19A5 protein level was slightly delayed in animals treated with the higher dose of the anti-FAM19A5 antibody, it should be noted that the FAM19A5 protein concentrations observed shortly after antibody administration does not likely reflect the actual FAM19A5 concentration. As described herein, it was observed that high concentration of the administered anti-FAM19A5 antibody present in the same could interfere with the accuracy of the FAM19A5 ELISA. However, despite such slight delay, compared to animals treated with the lower dose of the anti-FAM19A5 antibody, rats that received the higher dose generally had greater amount of FAM19A5 protein present in the blood at majority of the time points assessed. Similar trend was observed in the CSF of the animals (see FIG. 4).

Considering that 1) the anti-FAM19A5 antibody concentration in the CSF partly reflected the concentration of the antibody passing through the brain parenchyma, 2) the increase in anti-FAM19A5 antibody concentration preceded the increase in FAM19A5 protein levels in the blood, and 3) the anti-FAM19A5 antibody concentration in the CSF and the FAM19A5 protein concentration in the blood had approximately 1:1 molar ratio at about 4 days after antibody administration, the above results suggested that the increased blood FAM19A5 protein level was likely related to the anti-FAM19A5 antibody passing through the brain parenchyma.

Next, the His-TEV-LRRC4B-based ELISA assay described in Example 1 was used to assess blood FAM19A5 protein level after weekly administration of an anti-FAM19A5 antibody. Briefly, naïve rats received an intravenous administration of the anti-FAM19A5 antibody weekly for four weeks (10 mg/kg per dose). Blood was collected at various time points during the course of the antibody administrations, and the blood FAM19A5 protein level was assessed. The concentration of the anti-FAM19A5 antibody in the blood was also assessed using an ELISA method that uses rabbit anti-human IgG Fc and HRP-conjugated goat anti-human IgG kappa light chain antibodies.

As shown in FIG. 5, after each dose, peak blood FAM19A5 protein level occurred at around day 3 post administration and gradually decreased by day 7 post administration. FAM19A5 protein levels at 2 hours post antibody administration were much lower than those before the administration, suggesting that high antibody concentration present in a sample can again interfere with FAM19A5 ELISA. These results suggest that blood FAM19A5 protein level can be a pharmacodynamic (PD) marker for the administered anti-FAM19A5 antibody.

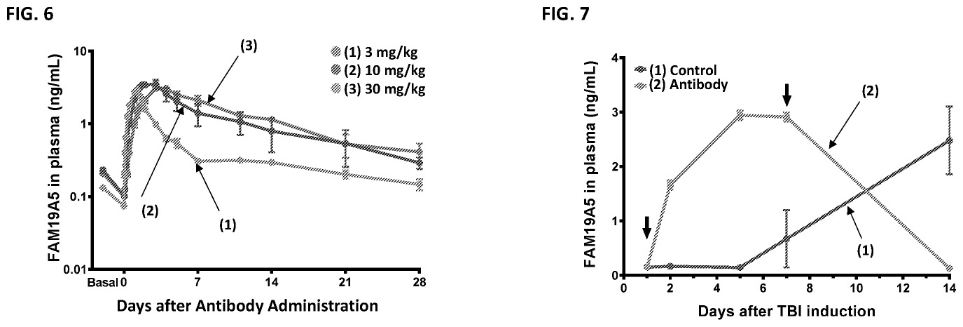

As confirmation, cynomolgus monkeys were treated with a single intravenous administration of the anti-FAM19A5 antibody (at 3 mg/kg, 10 mg/kg, or 30 mg/kg). Blood was collected at various time points post antibody administration, and the concentration of the FAM19A5 protein was assessed using the His-TEV-LRRC4B-based ELISA assay described in Example 1. As shown in FIG. 6, similar to that observed in the rat samples, higher dose of the anti-FAM19A5 antibody resulted in greater blood FAM19A5 protein level. Additionally, blood FAM19A5 protein level was generally maintained in the different treatment groups until about 21 days after antibody administration.

The above result confirmed that blood FAM19A5 protein level can be a suitable proximal PD marker for anti-FAM19A5 antibody action and provided evidence for target engagement of the administered anti-FAM19A5 antibody.

### Example 4: Analysis of FAM19A5 Protein Expression in Traumatic Brain Injury-Induced Mice

To assess the diagnostic capability of the LRRC4 family mimic molecules described herein, a traumatic brain injury (TBI) mouse model was used. Briefly, six-month-old female mice were deeply anesthetized with sodium pentobarbital (50 mg/kg). Cryogenic TBI was performed by placing a prechilled iron rod on the calvarium for 1 min. The mouse's skin was then sutured and housed in the same manner as normal mice. Moon et al., Neuro Report 22: 304-308 (2011). Approximately, one day after TBI induction, the animals were treated with anti-FAM19A5 antibody. As control, non-treated TBI-induced mice and TBI-induced mice treated with human IgG antibody were used. Blood was collected from the animals at various time points, and the concentration of the FAM19A5 protein in the peripheral blood was assessed using the LRRC4 family mimic molecule-based ELISA assay described in Example 1.

As shown in FIG. 7, starting at about day 5 post TBI induction, a significant increase in FAM19A5 protein expression was observed in the peripheral blood of the control animals. And, in animals treated with the anti-FAM19A5 antibody, there was a sharp increase in FAM19A5 proteins levels in the peripheral blood until about day 7 post TBI induction. Afterwards, the FAM19A5 protein levels gradually declined, and by day 14 post TBI induction, FAM19A5 protein levels returned to baseline (i.e., levels prior to administration of the anti-FAM19A5 antibody).

The above results confirm that the LRRC4 family mimic molecules described herein can be useful as a diagnostic tool. Additionally, the results suggest that anti-FAM19A5 antibody administration can accelerate FAM19A5 secretion into the blood from damaged brain. Therefore, it is plausible that anti-FAM19A5 administration can induce higher secretion of FAM19A5 protein in patients with brain pathology than in normal individuals.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

What is claimed is:

1. A method of determining an expression level of a family with sequence similarity 19, member A5 (“FAM19A5”) protein in a subject in need thereof, comprising: (i) contacting a biological sample of the subject with a Leucine Rich Containing 4 (“LRRC4”) family mimic molecule, which is capable of specifically binding to the FAM19A5 protein, and (ii) measuring the expression level of the FAM19A5 protein (“FAM19A5 protein expression level”) in the biological sample.

2. A method of diagnosing a disease or disorder in a subject in need thereof, comprising: (i) contacting a biological sample of the subject with a Leucine Rich Containing 4 (“LRRC4”) family mimic molecule, which is capable of specifically binding to a family with sequence similarity 19, member A5 (“FAM19A5”) protein, and (ii) measuring an expression level of the FAM19A5 protein (“FAM19A5 protein expression level”) in the biological sample.

3. The method of claim 2, wherein the disease or disorder is associated with an increased FAM19A5 protein expression level.

4. The method of any one of claims 1 to 3, wherein the FAM19A5 protein expression level in the biological sample is increased compared to a reference (e.g., corresponding expression level in a subject who is not afflicted with the disease or disorder).

5. The method of claim 4, wherein the FAM19A5 protein expression level is increased by about 1-fold, by about 2-fold, by about 3-fold, by about 4-fold, by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, or by about 50-fold compared to the reference.

6. A method of identifying a subject who is suitable for a treatment with an antagonist against a family with sequence similarity 19, member A5 (“FAM19A5”) protein (“FAM19A5 antagonist”), comprising: (i) contacting a biological sample of the subject with a Leucine Rich Containing 4 (“LRRC4”) family mimic molecule, which is capable of specifically binding to the FAM19A5 protein, and (ii) measuring an expression level of the FAM19A5 protein (“FAM19A5 protein expression level”) in the biological sample, wherein the subject is suitable for the treatment if the FAM19A5 protein expression level is increased compared to a reference (e.g., corresponding expression level in a subject who is not suffering from the disease or disorder).

7. The method of claim 6, wherein the subject is afflicted with a disease or disorder which is associated with an increased FAM19A5 protein expression level.

8. The method of claim 6 or 7, wherein the FAM19A5 protein expression level is increased by about 1-fold, by about 2-fold, by about 3-fold, by about 4-fold, by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, or by about 50-fold compared to the reference.

9. A method of identifying a subject responsive to a treatment with an antagonist against a family with sequence similarity 19, member A5 (“FAM19A5”) protein (“FAM19A5 antagonist”), comprising (i) contacting a biological sample of the subject with a Leucine Rich Containing 4 (“LRRC4”) family mimic molecule, which is capable of specifically binding to the FAM19A5 protein, and (ii) measuring the expression level of the FAM19A5 protein (“FAM19A5 protein expression level”) in the biological sample, wherein the subject is responsive to the treatment if the FAM19A5 protein expression level is decreased compared to a reference (e.g., corresponding expression level in the subject prior to the treatment).

10. The method of claim 9, wherein the subject had previously received an administration of the FAM19A5 antagonist.

11. The method of claim 9 or 10, wherein the FAM19A5 protein expression level is decreased by at least about 5%, by at least about 10%, by at least about 20%, by at least about 30%, by at least about 40%, by at least about 50%, by at least about 60%, by at least about 70%, by at least about 80%, by at least about 90%, or by about 100% compared to the reference.

12. The method of any one of claims 9 to 11, which further comprises administering to the subject an additional dose of the FAM19A5 antagonist after the contacting and measuring.

13. A method of treating a disease or disorder associated with an increased expression level of a family with sequence similarity 19, member A5 (“FAM19A5”) protein in a subject in need thereof, comprising administering to the subject a treatment for the disease or disorder and measuring an expression level of the FAM19A5 protein (“FAM19A5 protein expression level”) by contacting a biological sample of the subject with a Leucine Rich Containing 4 (“LRRC4”) family mimic molecule, which is capable of specifically binding to the FAM19A5 protein.

14. The method of claim 13, which further comprises administering to the subject an additional dose of the treatment if the FAM19A5 protein expression level is increased compared to a reference (e.g., corresponding expression level in a subject who is not afflicted with the disease or disorder).

15. The method of claim 14, wherein the FAM19A5 protein expression level is increased by about 1-fold, by about 2-fold, by about 3-fold, by about 4-fold, by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, or by about 50-fold compared to the reference.

16. The method of any one of claims 1 to 15, wherein the FAM19A5 protein expression level measured by an enzyme linked immunosorbent assay (ELISA), immunohistochemistry, Western blotting, radioimmunoassay, radical-immunodiffusion, immunoprecipitation assay, Ouchterlony immunodiffusion method, rocket immunoelectrophoresis, tissue immunostaining method, complement fixation assay, FACS, protein chip, SIMOA technology, or any combination thereof.

17. The method of any one of claims 1 to 16, wherein the biological sample comprises a blood, cerebral spinal fluid (CSF), serum, plasma, tissue, cell culture media, saliva, urine, or any combination thereof.

18. The method of any one of claims 1 to 17, wherein the contacting and the measuring is performed in vitro.

19. The method of any one of claims 13 to 18, wherein the treatment comprises an antagonist against the FAM19A5 (“FAM19A5 antagonist), a Leucine Rich Containing 4 (“LRRC4”) family mimic molecule, or both.

20. The method of any one of claims 6 to 12 and 19, wherein the FAM19A5 antagonist comprises an antibody or an antigen-binding portion thereof, antisense oligonucleotide, siRNA, shRNA, miRNA, dsRNA targeting FAM19A5, aptamer, PNA, vector including the same, or any combinations thereof.

21. The method of claim 20, wherein the FAM19A5 antagonist is an antibody or an antigen-binding portion thereof.

22. The method of any one of claims 1 to 20, wherein the LRRC4 family mimic molecule is not an antibody or an antigen-binding portion thereof.

23. The method of any one of claims 1 to 22, wherein the LRRC4 family mimic molecule comprises a polypeptide comprising, consisting of, or consisting essentially of a domain of a LRRC4 protein family member, wherein the domain is capable of binding to a FAM19A5 protein (“FAM19A5 binding domain”), and wherein the polypeptide is shorter than the corresponding full-length LRRC4 protein family member (SEQ ID NO: 4; SEQ ID NO: 5; or SEQ ID NO: 6).

24. The method of claim 23, wherein the FAM19A5 binding domain is about 10 to about 23 amino acids in length.

25. The method of claim 24, wherein the FAM19A5 binding domain is about 10, about 11, about 12, about 13, about 14, about 15, about 16, about 17, about 18, about 19, about 20, about 21, about 22, or about 23 amino acids in length.

26. The method of claim 25, wherein the FAM19A5 binding domain is about 10 amino acids in length.

27. The method of any one of claims 1 to 26, wherein the LRRC4 family mimic molecule comprises a polypeptide comprising an amino acid sequence having the following formula (from N-terminus to C-terminus):
A-(T/S)-B, wherein: (Formula I)
(i) A comprises X1-(T/S)-(Y/F)—F-X5; wherein:
X1 is tyrosine (Y), phenylalanine (F), valine (V), leucine (L), or isoleucine (I);
(T/S) is threonine (T) or serine (S);
(Y/F) is tyrosine (Y) or Phenylalanine (F); and
X5 is any amino acid; and
(ii) B comprises (V/I)-T-V-(E/V); wherein:
(V/I) is valine (V) or isoleucine (I); and
(E/V) is glutamic acid (E) or valine (V).

28. The method of any one of claims 1 to 26, wherein the LRRC4 family mimic molecule comprises a polypeptide comprising an amino acid sequence having the following formula (from N-terminus to C-terminus):
A-(T/S)-B, wherein: (Formula I)
(i) A comprises (Y/W/M)-(T/Y)-(Y/W)-(F/Y/W)-(T/Y); wherein:
(Y/W/M) is tyrosine (Y), tryptophan (W), or methionine (M);
(T/Y) is threonine (T) or tyrosine (Y);
(Y/W) is tyrosine (Y) or tryptophan (W); and
(F/Y/W) is phenylalanine (F), tyrosine (Y), or tryptophan (W); and
(ii) B comprises X7-(T/S/Y)-X9-X10; wherein:
X7 is valine (V), tyrosine (Y), phenylalanine (F), leucine (L), tryptophan (W), or methionine (M);
(T/S/Y) is threonine (T), serine (S), or tyrosine (Y);
X9 is valine (V), isoleucine (I), tyrosine (Y), phenylalanine (F), leucine (L), tryptophan (W), or methionine (M); and
X10 is glutamic acid (E), aspartic acid (D), isoleucine (I), tyrosine (Y), phenylalanine (F), methionine (M), or tryptophan (W).

29. The method of any one of claims 1 to 26, wherein the LRRC4 family mimic molecule comprises a polypeptide comprising an amino sequence having the following formula (from N-terminus to C-terminus):
X1-X2-X3-F-X5-T-X7-T-V-X10, wherein: (Formula II)
X1 is Y, F, V, L, or I;
X2 is T or S;
X3 is Y or F;
X5 is any amino acid;
X7 is V or I; and/or
X10 is E or V.

30. The method of any one of claims 1 to 26, wherein the LRRC4 family mimic molecule comprises a polypeptide comprising an amino acid sequence having the following formula: (from N-terminus to C-terminus):
X1-X2-X3-X4-X5-X6-X7-X8-X9-X10, wherein: (Formula VI)
X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W.

31. The method of claim 30, wherein:
a. X1 is Y, F, V, L, or I;
b. X2 is T or S;
c. X3 is Y or F;
d. X4 is F;
e. X5 is T or S;
f. X6 is T;
g. X7 is V or I;
h. X8 is T;
i. X9 is V;
j. X10 is E or V; or
k. combinations thereof.

32. The method of any one of claims 1 to 31, wherein the LRRC4 family mimic molecule comprises a polypeptide comprising the amino acid sequence set forth in SEQ ID NO: 29 (YTYFTTVTVE).

33. The method of any one of claims 1 to 31, wherein the LRRC4 family mimic molecule comprises a polypeptide consisting of the amino acid sequence set forth in SEQ ID NO: 29 (YTYFTTVTVE).

34. The method of any one of claims 1 to 31, wherein the LRRC4 family mimic molecule comprises a polypeptide comprising the amino acid sequence set forth in SEQ ID NO: 20 (NYSFFTTVTVETTEISPEDTTRK).

35. The method of any one of claims 1 to 31, wherein the LRRC4 family mimic molecule comprises a polypeptide consisting of the amino acid sequence set forth in SEQ ID NO: 20 (NYSFFTTVTVETTEISPEDTTRK).

36. The method of any one of claims 1 to 31, wherein the LRRC4 family mimic molecule comprises a polypeptide comprising the amino acid sequence set forth in SEQ ID NO: 21 (NFSYFSTVTVETMEPSQDERTTR).

37. The method of any one of claims 1 to 31, wherein the LRRC4 family mimic molecule comprises a polypeptide consisting of the amino acid sequence set forth in SEQ ID NO: 21 (NFSYFSTVTVETMEPSQDERTTR).

38. The method of any one of claims 1 to 31, wherein the LRRC4 family mimic molecule comprises a polypeptide comprising the amino acid sequence set forth in SEQ ID NO: 18 (GYTYFTTVTVETLETQPGEE).

39. The method of any one of claims 1 to 31, wherein the LRRC4 family mimic molecule comprises a polypeptide consisting of the amino acid sequence set forth in SEQ ID NO: 18 (GYTYFTTVTVETLETQPGEE).

40. The method of any one of claims 29 to 39, wherein the amino acid at X2 is phosphorylated or O-glycosylated.

41. The method of any one of claims 1 to 31, wherein the LRRC4 family mimic molecule comprises a polypeptide, which comprises an amino acid sequence having at least about 60%, at least about 70%, at least about 80%, at least about 90%, at least about 95%, or at least about 99% sequence identity to the amino acid sequence set forth in SEQ ID NO: 29 (YTYFTTVTVE).

42. The method of any one of claims 1 to 31, wherein the LRRC4 family mimic molecule comprises a polypeptide, which comprises an amino acid sequence that is at least about 70%, at least about 75%, at least about 80%, at least about 85%, at least about 90%, at least about 95%, at least about 96%, at least about 97%, at least about 98%, or about 99% identical to the amino acid sequence set forth in SEQ ID NO: 5, SEQ ID NO: 4, or SEQ ID NO: 6 and contains at least one amino acid modification relative to the amino acid sequence set forth in SEQ ID NO: 5, SEQ ID NO: 4, or SEQ ID NO: 6, respectively.

43. The method of claim 42, wherein the at least one amino acid modification increases the binding of the polypeptide to the FAM19A5 protein.

44. The method of claim 42 or 43, wherein the at least one amino acid modification increases the stability of the LRRC4 family mimic molecule.

45. The method of any one of claims 42 to 44, wherein:
a. the amino acid residue at position 453 of SEQ ID NO: 5 is T or modified to S or Y;
b. the amino acid residue at position 454 of SEQ ID NO: 5 is T or modified to S or Y;
c. the amino acid residue at position 449 of SEQ ID NO: 5 is Y or modified to F, V, L, I, W, or M;
d. the amino acid residue at position 450 of SEQ ID NO: 5 is T or modified to S or Y;
e. the amino acid residue at position 451 of SEQ ID NO: 5 is Y or modified to F or W;
f. the amino acid residue at position 452 of SEQ ID NO: 5 is F or modified to Y or W;
g. the amino acid residue at position 455 of SEQ ID NO: 5 is V or modified to I, Y, F, L, W, or M;
h. the amino acid residue at position 456 of SEQ ID NO: 5 is T or modified to S or Y;
i. the amino acid residue at position 457 of SEQ ID NO: 5 is V or modified to I, Y, F, L, W, or M;
j. the amino acid residue at position 458 of SEQ ID NO: 5 is E or modified to D, V, I, Y, F, M, or W;
k. or combinations thereof.

46. The method of any one of claims 1 to 45, wherein the LRRC4 family mimic molecule does not comprise the transmembrane domain and/or the intracellular domain of a member of the LRRC4 protein family.

47. The method of any one of claims 1 to 46, wherein the LRRC4 family mimic molecule further comprises one or more additional amino acids at the N-terminus, the C-terminus, or both the N-terminus and the C-terminus of LRRC4 family mimic molecule.

48. The method of claim 47, wherein the one or more additional amino acids are hydrophilic amino acids, D-amino acids, or both.

49. The method of any one of claims 1 to 48, wherein the N-terminus, C-terminus, or both the N-terminus and the C-terminus of the LRRC4 family mimic molecule comprise a modification which increases the stability of the LRRC4 family mimic molecule.

50. The method of claim 49, wherein the modification comprises a Fmoc, PEGylation, acetylation, methylation, cyclization, or combinations thereof.

51. The method of any one of claims 1 to 50, wherein the LRRC4 family mimic molecule is a fusion protein.

52. The method of any one of claims 1 to 51, wherein the LRRC4 family mimic molecule further comprises a half-life extending moiety.

53. The method of claim 52, wherein the half-life extending moiety comprises a Fc, albumin, an albumin-binding polypeptide, Pro/Ala/Ser (PAS), a C-terminal peptide (CTP) of the 0 subunit of human chorionic gonadotropin, polyethylene glycol (PEG), long unstructured hydrophilic sequences of amino acids (XTEN), hydroxyethyl starch (HES), an albumin-binding small molecule, or a combination thereof.

54. The method of any one of claims 1 to 22, wherein the LRRC4 family mimic molecule comprises a small molecule.

55. The method of claim 54, wherein the LRRC4 family mimic molecule is a small molecule of Formula (I): 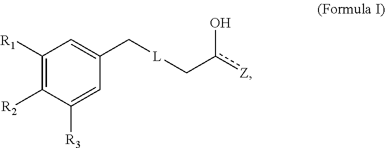
or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond.

56. The method of claim 55, wherein the LRRC4 family mimic molecule is selected from: 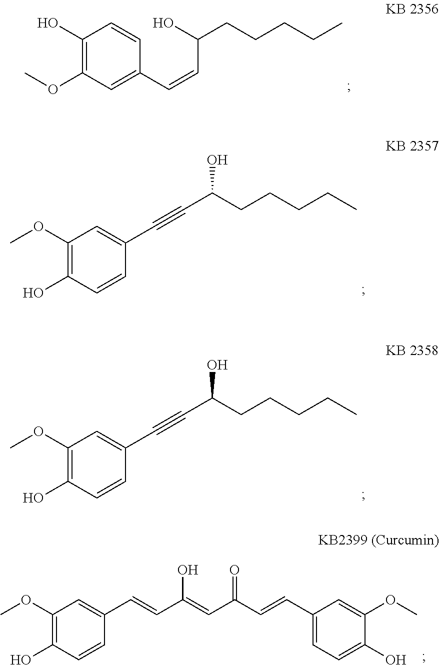
or a pharmaceutically acceptable salt thereof.

57. The method of any one of claims 1 to 22, wherein the LRRC4 family mimic molecule is a small molecule of Formula (II): 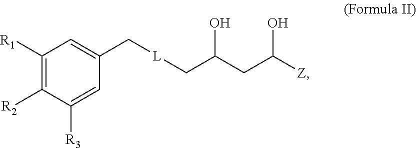
or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄) bicycloalkenyl, (7-14 membered) heterobicycloalkyl, (C₆-C₁₀) aryl, (5-10-membered) heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

58. The method of claim 57, wherein the LRRC4 family mimic molecule is selected from: 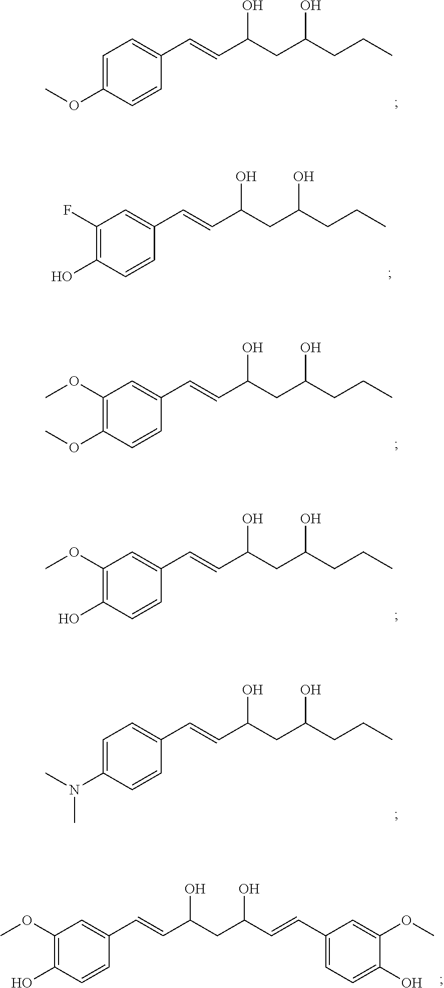
or a pharmaceutically acceptable salt thereof.

59. The method of any one of claims 1 to 22, wherein the LRRC4 family mimic molecule is a small molecule of Formula (III): 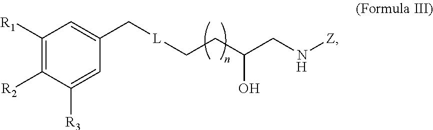
or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered) heterocycloalkyl, —Y—(C₇-C₁₄)bicycloalkyl, —Y—(C₇-C₁₄) bicycloalkenyl, —Y-(7-14 membered) heterobicycloalkyl, —Y—(C₆-C₁₀)aryl, and —Y-(5-10-membered) heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy,
(iii) L is single, double or triple bond, and
(iv) n is 0 or 1.

60. The method of claim 59, wherein the LRRC4 family mimic molecule is selected from: 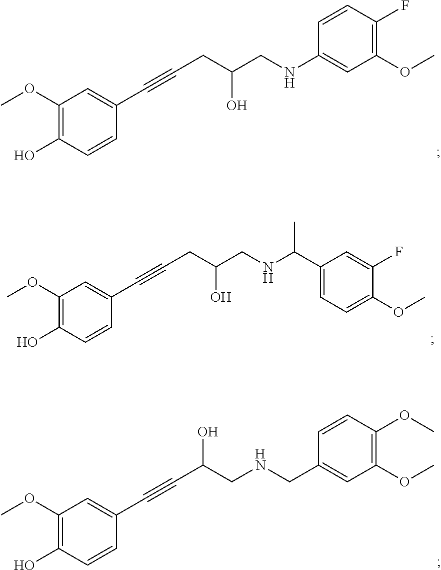
or a pharmaceutically acceptable salt thereof.

61. The method of any one of claims 1 to 60, wherein the LRRC4 family mimic molecule is capable of competing with members of the LRRC4 protein family for binding to the FAM19A5 protein.

62. The method of any one of claims 23 to 61, wherein the member of the LRRC4 protein family comprises a LRRC4 protein, LRRC4B protein, LRRC4C protein, or combinations thereof.

63. The method of any one of claims 1 to 4, 6 to 8, and 13 to 62, wherein the disease or disorder comprises an amyotrophic lateral sclerosis (ALS), Alzheimer's disease, glaucoma, diabetic retinopathy, neuropathic pain, spinal cord injury, traumatic brain injury, stroke, Parkinson's disease, or combinations thereof.


SEQUENCE LISTING (156 sequences)

SEQ ID NO: 1 - protein, 653 aa, synthetic construct
MKLLWQVTVHHHTWNAILLPFVYLTAQVWILCAAIAAAASAGPQNCPSVCSCSNQFSKVVCTRRGLSEVPQGIPSNTRYLNLMENNIQMIQADTFRHLHHLEVLQLGRNSIRQIEVGAFNGLASLNTLELFDNWLTVIPSGAFEYLSKLRELWLRNNPIESIPSYAFNRVPSLMRLDLGELKKLEYISEGAFEGLFNLKYLNLGMCNIKDMPNLTPLVGLEELEMSGNHFPEIRPGSFHGLSSLKKLWVMNSQVSLIERNAFDGLASLVELNLAHNNLSSLPHDLFTPLRYLVELHLHHNPWNCDCDILWLAWWLREYIPTNSTCCGRCHAPMHMRGRYLVEVDQASFQCSAPFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTKIIIGCFVAVTLLAAAMLIVFYKLRKRHQQRSTVTAARTVEIIQVDEDIPAATSAAATAAPSGVSGEGAVVLPTIHDHINYNTYKPAHGAHWTENSLGNSLHPTVTTISEPYIIQTHTKDKVQETQI

SEQ ID NO: 2 - protein, 713 aa, synthetic construct
MARARGSPCPPLPPGRMSWPHGALLFLWLFSPPLGAGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 3 - protein, 640 aa, synthetic construct
MLNKMTLHPQQIMIGPRFNRALFDPLLVVLLALQLLVVAGLVRAQTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTKIIIGCFVAITLMAAVMLVIFYKMRKQHHRQNHHAPTRTVEIINVDDEITGDTPMESHLPMPAIEHEHLNHYNSYKSPFNHTTTVNTINSIHSSVHEPLLIRMNSKDNVQETQI

SEQ ID NO: 4 - protein, 489 aa, synthetic construct
ASAGPQNCPSVCSCSNQFSKVVCTRRGLSEVPQGIPSNTRYLNLMENNIQMIQADTFRHLHHLEVLQLGRNSIRQIEVGAFNGLASLNTLELFDNWLTVIPSGAFEYLSKLRELWLRNNPIESIPSYAFNRVPSLMRLDLGELKKLEYISEGAFEGLFNLKYLNLGMCNIKDMPNLTPLVGLEELEMSGNHFPEIRPGSFHGLSSLKKLWVMNSQVSLIERNAFDGLASLVELNLAHNNLSSLPHDLFTPLRYLVELHLHHNPWNCDCDILWLAWWLREYIPTNSTCCGRCHAPMHMRGRYLVEVDQASFQCSAPFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTK

SEQ ID NO: 5 - protein, 541 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 6 - protein, 483 aa, synthetic construct
QTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTK

SEQ ID NO: 7 - protein, 124 aa, synthetic construct
VSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 8 - protein, 93 aa, synthetic construct
YTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 9 - protein, 95 aa, synthetic construct
GGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 10 - protein, 16 aa, synthetic construct
GGYTYFTTVTVETLET

SEQ ID NO: 11 - protein, 79 aa, synthetic construct
QPGEEALQPRGTEKEPPGPTTDGVWGGGRPGDAAGPASSSTTAPAPRSSRPTEKAFTVPITDVTENALKDLDDVMKTTK

SEQ ID NO: 12 - protein, 33 aa, synthetic construct
LNVSAVDPVAAGGTGSGGGGPGGSGGVGGGSGG

SEQ ID NO: 13 - protein, 34 aa, synthetic construct
LNVSAVDPVAAGGTGSGGGGPGGSGGVGGGSGGY

SEQ ID NO: 14 - protein, 39 aa, synthetic construct
YTYFTTVTVETLETQPGEEALQPRGTEKEPPGPTTDGVW

SEQ ID NO: 15 - protein, 13 aa, synthetic construct
YTYFTTVTVETLE

SEQ ID NO: 16 - protein, 124 aa, synthetic construct
VSAVDPVAAGGTGSGGGGPGGSGGVGGGSGGYTYFAAVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 17 - protein, 16 aa, synthetic construct
GYTYFTTVTVETLETQ

SEQ ID NO: 18 - protein, 20 aa, synthetic construct
GYTYFTTVTVETLETQPGEE

SEQ ID NO: 19 - protein, 28 aa, synthetic construct
GYTYFTTVTVETLETQPGEKEPPGPTTD

SEQ ID NO: 20 - protein, 23 aa, synthetic construct
NYSFFTTVTVETTEISPEDTTRK

SEQ ID NO: 21 - protein, 23 aa, synthetic construct
NFSYFSTVTVETMEPSQDERTTR

SEQ ID NO: 22 - protein, 132 aa, synthetic construct
MAPSPRTGSRQDATALPSMSSTFWAFMILASLLIAYCSQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 23 - protein, 125 aa, synthetic construct
MQLLKALWALAGAALCCFLVLVIHAQFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 24 - protein, 53 aa, synthetic construct
MYHHREWPARIIKTKQWCDMLPCLEGEGCDLLINRSGWTCTQPGGRIKTTTVS

SEQ ID NO: 25-26 - skipped (2 entries)

SEQ ID NO: 27 - protein, 10 aa, synthetic construct
XXXFXTXTVX

SEQ ID NO: 28 - skipped

SEQ ID NO: 29 - protein, 10 aa, synthetic construct
YTYFTTVTVE

SEQ ID NO: 30 - protein, 10 aa, synthetic construct
YSFFTTVTVE

SEQ ID NO: 31 - protein, 10 aa, synthetic construct
FSYFSTVTVE

SEQ ID NO: 32 - protein, 19 aa, synthetic construct
TFFYGGSRGKRNNFKTEEY

SEQ ID NO: 33 - protein, 39 aa, synthetic construct
SSVIDALQYKLEGTTRLTRKRGLKLATALSLSNKFVEGS

SEQ ID NO: 34 - protein, 18 aa, synthetic construct
LRKLRKRLLLRKLRKRLL

SEQ ID NO: 35 - protein, 8 aa, synthetic construct
CMPRLRGC

SEQ ID NO: 36 - protein, 12 aa, synthetic construct
THRPPMWSPVWP

SEQ ID NO: 37 - protein, 12 aa, synthetic construct
PWVPSWMPPRHT

SEQ ID NO: 38 - protein, 9 aa, synthetic construct
CRTIGPSVC

SEQ ID NO: 39 - protein, 30 aa, synthetic construct
YQQILTSMPSRNVIQISNDLENLRDLLHVL

SEQ ID NO: 40 - protein, 29 aa, synthetic construct
YTIWMPENPRPGTPCDIFTNSRGKRASNG

SEQ ID NO: 41 - protein, 16 aa, synthetic construct
GREIRTGRAERWSEKF

SEQ ID NO: 42 - protein, 18 aa, synthetic construct
CNCKAPETALCARRCQQH

SEQ ID NO: 43 - protein, 8 aa, synthetic construct
KAPETALD

SEQ ID NO: 44 - skipped

SEQ ID NO: 45 - protein, 13 aa, synthetic construct
HLNILSTLWKYRC

SEQ ID NO: 46 - protein, 7 aa, synthetic construct
GFTGFLS

SEQ ID NO: 47 - protein, 12 aa, synthetic construct
TGNYKALHPHNG

SEQ ID NO: 48 - protein, 11 aa, synthetic construct
YGRKKRRQRRR

SEQ ID NO: 49 - protein, 18 aa, synthetic construct
RGGRLSYSRRRFSTSTGR

SEQ ID NO: 50 - skipped

SEQ ID NO: 51 - protein, 4 aa, synthetic construct
PPPP

SEQ ID NO: 52 - protein, 678 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 53 - protein, 557 aa, synthetic construct
LSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 54 - protein, 484 aa, synthetic construct
RLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 55 - protein, 350 aa, synthetic construct
PVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 56 - protein, 261 aa, synthetic construct
VSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 57 - protein, 137 aa, synthetic construct
IIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 58 - protein, 541 aa, synthetic construct; identical to SEQ ID NO: 5

SEQ ID NO: 59 - protein, 328 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYA

SEQ ID NO: 60 - protein, 465 aa, synthetic construct
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 61 - protein, 213 aa, synthetic construct
PVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 62-64 - skipped (3 entries)

SEQ ID NO: 65 - protein, 14 aa, synthetic construct
YTYFTTVTVETLET

SEQ ID NO: 66 - protein, 173 aa, synthetic construct
VIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTK

SEQ ID NO: 67 - protein, 175 aa, synthetic construct
PFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTK

SEQ ID NO: 68 - protein, 18 aa, synthetic construct
KNYTYFTTVTVETNKETQ

SEQ ID NO: 69 - protein, 17 aa, synthetic construct
KNYTYFTTVTVENKETQ

SEQ ID NO: 70 - protein, 16 aa, synthetic construct
KNYTYFTTVTVNKETQ

SEQ ID NO: 71 - protein, 15 aa, synthetic construct
KNYTYFTTVTNKETQ

SEQ ID NO: 72 - protein, 14 aa, synthetic construct
KNYTYFTTVNKETQ

SEQ ID NO: 73 - protein, 13 aa, synthetic construct
KNYTYFTTNKETQ

SEQ ID NO: 74 - protein, 17 aa, synthetic construct
KNTYFTTVTVETNKETQ

SEQ ID NO: 75 - protein, 16 aa, synthetic construct
KNYFTTVTVETNKETQ

SEQ ID NO: 76 - protein, 15 aa, synthetic construct
KNFTTVTVETNKETQ

SEQ ID NO: 77 - protein, 14 aa, synthetic construct
KNTTVTVETNKETQ

SEQ ID NO: 78 - protein, 20 aa, synthetic construct
GATYFTTVTVETLETQPGEE

SEQ ID NO: 79 - protein, 20 aa, synthetic construct
GYAYFTTVTVETLETQPGEE

SEQ ID NO: 80 - protein, 20 aa, synthetic construct
GYTAFTTVTVETLETQPGEE

SEQ ID NO: 81 - protein, 20 aa, synthetic construct
GYTYATTVTVETLETQPGEE

SEQ ID NO: 82 - protein, 20 aa, synthetic construct
GYTYFATVTVETLETQPGEE

SEQ ID NO: 83 - protein, 20 aa, synthetic construct
GYTYFTAVTVETLETQPGEE

SEQ ID NO: 84 - protein, 20 aa, synthetic construct
GYTYFTTATVETLETQPGEE

SEQ ID NO: 85 - protein, 20 aa, synthetic construct
GYTYFTTVAVETLETQPGEE

SEQ ID NO: 86 - protein, 20 aa, synthetic construct
GYTYFTTVTAETLETQPGEE

SEQ ID NO: 87 - protein, 20 aa, synthetic construct
GYTYFTTVTVATLETQPGEE

SEQ ID NO: 88 - protein, 20 aa, synthetic construct
GYTYFTTVTVEALETQPGEE

SEQ ID NO: 89 - protein, 20 aa, synthetic construct
GYTYFTTVTVETAETQPGEE

SEQ ID NO: 90 - protein, 20 aa, synthetic construct
GYTYFTTVTVETLATQPGEE

SEQ ID NO: 91 - protein, 20 aa, synthetic construct
GNTYFTTVTVETLETQPGEE

SEQ ID NO: 92 - protein, 20 aa, synthetic construct
GYNYFTTVTVETLETQPGEE

SEQ ID NO: 93 - protein, 20 aa, synthetic construct
GYTNFTTVTVETLETQPGEE

SEQ ID NO: 94 - protein, 20 aa, synthetic construct
GYTYNTTVTVETLETQPGEE

SEQ ID NO: 95 - protein, 20 aa, synthetic construct
GYTYFNTVTVETLETQPGEE

SEQ ID NO: 96 - protein, 20 aa, synthetic construct
GYTYFTNVTVETLETQPGEE

SEQ ID NO: 97 - protein, 20 aa, synthetic construct
GYTYFTTNTVETLETQPGEE

SEQ ID NO: 98 - protein, 20 aa, synthetic construct
GYTYFTTVNVETLETQPGEE

SEQ ID NO: 99 - protein, 20 aa, synthetic construct
GYTYFTTVTNETLETQPGEE

SEQ ID NO: 100 - protein, 20 aa, synthetic construct
GYTYFTTVTVNTLETQPGEE

SEQ ID NO: 101 - protein, 20 aa, synthetic construct
GYTYFTTVTVENLETQPGEE

SEQ ID NO: 102 - skipped

SEQ ID NO: 103 - protein, 20 aa, synthetic construct
AYTYFTTVTVETLETQPGEE

SEQ ID NO: 104-116 - skipped (13 entries)

SEQ ID NO: 117 - protein, 20 aa, synthetic construct
GYTYFTTVTVETLEAQPGEE

SEQ ID NO: 118 - protein, 20 aa, synthetic construct
GYTYFTTVTVETLETAPGEE

SEQ ID NO: 119 - protein, 20 aa, synthetic construct
GYTYFTTVTVETLETQAGEE

SEQ ID NO: 120 - protein, 20 aa, synthetic construct
GYTYFTTVTVETLETQPAEE

SEQ ID NO: 121 - protein, 20 aa, synthetic construct
GYTYFTTVTVETLETQPGAE

SEQ ID NO: 122 - protein, 20 aa, synthetic construct
GYTYFTTVTVETLETQPGEA

SEQ ID NO: 123 - protein, 20 aa, synthetic construct
GYTYFTTVTVEPYETQPGEE

SEQ ID NO: 124 - protein, 20 aa, synthetic construct
GYTYFTTVTVEMRETQPGEE

SEQ ID NO: 125 - protein, 20 aa, synthetic construct
GYTYFTTVTVEIFETQPGEE

SEQ ID NO: 126 - protein, 20 aa, synthetic construct
GYTYFTTVTVEHFETQPGEE

SEQ ID NO: 127 - protein, 20 aa, synthetic construct
GYTYFTTVTVEWYETQPGEE

SEQ ID NO: 128 - protein, 20 aa, synthetic construct
GYTYFTTVTVEQRETQPGEE

SEQ ID NO: 129 - protein, 20 aa, synthetic construct
GYTYFTTVTVEWFETQPGEE

SEQ ID NO: 130 - protein, 20 aa, synthetic construct
GYTYFTTVTVEERETQPGEE

SEQ ID NO: 131 - protein, 20 aa, synthetic construct
GYTYFTTVTVEDYETQPGEE

SEQ ID NO: 132 - protein, 20 aa, synthetic construct
GYTYFTTVTVEFFETQPGEE

SEQ ID NO: 133 - protein, 20 aa, synthetic construct
GYTYFTTVTVEHYETQPGEE

SEQ ID NO: 134 - protein, 20 aa, synthetic construct
GYTYFTTVTVEMMETQPGEE

SEQ ID NO: 135 - protein, 20 aa, synthetic construct
GYTYFTTVTVEDFETQPGEE

SEQ ID NO: 136 - protein, 20 aa, synthetic construct
GYTYFTTVTVEDIETQPGEE

SEQ ID NO: 137 - protein, 20 aa, synthetic construct
GYTYFTTVTVELIETQPGEE

SEQ ID NO: 138 - protein, 20 aa, synthetic construct
GYTYFTTVTVEEIETQPGEE

SEQ ID NO: 139 - protein, 20 aa, synthetic construct
GYTYFTTVTVEAFETQPGEE

SEQ ID NO: 140 - protein, 20 aa, synthetic construct
GYTYFTTVTVEHHETQPGEE

SEQ ID NO: 141 - protein, 20 aa, synthetic construct
GYTYFTTVTVEPFETQPGEE

SEQ ID NO: 142 - protein, 20 aa, synthetic construct
GYTYFTTVTVEDWETQPGEE

SEQ ID NO: 143 - protein, 21 aa, synthetic construct
GYTYFTTVTVETLETQPGEEA

SEQ ID NO: 144 - protein, 21 aa, synthetic construct
GYTYFTTVTVEPYETQPGEEA

SEQ ID NO: 145 - protein, 21 aa, synthetic construct
GYTYFTTVTVEHFETQPGEEA

SEQ ID NO: 146 - protein, 21 aa, synthetic construct
GYTYFTTVTVEQRETQPGEEA

SEQ ID NO: 147 - protein, 21 aa, synthetic construct
GYTYFTTVTVEWYETQPGEEA

SEQ ID NO: 148 - protein, 21 aa, synthetic construct
GYTYFTTVTVEMRETQPGEEA

SEQ ID NO: 149 - protein, 21 aa, synthetic construct
GYTYFTTVTVEIFETQPGEEA

SEQ ID NO: 150 - skipped

SEQ ID NO: 151 - protein, 9 aa, synthetic construct
LDEVMKTTK

SEQ ID NO: 152 - protein, 9 aa, synthetic construct
IDEVMKTTK

SEQ ID NO: 153 - protein, 21 aa, synthetic construct
NYTYFTTVTVETLETQPGEEA

SEQ ID NO: 154 - protein, 39 aa, synthetic construct
NYTYFTTVTVETLETQPGEEALRKLRKRLLLRKLRKRLL

SEQ ID NO: 155 - protein, 31 aa, synthetic construct
NYTYFTTVTVETLETQPGEEALDEVMKTTKA

SEQ ID NO: 156 - protein, 31 aa, synthetic construct
NYTYFTTVTVEDYETQPGEEALDEVMKTTKA